(19) United States
(12) Patent Application Publication
(10) Pub. No.: US 2023/0256084 A1
(43) Pub. Date: Aug. 17, 2023

(54) SARS-COV-2 IMMUNOGENIC COMPOSITIONS, VACCINES, AND METHODS

(71) Applicants: INSTITUT PASTEUR, PARIS (FR); THERAVECTYS, PARIS (FR)

(72) Inventors: Pierre CHARNEAU, Paris (FR); Min-Wen KU, Paris (FR); Pierre AUTHIE, Paris (FR); Nicolas ESCRIOU, Paris (FR); Maryline BOURGINE, Paris (FR); Laleh MAJLESSI, Paris (FR)

(21) Appl. No.: 18/005,146

(22) PCT Filed: Jul. 15, 2021
(86) PCT No.: PCT/EP2021/069890

§ 371 Date: Jan. 11, 2023

Related U.S. Application Data
(60) Provisional application No. 63/052,264, filed on Jul. 15, 2020.
(60) Provisional application No. 63/130,202, filed on Dec. 23, 2020.

(30) Foreign Application Priority Data
Feb. 2, 2021 (WO) PCT/IB2021/000293

(51) Int. Cl. A61K 39/215; A61P 31/14

(52) U.S. Cl. CPC A61K 39/215; A61P 31/14; A61K 2039/544

(57) ABSTRACT

A method of inducing a protective immune response against Severe Acute Respiratory Syndrome beta-coronavirus 2 (SARS-CoV-2), comprising administering to the upper respiratory tract of a subject an effective amount of an agent that induces a protective immune response against SARS-CoV-2. A dosage form for administration to the upper respiratory tract of a pseudotyped lentiviral vector particle encoding a Severe Acute Respiratory Syndrome beta-coronavirus 2 (SARS-CoV-2) spike (S) protein or a derivative or fragment thereof.

### BACKGROUND

The new Severe Acute Respiratory Syndrome beta-coronavirus 2 (SARS-CoV-2), emerged in late 2019 in Wuhan, China, is extraordinarily contagious and fast-spreading across the world (Guo et al., 2020). Compared to the previously emerged SARS or Middle East Respiratory Syndrome (MERS) coronaviruses, SARS-CoV-2 causes unprecedented threat on global health and tremendous socio-economic consequences. Therefore, the development of effective prophylactic vaccines against SARS-CoV-2 is of absolute imperative to contain the spread of the epidemic and to attenuate the onset of CoronaVirus Disease 2019 (COVID-19), such as deleterious inflammation and progressive respiratory failure (Amanat and Krammer, 2020). Although lung is the organ of predilection for SARS-CoV-2, its neurotropism, like that of SARS-CoV and Middle East Respiratory Syndrome (MERS)-CoV, (Glass et al., 2004; Li et al., 2016; Netland et al., 2008) has been reported (Aghagoli et al., 2020; Fotuhi et al., 2020; Hu et al., 2020; Liu et al., 2020; Politi et al., 2020; Roman et al., 2020; von Weyhern et al., 2020; Whittaker et al., 2020). Moreover, expression of Angiotensin Converting Enzyme 2 (ACE2) in neuronal and glial cells has been described (Chen et al., 2020; Xu and Lazartigues, 2020). Accordingly, COVID-19 human patients can present symptoms like headache, myalgia, anosmia, dysgeusia, impaired consciousness and acute cerebrovascular disease (Bourgonje et al., 2020; Hu et al., 2020; Mao et al., 2020). Viruses can gain access to the brain through neural dissemination or hematogenous route (Desforges et al., 2014). Analysis of autopsies of COVID-19 deceased patients demonstrated presence of SARS-CoV-2 in nasopharynx and brain and virus entry into central nervous system (CNS) via neural-mucosal interface of olfactory mucosa (Meinhardt et al., 2020). Therefore, it is critical to focus hereinafter on the protective properties of COVID-19 vaccine candidates, not only in the respiratory tracts, but also in the brain.

Coronaviruses are enveloped, non-segmented positive-stranded RNA viruses, characterized by their envelop-anchored Spike (S) glycoprotein (Walls et al., 2020). The SARS-CoV-2 S (S_{CoV-2}) is a (180 kDa)₃ homotrimeric class I viral fusion protein, which engages the carboxypeptidase Angiotensin-Converting Enzyme 2 (ACE2), expressed on host cells. The monomer of S_{CoV-2} protein possesses an ecto-domain, a transmembrane anchor domain, and a short internal tail. S_{CoV-2} is activated by a two-step sequential proteolytic cleavage to initiate fusion with the host cell membrane. Subsequent to S_{CoV-2}-ACE2 interaction, which leads to a conformational reorganization, the extracellular domain of S_{CoV-2} is first cleaved at the highly specific furin 682^{RRAR}685 (SEQ ID NO: 99) site (Guo et al., 2020; Walls et al., 2020), a key factor determining the pathological features of the virus, linked to the ubiquitous furin expression (Wang et al., 2020). The resulted subunits are constituted of: (i) S1, which harbors the ACE2 Receptor Binding Domain (RBD), with the atomic contacts restricted to the ACE2 protease domain and also harbors main B-cell epitopes, targeted of NAbs (Walls et al., 2020), and (ii) S2, which bears the membrane-fusion elements. Like for S_{CoV-1}, the shedding of S1 renders accessible on S2 the second proteolytic cleavage site 7978, namely S2′ (Belouzard et al., 2009). According to the cell or tissue types, one or several host proteases, including furin, trypsin, cathepsins or TransMembrane Protease Serine Protease (TMPRSS)-2 or -4, can be involved in this second cleavage step (Coutard et al., 2020). The consequent “fusogenic” conformational changes of S result in a highly stable postfusion form of S_{CoV-2} that initiates the fusion reaction with the host cell membrane (Sternberg and Naujokat, 2020) and lead to the exposure of a Fusion Peptide (FP), adjacent to S2′. Insertion of FP to the host cell/vesicle membrane primes the fusion reaction, whereby the viral RNA release into the host cytosol (Lai et al., 2017). The facts that the S_{CoV-2}-ACE2 interaction is the only mechanism, thus far identified for the host cell infection by SARS-CoV-2, and that the RBD contains numerous conformational B-cell epitopes (Walls et al., 2020), designate this viral envelop glycoprotein as the main target for neutralization antibodies (nAbs). Like envelop glycoproteins of several other viruses including respiratory syncytial virus, HIV, Ebola virus, human metapneumovirus, and Lassa virus (Bos et al., 2020), it is possible to engineer S_{CoV-2} to avoid its conformational dynamics and its stabilization under its prefusion conformation that will possibly better maintain exposure of the S1 B-cell epitopes and possibly improve immunogen availability (McCallum et al., 2020).

Several vaccine alternatives have significant drawbacks. Specifically: (i) attenuated or inactivated viral vaccine candidates which require extensive safety testing, (ii) the nucleic acids encoding for S do not have proven efficacy on long term protection, (iii) protein vaccines require the use of adjuvants and boosting, and (iv) pre-existing immunity exists for viral vectors, such as adenoviral vectors, can generate strong anti-vector immune response, which largely reduces their immunogenicity (Rosenberg et al., 1998; Schirmbeck et al., 2008).

Among viral vectors, lentiviral vectors exist under integrative (ILV) and non-integrative (NILV) forms which are permissive to insertion of up to 8 kb-length transgenes of vaccinal interest and possess outstanding potential of gene transfer to the nuclei of host cells (Di Nunzio et al., 2012; Hu et al., 2011; Ku et al., 2020; Zennou et al., 2000). Lentivectors display in vivo tropism for immune cells, notably dendritic cells, are non-replicative, non-cytopathic and scarcely inflammatory, and induce long-lasting B- and T-cell immunity (Di Nunzio et al., 2012; Hu et al., 2011; Ku et al., 2020; Zennou et al., 2000). Pseudo-typed at their envelop with the surface glycoprotein of Vesicular Stomatitis Virus, to which the human population has been barely exposed, LV are not target of specific preexisting immunity in humans, in net contrast to adenoviral vectors (Rosenberg et al., 1998; Schirmbeck et al., 2008). In addition, the safety of LV has been established in human in a phase I/II Human Immunodeficiency Virus (HIV)-1 vaccine trial (2011-006260-52 EN).

A need exists for compositions and methods of inducing a protective immune response against SARS-CoV-2. This disclosure meets these and other needs.

### SUMMARY

To develop a vaccine candidate capable of preventing COVID-19 or decreasing its severity, LV coding for: (i) full-length, membrane anchored form of S (LV::S_{FL}/LV::S_{FL}), (ii) S1-S2 ecto-domain, without the transmembrane and internal tail domains (LV::S1-52), (iii) S1 alone (LV::S1), (iv) mutated S deleted of a sequence encompassing the furin site and substituted at residues K⁹⁸⁶P and V⁹⁸⁷P to introduce consecutive proline residues in S2 (2P mutation) (LV::S_{ΔF2P}) thereby providing a stabilized (2P) and prefusion (ΔF) form of the protein were generated. Additional vaccine candidates were generated, including LV coding for: (i) the spike protein of variant B1.351 (so called South African or β variant), (ii) the spike protein of variant B1.1.7 (so called UK or alpha variant), (iii) the spike protein of variant B1.351 substituted at residues K⁹⁸⁶P and V⁹⁸⁷P, (iv) the full-length, membrane anchored form of S combined with a D614G substitution (LV::S_{FL}-D614G), and (v) the spike protein of variant P.1 (so called Manaus or gamma variant). The data presented in the examples establish in particular that LV::S_{FL} and LV::S_{ΔF2P} either in the integrative or non integrative version of the vector(i) induced neutralizing antibodies specific to the Spike glycoprotein (S) of SARS-CoV-2, the etiologic agent of COVID-19, with neutralizing activity comparable to those found in a cohort of SARS-CoV-2 patients, and (ii) induced Spike-specific CD8+ T cells. Moreover, using golden hamsters highly susceptible to SARS-CoV-2 replication, a strong prophylactic effect of LV::S_{FL} or LV::S_{ΔF2P} immunization against the replication of a SARS-CoV-2 clinical isolate was demonstrated. Similar results were obtained in a mouse model in which the expression of human ACE2 (hACE2) was induced in the respiratory tracts by an adenoviral vector serotype 5 (Ad5). Besides, in transgenic mice generated as a preclinical model showing unprecedent permissibility to SARS-CoV-2 replication including in brain, the inventors were able to demonstrate that a LV encoding a prefusion form of spike glycoprotein of SARS-CoV-2 such as LV::S_{ΔF2P} induces substantial protection of respiratory tracts and CNS against SARS-CoV-2. Unexpectedly the generated transgenic mice enabled addressing the capability of protection of the CNS by the developed LV encoding the Spike protein or a derivative or a fragment thereof according to the definition provided below and illustrated in the experimental examples. In addition, the inventors have demonstrated that a single intranasal administration of a LV encoding a prefusion form of Spike glycoprotein of SARS-CoV-2 induces substantial protection of respiratory tracts and totally avoids pulmonary inflammation in the susceptible hamster model. Importantly also, the upper respiratory tract mucosal boost/target immunization with LV::S_{FL} or with LV::S_{ΔF2P} was instrumental in the protection efficacy in stringent preclinical model constituted by the generated transgenic mice. The presented virological, immunological and histopathological data demonstrates: (i) marked prophylactic effects of a LV-based vaccination strategy against SARS-CoV-2, (ii) the fact that LV-based immunization represents a promising strategy to develop vaccine candidates against coronaviruses, and (iii) mucosal immunization enables vigorous protective lung immunity and protective CNS immunity. In the particular context of SARS-CoV-2 exhibiting tropism for multiple organs in the infected host, lentiviral vector in any of its forms harboring the lentiviral sequences essential for targeting host cells and enabling expression of a transgene, for instance encoding the Spike protein of SARS-CoV-2 or a derivative or fragment thereof bearing B epitopes and T epitopes, has shown capability to induce and/or activate immune response against the transgene antigen. The inventors have in particular proven the capability of the lentiviral vector to retain or support a conformation of the S antigen (whether wild type or mutated as disclosed herein) that enables effective presentation of the epitopes, especially of the B-epitopes, to the immune system of the host. In addition, the experimental data disclosed herein show that an administration route encompassing a step of administration to upper respiratory tract of the host may improve the immune response in some tissues or organs targeted by the virus. These results are surprising and unexpected.

The data in the examples also demonstrate: (i) strong CD8⁺ T-cell responses induced by NILV::S_{CoV-2 Wuhan} at the systemic level, (ii) notable proportions of IFN-γ-producing lung CD8⁺ T cells, specific to several S_{CoV-2} epitopes, (iii) high proportions of lung CD8⁺ T cells with effector memory (Tem) and resident memory (Trm) phenotye, (iv) recruitment of CD8⁺ T cells in the olfactory bulbs, detectable in mice vaccinated and challenged with SARS-CoV-2 Wuhan or SARS-CoV-2 P.1 variant. Remarkably, all murine and human CD8⁺ T-cell epitopes identified on S_{CoV-2 Wuhan} sequence are preserved in the mutated S_{CoV-2 Manaus P.1}. These observations indicate the strong potential of NILV at inducing full protection of lungs and brain against ancestral and emerging SARS-CoV-2 variants by eliciting marked B and T cell-responses. In contrast to the B-cell epitopes which are targets of NAbs, the so far identified T-cell epitopes have not been impacted by mutations accumulated in the S_{CoV-2} of the emerging variants. These results are surprising and unexpected.

The data in the examples further demonstrate: (i) sera from mice immunized with LV::S_{CoV-2 B1.1.7} neutralized at high EC₅₀ pseudo-viruses harboring S_{CoV-2 Wuhan} and LV::S S_{CoV-2 B1.1.7}, but poorly pseudo-viruses harboring S_{CoV-2 B1.351} and LV::S S_{CoV-2 P.1}.

(ii) sera from mice immunized with LV::S S_{CoV-2 P.1} neutralized at high EC₅₀ pseudo-viruses harboring S_{CoV-2 P.1} and LV::S_{CoV-2 B1.351}, but poorly pseudo-viruses harboring S_{CoV-2 Wuhan} and LV::S_{CoV-2 B1.1.7}.

(iii) sera from mice immunized with LV::S_{CoV-2 B1.351} not only neutralized at high EC₅₀ pseudo-viruses carrying S_{CoV-2 P.1} and LV::S_{CoV-2 B1.351} but also pseudo-viruses harboring S_{CoV-2 Wuhan} and LV::S_{CoV-2 B1.1.7}.

These results designate the Spike sequence from the B1.351 (South African or β) variant as the most cross-reactive immunogen in terms of neutralizing antibodies.

Furthermore, the data showed that in the context of LV, Spike stabilization by K986P-V987P substitutions (2P) considerably improves the (cross) neutralizing antibody activity.

Taken together the data surprisingly and unexpectedly show that one particularly effective antigen is the full-length Spike from the B1.351 (South African or β) variant with 2P.

Accordingly, in a first aspect this invention provides a method of inducing a protective immune response against Severe Acute Respiratory Syndrome beta-coronavirus 2 (SARS-CoV-2) in a subject, comprising administering to the upper respiratory tract of the subject an effective amount of an agent that induces a protective immune response against SARS-CoV-2. In certain embodiments the agent that induces a protective immune response against SARS-CoV-2 is a pseudotyped LV vector particle encoding a SARS-CoV-2 Spike (S) glycoprotein or a derivative or fragment thereof. In some embodiments the agent is administered by aerosol inhalation. In some embodiments the agent is administered by nasal instillation. In some embodiments the agent is administered by nasal insufflation. In some embodiments the treatment course consists of a single administration to the upper respiratory tract. In some embodiments the treatment course comprises at least one priming administration outside the respiratory tract followed by at least one boosting administration to the upper respiratory tract. In some embodiments the protective immune response comprises production of SARS-CoV-2 neutralizing antibodies in the subject. In some embodiments the neutralizing antibodies comprise IgG antibodies. In some embodiments the neutralizing antibodies comprise IgA antibodies. In some embodiments the protective immune response comprises production of SARS-CoV-2 S-specific T cells in the subject. In some embodiments the SARS-CoV-2 S-specific T cells comprise CD4⁺ T cells, CD8⁺ T cells, or both CD4⁺ and CD8⁺ T cells. In some embodiments the SARS-CoV-2 S-specific T cells comprise lung CD8+ T cells. In some embodiments the SARS-CoV-2 S-specific T cells comprise IFN-γ-producing T-cells. In some embodiments the CD8+ T cells comprise T cells with an effector memory (Tem) and/or resident memory (Trm) phenotype. In some embodiments the SARS-CoV-2 S-specific T cells are recruited to the olfactory bulb. In some embodiments the protective immune response provides a reduced likelihood of developing SARS-CoV-2 infection-related inflammation in the subject. In some embodiments the SARS-CoV-2 S protein derivative has an amino acid sequence at least 95% identical to SEQ ID NO: 1. In some embodiments the SARS-CoV-2 S protein derivative is expressed from a coding sequence having a nucleotide sequence at least 80% identical to SEQ ID NO: 2. In some embodiments the SARS-CoV-2 S protein fragment comprises a peptide selected from peptide 61-75 (NVTWFHAIHVSGTNG (SEQ ID NO: 15)), peptide 536-550 (NKCVNFNFNGLTGTG (SEQ ID NO: 16)) and peptide 576-590 (VRDPQTLEILDITPC (SEQ ID NO: 17)). In some embodiments the SARS-CoV-2 S derivative or fragment thereof comprises an amino acid modification relative to SEQ ID NO: 1, the modification selected from: (i) 986^{K→P} and 987^{V→P}, (ii) 681^{PRRARS}686 (SEQ ID NO: 22)→681^{PGSAGS}686 (SEQ ID NO: 23), and (iii) 986^{K→}P, 987V^{→P}, and 675^{QTQTNSPRRAR}685 (SEQ ID NO: 24) deletion. In some embodiments, the Severe Acute Respiratory Syndrome beta-coronavirus 2 (SARS-CoV-2) spike (S) protein or a derivative or fragment thereof comprises or consists of an amino acid sequence selected from SEQ ID NOS: 1, 5, 8, 11, 14, 108, 111, 114, 117, and 120.

In some embodiments the SARS-CoV-2 S protein or a derivative or fragment thereof comprises of SEQ ID NO: 1. In some embodiments the SARS-CoV-2 S protein or a derivative or fragment thereof consists of SEQ ID NO: 1.

In some embodiments the SARS-CoV-2 S protein or a derivative or fragment thereof comprises of SEQ ID NO: 5. In some embodiments the SARS-CoV-2 S protein or a derivative or fragment thereof consists of SEQ ID NO: 5.

In some embodiments the SARS-CoV-2 S protein or a derivative or fragment thereof comprises of SEQ ID NO: 8. In some embodiments the SARS-CoV-2 S protein or a derivative or fragment thereof consists of SEQ ID NO: 8.

In some embodiments the SARS-CoV-2 S protein or a derivative or fragment thereof comprises of SEQ ID NO: 11. In some embodiments the SARS-CoV-2 S protein or a derivative or fragment thereof consists of SEQ ID NO: 11.

In some embodiments the SARS-CoV-2 S protein or a derivative or fragment thereof comprises of SEQ ID NO: 14. In some embodiments the SARS-CoV-2 S protein or a derivative or fragment thereof consists of SEQ ID NO: 14.

In some embodiments the SARS-CoV-2 S protein or a derivative or fragment thereof comprises of SEQ ID NO: 108. In some embodiments the SARS-CoV-2 S protein or a derivative or fragment thereof consists of SEQ ID NO: 108.

In some embodiments the SARS-CoV-2 S protein or a derivative or fragment thereof comprises of SEQ ID NO: 111. In some embodiments the SARS-CoV-2 S protein or a derivative or fragment thereof consists of SEQ ID NO: 111.

In some embodiments the SARS-CoV-2 S protein or a derivative or fragment thereof comprises of SEQ ID NO: 114. In some embodiments the SARS-CoV-2 S protein or a derivative or fragment thereof consists of SEQ ID NO: 114.

In some embodiments the SARS-CoV-2 S protein or a derivative or fragment thereof comprises of SEQ ID NO: 117. In some embodiments the SARS-CoV-2 S protein or a derivative or fragment thereof consists of SEQ ID NO: 117.

In some embodiments the SARS-CoV-2 S protein or a derivative or fragment thereof comprises of SEQ ID NO: 120. In some embodiments the SARS-CoV-2 S protein or a derivative or fragment thereof consists of SEQ ID NO: 120.

In some embodiments the administered LV vector particle is integrative (ILV). In some embodiments the administered lentiviral vector particle is nonintegrative with a defective integrase protein (NILV). In some embodiments the administered NILV comprises a D64V mutation in an integrase coding sequence. In some embodiments the administered LV vector particle is pseudotyped with Vesicular Stomatitis Virus envelop Glycoprotein (VSV-G). In some embodiments the LV vector particle is administered as a vaccine formulation comprising the LV vector particle and a pharmaceutically acceptable carrier.

In another aspect, the invention relates to a dosage form for administration to the upper respiratory tract of a subject of a pseudotyped LV vector particle encoding a SARS-CoV-2 Spike (S) protein or a derivative or fragment thereof. In some embodiments the dosage form is for administration by aerosol inhalation. In some embodiments the dosage form is for administration by nasal instillation. In some embodiments the dosage form is for administration by nasal insufflation. In some embodiments the SARS-CoV-2 S protein derivative has an amino acid sequence at least 95% identical to SEQ ID NO: 1. In some embodiments the SARS-CoV-2 S protein derivative is expressed from a coding sequence having a nucleotide sequence at least 80% identical to SEQ ID NO: 2. In some embodiments the SARS-CoV-2 S protein fragment comprises a peptide selected from peptide 61-75 (NVTWFHAIHVSGTNG (SEQ ID NO: 15)), peptide 536-550 (NKCVNFNFNGLTGTG (SEQ ID NO: 16)) and peptide 576-590 (VRDPQTLEILDITPC (SEQ ID NO: 17)). In some embodiments the SARS-CoV-2 S derivative or fragment thereof comprises an amino acid modification relative to SEQ ID NO: 1, the modification selected from: (i) 986^{K→P} and 987^{V→P}, (ii) 681^{PRRARS}686 (SEQ ID NO: 22)→681^{PGSAGS}686 (SEQ ID NO: 23), and (iii) 986^{K→}P, 987V^{→P}, and 675^{QTQTNSPRRAR}685 (SEQ ID NO: 24) deletion. Additional derivatives and fragments of the S protein are disclosed below along with various aspects of the invention.

In some embodiments the administered LV vector particle is integrative (ILV). In some embodiments the administered LV vector particle is nonintegrative (NILV). In some embodiments the NILV particle comprises a D64V mutation in an integrase coding sequence. In some embodiments the lentiviral vector particle is pseudotyped with Vesicular Stomatitis Virus envelop Glycoprotein (VSV-G).

In another aspect, a kit is provided. The kit may be suitable for use in practicing a method disclosed herein. In some embodiments the kit comprises a dosage form for administration to the upper respiratory tract of a subject of the pseudotyped LV vector particle encoding a SARS-CoV-2 S protein or a derivative or fragment thereof according to this disclosure. In some embodiments the applicator for administration is an applicator for aerosol inhalation. In some embodiments the applicator for administration to the upper respiratory tract of a subject is an applicator for nasal instillation. In some embodiments the applicator for administration to the upper respiratory tract of a subject is an applicator for nasal insufflation.

Also provided are novel and nonobvious pseudotyped LV vector particles encoding a SARS-CoV-2 Spike (S) protein or a derivative or fragment thereof. In some embodiments the pseudotyped LV vector particles are administered to the upper respiratory tract of a subject. In some embodiments the pseudotyped LV vector particles induce a protective immune response providing a reduced likelihood of developing SARS-CoV-2 infection-related inflammation following administration to the upper respiratory tract of a subject. In some embodiments the SARS-CoV-2 S protein derivative has an amino acid sequence at least 95% identical to SEQ ID NO: 1. In some embodiments the SARS-CoV-2 S protein derivative is expressed from a coding sequence having a nucleotide sequence at least 80% identical to SEQ ID NO: 2. In some embodiments the SARS-CoV-2 S protein fragment comprises a peptide selected from Peptide 61-75 (NVTWFHAIHVSGTNG—SEQ ID No.15), peptide 536-550 (NKCVNFNFNGLTGTG—SEQ ID No.16) and peptide 576-590 (VRDPQTLEILDITPC—SEQ ID No.17). In some embodiments the SARS-CoV-2 S derivative or fragment thereof comprises an amino acid modification relative to SEQ ID NO: 1, the modification selected from: (i) 986^{K→P} and 987^{V→P}, (ii) 681^{PRRARS}686 (SEQ ID NO: 22)→681^{PGSAGS}686 (SEQ ID NO: 23), and (iii) 986^{K→}P, 987V^{→P}, and 675^{QTQTNSPRRAR}685 (SEQ ID NO: 24) deletion. In some embodiments the LV vector particle is integrative (ILV). In some embodiments the lentiviral vector particle is nonintegrative (NILV). In some embodiments the NILV particle comprises a D64V mutation in an integrase coding sequence. In some embodiments the LV vector particle is pseudotyped with Vesicular Stomatitis Virus envelop Glycoprotein (VSV-G). In some embodiments, the pseudotyped LV vector particle encodes a Spike glycoprotein, or fragment or derivative thereof, that has the same amino acid sequence as the spike protein, or fragment or derivative thereof, that is encoded by vector selected from:

pFlap-ieCMV-S2PAF-WPREm (also named pFlap-ieCMV-S2PdeltaF-WPREm) (CNCM I-5537), pFlap-ieCMV-S2P3F-WPREm (CNCM I-5538), pFlap-ieCMV-S2P-WPREm (CNCM I-5539), pFlap-ieCMV-SFL-WPREm (CNCM I-5540), pFlap-ieCMV-S-B1.1.7-WPREm (CNCM I-5708), pFlap-ieCMV-S-B351-WPREm (CNCM I-5709), pFlap-ieCMV-S-B351-2P-WPREm (CNCM I-5710), pFlap-ieCMV-SFL-D614G-WPREm (CNCM I-5711), and pFlap-ieCMV-S-P1-WPREm (CNCM I-5712).

Also provided is a vector selected from: pFlap-ieCMV-S2PAF-WPREm (also named pFlap-ieCMV-S2PdeltaF-WPREm) (CNCM I-5537), pFlap-ieCMV-S2P3F-WPREm (CNCM I-5538), pFlap-ieCMV-S2P-WPREm (CNCM I-5539), pFlap-ieCMV-SFL-WPREm (CNCM I-5540), pFlap-ieCMV-S-B1.1.7-WPREm (CNCM I-5708), pFlap-ieCMV-S-B351-WPREm (CNCM I-5709), pFlap-ieCMV-S-B351-2P-WPREm (CNCM I-5710), pFlap-ieCMV-SFL-D614G-WPREm (CNCM I-5711), and pFlap-ieCMV-S-P1-WPREm (CNCM I-5712).

Also provided is a host cell comprising a vector selected from: pFlap-ieCMV-S2PΔF-WPREm (CNCM I-5537), pFlap-ieCMV-S2P3F-WPREm (CNCM I-5538), pFlap-ieCMV-S2P-WPREm (CNCM I-5539), pFlap-ieCMV-SFL-WPREm (CNCM I-5540), pFlap-ieCMV-S-B1.1.7-WPREm (CNCM I-5708), pFlap-ieCMV-S-B351-WPREm (CNCM I-5709), pFlap-ieCMV-S-B351-2P-WPREm (CNCM I-5710), pFlap-ieCMV-SFL-D614G-WPREm (CNCM I-5711), pFlap-ieCMV-S-P1-WPREm (CNCM I-5712). In some embodiments the vector is stably integrated into the host cell genome, while in other embodiments it is not.

Also provided is a pseudotyped LV vector particle encoding a SARS-CoV-2 Spike (S) glycoprotein or a derivative or fragment thereof, wherein the pseudotyped LV vector particle is made by a method comprising co-transfection of a host cell with a vector selected from: pFlap-ieCMV-S2PΔF-WPREm (CNCM I-5537), pFlap-ieCMV-S2P3F-WPREm (CNCM I-5538), pFlap-ieCMV-S2P-WPREm (CNCM I-5539), pFlap-ieCMV-SFL-WPREm (CNCM I-5540), pFlap-ieCMV-S-B1.1.7-WPREm (CNCM I-5708), pFlap-ieCMV-S-B351-WPREm (CNCM I-5709), pFlap-ieCMV-S-B351-2P-WPREm (CNCM I-5710), pFlap-ieCMV-SFL-D614G-WPREm (CNCM I-5711), and pFlap-ieCMV-S-P1-WPREm (CNCM I-5712).

### BRIEF DESCRIPTION OF THE DRAWINGS—The figures are filed as color figures

FIG. 1. Induction of anti-S_{CoV-2} Ab responses by LV. (A) Schematic representation of 3 forms of S_{CoV-2} protein (S_{FL}, S1-S2 and S1) encoded by LV injected to mice. RBD, S1/S2 and S2′ cleavage sites, Fusion Peptide (FP), TransMembrane (TM) and short internal tail (T) are indicated. (B) Dynamic of anti-S_{CoV-2} Ab response following LV immunization. C57BL/6 mice (n=4/group) were injected i.p. with 1×10⁷ TU of LV::GFP as a negative control, LV::S1, LV::S1-S2, or LV::S_{FL}. Sera were collected at 2, 3, 4 and 6 weeks post immunization. Anti-S_{CoV-2} IgG responses were evaluated by ELISA and expressed as mean endpoint dilution titers. (C) Neutralization capacity of anti-S_{CoV-2} Abs induced by LV::S_{FL} immunization. Mouse sera were evaluated in a sero-neutralization assay to determine 50% effective concentration (EC50) neutralizing titers. (D) Correlation between the Ab titers and neutralization activity in various experimental groups. Statistical significance was determined by two-sided Spearman rank-correlation test. NS: not significant. (E) Head-to-head comparison at a 1:40 dilution between mouse sera taken at weeks 3 or 4 after immunization and a cohort of mildly symptomatic individuals living in Crépy-en-Valois, Ile de France. These patients did not seek medical attention and recovered from COVID-19. Results are expressed as mean±SEM percentages of inhibition of luciferase activity.

FIG. 2. Induction of T-cell responses by LV::S_{FL}. C57BL/6 mice (n=3) were immunized i.p. with 1×10⁷ TU of LV::S_{FL} or a negative control LV. (A) Splenocytes collected 2 weeks after immunization were subjected to an IFN-γ ELISPOT using 16 distinct pools of 15-mer peptides spanning the entire S_{CoV-2} (1-1273 a.a.) and overlapping each other by 10 a.a. residues. SFU=Spot-Forming Cells. (B) Deconvolution of the 16 positive peptide pools by ELISPOT applied to splenocytes pooled from 3 LV::S_{FL}- or Ctrl LV-immunized mice. (C) Intracellular IFN-γ versus IL-2 staining of CD4⁺ or CD8⁺ T splenocytes after stimulation with individual peptides encompassing the immunodominant epitopes.

FIG. 3. Set up of a murine model expressing hACE2 in the respiratory tracts. (A) Detection of hACE2 expression by RT-PCR in HEK293 T cells transduced with Ad5::hACE2, at 2 days post transduction. NT: Not transduced. (B) hACE2 protein detection by Western Blot in lung cell extracts recovered at day 4 after i.n. instillation of Ad5::hACE2 or empty Ad5 to C57BL/6 mice (n=2/group). (C) GFP expression in lung cells prepared at day 4 after i.n. instillation of Ad5::GFP or PBS into C57BL/6 mice, as assessed by flow cytometry in the CD45⁺ hematopoietic or EpCam⁺ epithelial cells. (D) Lung viral loads in mice pretreated with 2.5×10⁹ IGU of Ad5::hACE2, control empty Ad5 or PBS followed by i.n. inoculation of 1×10⁵ TCID₅₀ of SARS-CoV-2 4 days later. In one group, the Ad5::hACE2-pretreated mice were inoculated with an equivalent amounts of heat-killed (HK) virus to measure the input viral RNA in the absence of viral replication. Viral load quantitation by qRT-PCR in the lung homogenates at 2, 4 or 7 dpi. The red line indicates the detection limit. (E) Percentages of CD45⁺ cells in the lungs, as determined 4 days after pretreatment with various doses of Ad5::hACE2. (F) Lung viral loads in mice pretreated with various doses of Ad5::hACE2, followed by i.n. inoculation of 1×10⁵ TCID₅₀ of SARS-CoV-2 4 days later. Viral load were determined at 3 dpi.

FIG. 4. Protective potential of systemic immunization with LV::S_{FL} against SARS-CoV-2 in mice. (A) Timeline of vaccination by a single i.p. injection of LV followed by Ad5::hACE2 pretreatment and i.n. SARS-CoV-2 challenge. (B) Lung viral loads in unvaccinated mice (PBS), LV::S_{FL}- or sham-vaccinated mice, at 3 dpi. Statistical significance of the differences in the viral loads was evaluated by two tailed unpaired t test; *=p<0.0139.

FIG. 5. Intranasal boost with LV::S_{FL} strongly protects against SARS-CoV-2 in mice. (A) Timeline of the prime-boost strategy based on LV, followed by Ad5::hACE2 pretreatment and SARS-CoV-2 challenge. (B) Titers of anti-S_{CoV-2} IgG, as quantitated by ELISA in the sera of C57BL/6 mice primed i.p. at week 0 and boosted i.p. or i.n. at week 3 (left). Titers were determined as mean endpoint dilution before boost (week 3) and challenge (week 4). *** p<0.001, **** p<0.0001; two-way ANOVA followed by Sidak's multiple comparison test. NS, not significant. Neutralization capacity of these sera, indicated as EC50 (right). (C). Lung viral loads at 3 dpi in mice primed (i.p.) and boosted (i.p. or i.n.) with LV::S_{FL}. Sham-vaccinated received an empty LV. The red line indicates the detection limit. Statistical significance of the differences in the viral loads was evaluated by two tailed unpaired t test; *=p<0.0139, ***=p<0.0088. (D) Titers of anti-S_{CoV-2} IgG and IgA Abs determined in the clarified lung homogenates by ELISA, by use of a foldon-trimerized S_{CoV-2} for coating. (E) Neutralizing activity of the clarified lung homogenates, determined for ⅕ dilution. Statistical significance of the difference was evaluated by Mann-Whitney U test (*=p<0.0159).

FIG. 6. LV::S_{FL} vaccination reduces SARS-Co-2-mediated lung inflammation in mice. (A) Flow cytometric strategy to identify and quantify distinct lung innate immune cell subsets. Lung hematopoietic CD45⁺ cells were analyzed by use of antibodies specific to surface markers, or combination of surface markers, allowing characterization of innate immune cell populations, via 3 distinct paths and by sequential gating. The cell populations are highlighted in grey. (B) Percentages of each innate immune subset versus total lung CD45⁺ cells at 3 dpi in mice sham-vaccinated or vaccinated with LV::S_{FL}, following various prime-boost regimen compared to non-infected (NI) controls which only received PBS. All mice were pretreated with Ad5::hACE-2, 4 days prior to SARS-CoV-2 inoculation. (C) Relative log 2 fold change in cytokines and chemokines mRNA expression in mice sham-vaccinated or vaccinated with LV::S_{FL}, following various prime-boost regimen at 3 dpi. Data were normalized versus PBS-treated, unchallenged controls. Statistical significance was evaluated by two tailed unpaired t test; *=p<0.05, **=p<0.01, ***=p<0.001 and ****=p<0.0001.

FIG. 7. Intranasal vaccination with LV::S_{FL} strongly protects against SARS-CoV-2 in golden hamsters. (A) Timeline of the LV::S_{FL} prime-boost/target immunization regimen and SARS-CoV-2 challenge in hamsters. Sham-vaccinated received an empty LV. (B) Dynamic of anti-S_{CoV-2} Ab response following LV immunization. Sera were collected from sham- or LV-vaccinated hamsters at 3, 5 (pre-boost), and 6 (post-boost) weeks after the prime injection. Anti-S_{CoV-2} IgG responses were evaluated by ELISA and expressed as mean endpoint dilution titers. (C) Post boost/target EC50 neutralizing titers, determined in the hamsters' sera after boost, and as compared to the sera from a cohort of asymptomatic (AS), pauci-symptomatic (PS), symptomatic COVID-19 cases (S) or hospitalized (H) humans. (D) Weight follow-up in hamsters, either sham- or LV::S_{FL}-vaccinated with diverse regimens. For further clarity, only the individuals reaching 4 dpi are shown. Those sacrificed at 2 dpi had the same mean weight as their counterparts of the same groups between 0 and 2 dpi. (E) Lung viral loads at 2 or 4 dpi with SARS-CoV-2 in LV::S_{FL}-vaccinated hamsters. Statistical significance of the differences in the viral loads was evaluated by two tailed unpaired t test; *=p<0.0402, ****=p<0.0001. See also Figure S4C. (F) Relative log₂ fold changes in cytokines and chemokines expression in LV::S_{FL}-vaccinated and protected hamsters versus unprotected sham-vaccinated individuals, as determined at 4 dpi by qRT-PCR in the total lung homogenates and normalized versus untreated controls. Statistical significance of the differences in cytokines and chemokines level was evaluated by one-way ANOVA; *=p<0.05, **=p<0.01.

FIG. 8. LV::S_{FL} vaccination reduces SARS-Co-2-mediated histopathology in golden hamsters. Animals are those detailed in the FIG. 6. (A) Determination of log 2 fold change in cytokines and chemokines mRNA expression in mice sham-vaccinated or vaccinated with LV::S_{FL}, following various prime-boost regimen. The same order of appearance for each construct and regimen applies in each determination. (B) Histological analysis HE&S lung shown for 2 and 4 dpi. Original magnification: ×10, scale bar: 100 μm. Br: Bronchi or bronchiole. By: Blood vessel. Arrow: Mononuclear inflammatory cell infiltration. Star: Degenerative changes in the respiratory epithelium. (C) Heatmap recapitulating the average of histological scores, for each defined parameter and determined for individuals of the same groups at 2 or 4 dpi.

FIG. 9. Protective efficacy of NILV::S_{FL} in a systemic prime and intranasal boost regimen in golden hamsters. (A) Timeline of the NILV::S_{FL} prime-boost/target immunization regimen and SARS-CoV-2 challenge in hamsters. (B) Profile of serum anti-S_{CoV-2} IgG response following a single (i.m.) injection or a prime (i.m.)-boost (i.n.) immunization with NILV::S_{FL}. Anti-S_{CoV-2} IgG responses were expressed as mean endpoint dilution titers. (C) Lung viral loads at 4 dpi with SARS-CoV-2 in controls or NILV::S_{FL}-vaccinated hamsters. Statistical significance of the differences in the viral loads was evaluated by two tailed unpaired t test; **=p<0.01. (D) Post boost/target EC50 neutralizing titers, determined in the hamsters' sera. (E) Lung histological analysis was performed by H&E. Heatmap recapitulating the histological scores, for each parameter and determined for individuals of various groups at 4 dpi. (F, G) Representative whole-lung section from NILV::S_{FL} i.m.-NILV::S_{FL} i.n. (F) or sham i.m.-sham i.n. (G) hamsters at 4 dpi.

FIG. 10. Maps of plasmids used for production of LV encoding S_{FL}, S1-S2 or S1 antigens.

FIG. 11. Schematic representation of S_{FL} and S_{ΔF2P} encoded by LV. RBD, S1/S2 and S2′ cleavage sites, Fusion Peptide (FP), TransMembrane domain (TM) and short internal tail (T), 675^{QTQTNSPRRAR}685 (SEQ ID NO: 24) sequence encompassing RRAR (SEQ ID NO: 99) furin cleavage site, and K⁹⁸⁶P and V⁹⁸⁷P consecutive substitutions are indicated.

FIG. 12. Single i.n. injection of LV::S_{ΔF2P} fully protects golden hamsters against SARS-CoV-2. (A) Timeline of the LV::S_{ΔF2P} prime-boost vaccination regimen and SARS-CoV-2 challenge in hamsters. (B) Serum anti-S_{CoV-2} IgG responses expressed as mean endpoint dilution titers, determined by ELISA. (C) Neutralization capacity of anti-S_{CoV-2} Abs, expressed as EC50 neutralizing titers, determined in the sera and lung homogenates of LV::S_{ΔF2P}-immunized hamsters. (D) Percentages of weight loss in LV::S_{ΔF2P}- or sham-vaccinated hamsters at 4 dpi. (E) Lung viral loads quantitated by total E or Esg qRT-PCR at 4 dpi. Statistical significance of the differences was evaluated by two tailed unpaired t test; *=p<0.0402, ****=p<0.0001. Red lines indicate the limit of detection of each assay.

FIG. 13. Largely reduced infection-driven lung inflammation in LV::S_{ΔF2P}-vaccinated hamsters. (A) Heatmap recapitulating relative log 2 fold changes in the expression of inflammation-related mediators in S_{ΔF2P}- or sham-vaccinated individuals, as analyzed at 4 dpi by use of RNA extracted from total lung homogenates and normalized versus samples from untreated controls. Six individual hamsters per group are shown in the heatmap. (B) Lung histological H&E analysis, as studies at 4 dpi.

FIG. 14. Large permissibility of the lungs and brain of K18-hACE2^{IP-THV} transgenic mice to SARS-CoV-2 replication. (A) Representative genotyping results from 15 N1 B6.K18-hACE2^{IP-THV} mice as performed by qPCR to determine their hACE2 gene copy number per genome. (B) Phenotyping of the same mice, inoculated i.n. with 0.3×10⁵ TCID₅₀ at the age of 5-7 wks and viral loads determination in their various organs at 3 dpi by conventional E-specific qRT-PCR. (C) Comparative permissibility of diverse organs from K18-hACE2^{IP-THV} and B6.K18-ACE2^{2PrImn/JAX} transgenic mice to SARS-CoV-2 replication, as determined at 3 dpi by conventional E-specific or sub-genomic Esg-specific qRT-PCR. The red line indicates the qRT-PCR limit of detection. Statistical significance of the difference was evaluated by Mann-Whitney test (*=p<0.01, **=p <0.00). (D) Comparative quantitation of hACE-2 mRNA in the lungs and brain of B6.K18-hACE2^{IP-THV} and B6.K18-ACE2^{2PrImn/JAX} transgenic mice. (E) Heatmap recapitulating log 2 fold change in cytokine and chemokine mRNA expression in the lungs or brain of B6.K18-hACE2^{IP-THV} and B6.K18-ACE2^{2PrImn/JAX} transgenic mice at 3 dpi. Data were normalized versus untreated controls.

FIG. 15. Vaccination with LV::S_{ΔF2P} protects both lungs and central nervous system from SARS-CoV-2 infection in K18-hACE2^{IP-THV} transgenic mice. (A) Timeline of prime-boost LV::S_{ΔF2P} vaccination and SARS-CoV-2 challenge in K18-hACE2^{IP-THV} mice. (B) Serum neutralization capacity of anti-S_{CoV-2} Abs in LV::S_{ΔF2P}-vaccinated mice. (C) Viral loads as determined in diverse organs at 3 dpi by use of conventional E-specific or sub-genomic Esg-specific qRT-PCR. The red line indicates the qRT-PCR detection limit. Statistical significance of the difference was evaluated by Mann-Whitney test (*=p<0.01, **=p<0.001). (D) Cytometric gating strategy determined to identify and quantify lung NK cells and neutrophils in the lungs of LV::S_{ΔF2P}- or sham-vaccinated and SARS-CoV-2-challenged K18-hACE2^{IP-THV} transgenic mice at 3 dpi. Percentages of NK and neutrophil subset were calculated versus total lung CD45+ cells. (E) Relative log 2 fold change in cytokine and chemokine mRNA expression in the brain of LV::S_{ΔF2P}- or sham-immunized and SARS-CoV-2-challenged K18-hACE2^{IP-THV} transgenic mice at 3 dpi. Data were normalized versus untreated controls. Statistical significance was evaluated by two tailed unpaired t test; *=p<0.05, **=p<0.01).

FIG. 16. Vaccination with LV::S_{ΔF2P} through i.n. route elicits full protection of CNS from SARS-CoV-2 infection. (A) Timeline of various LV::S_{ΔF2P} vaccination regimens and SARS-CoV-2 challenge in B6.K18-hACE2^{IP-THV} mice. (B) Viral loads in the brain at 3 dpi determined by conventional E-specific or sub-genomic Esg-specific qRT-PCR. The red line indicates the qRT-PCR detection limit. Statistical significance of the difference was evaluated by Mann-Whitney test (*=p<0.01). (C-D) Cytometric analysis at 3 dpi performed on cells extracted from pooled olfactory bulbs or brain of LV::S_{ΔF2P} i. m.-i.n. vaccinated and protected mice versus sham-vaccinated and unprotected mice. (C) Adaptive and (D) innate immune cells in the olfactory bulbs. (E) Innate immune cells in the brain.

FIG. 17: Maps of lentiviral plasmid encoding S_{FL}, S1-S2, S1, S_{2P}, S_{2P3F} S_{ΔF2P}

FIG. 18: Head to head comparison of the protective potential of ILV::S_{FL} or ILV::S_{ΔF2P} in C57BL/6 mice pre-treated with Ad5::hACE2 and challenged with SARS-CoV-2.C57BL/6 mice were primed i.m. and boosted i.n. as described in Example 1. The animals were challenged i.n. with SARS-CoV-2 and viral load was measured at 3 dpi. The results show a slight difference between the two compared LV-borne constructs that is not considered significant and should even disappear when assessed by a sub-genomic qRT-PCR measuring replicating virus.

FIG. 19: plasmid map for pFLAP K18-hACE2 WPRE

FIGS. 20 to 24: Sequences of pFlap-CMV-S-2019-nCoV-WPREm, pFlap-ieCMV-S2P-WPREm, pFlap-ieCMV-S2P3F-WPREm, pFlap-ieCMV-S2P-ΔF-WPREm, pFLAP K18-hACE2 WPRE and the transgene sequences.

FIG. 25. Full protective capacity of NILV::S_{CoV-2} against the Manaus P.1 SARS-CoV-2 variant. (A) Timeline of NILV::S_{CoV-2} i.m.-i.n. immunization and challenge with Manaus P.1 SARS-CoV-2 in B6.K18-hACE2^{IP-THV} mice (n=5/group). Brains and lungs were collected at 3 dpi. (B) Brain or lung viral RNA contents, determined by conventional E-specific or sub-genomic Esg-specific qRT-PCR at 3 dpi. Two mice out of the 5 sham-vaccinated mice did not have detectable viral load in the lungs despite a high viral in the brain and hACE2 mRNA expression level comparable to the other mice in the same group. (C) Neutralizing activity (EC50) of sera from individual NILV::S_{CoV-2}-vaccinated mice against pseudo-viruses harboring S_{CoV-2} from the ancestral Wuhan strain or D614G, B1.117, B1.351 or P.1 variants. Statistical significance was evaluated by Mann-Whitney test (*=p<0.05, **=p<0.01). Red asterisk (bottom) indicates significance with ancestral Wuhan, blue asterisk (middle) indicates significance with D614G variant, while orange asterisk (top) indicates significance with B1.117 variant. Statistical comparisons were made at the respective boosting timepoint.

FIG. 26. T-cell response, plays a major role in NILV::S_{CoV-2}-mediated protection against SARS-CoV-2. (A) Wild type or μMT KO mice (n=5-9/group) were injected by LV::S_{CoV-2} or sham following the time line shown in (FIG. 1A), then pretreated with Ad5::hACE2 4 days before the challenge with SARS-CoV-2 Wuhan strain. Lung viral RNA contents were determined at 3 dpi. Statistical significance of the differences was evaluated by Mann-Whitney test (**=p<0.01, ****=p<0.0001). (B) T-splenocyte responses in NILV::S_{CoV-2}-primed and -boosted C57BL/6 WT mice or sham controls, evaluated by IFN-γ ELISPOT using 15-mer peptides encompassing S_{CoV-2} MHC-I-restricted epitopes. (C) Representative dot plots of IFN-γ response by lung CD8⁺ T cells, after in vitro stimulation with the indicated S_{CoV-2}-derived peptides. (D) Cytometric strategy to detect lung CD8⁺ T central memory (Tcm, CD44⁺CD62L⁺CD69⁻), T effector memory (Tem, CD44⁺CD62L⁻CD69⁻) and T resident memory (Trm, CD44⁺CD62L⁻CD69⁺CD103⁺) (top) and representative percentages of these subsets in LV::S i.m.-i.n.-vaccinated or sham mice.

FIG. 27. Features of olfactive bulbs in the protected NILV::S_{CoV-2}- or unprotected sham-vaccinated K18-hACE2^{IP-THV} mice. Mice are those detailed in the FIG. 2. (A-B) CD3 immuno-histo-chemistry of an olfactory bulb from a NILV::S_{CoV-2} i.m.-i.n. vaccinated and protected mice or unprotected sham-vaccinated mice and representative results from these groups at 3 dpi with SARS-CoV-2 Wuhan. (C) Cytometric analysis of cells extracted from pooled olfactory bulbs from the same groups. (D-E) density of CD3 T cells as determined by immuno-histo-chemistry of an olfactory bulb from a NILV::S_{CoV-2} i.m.-i.n. vaccinated and protected mice or unprotected sham-vaccinated mice and representative results from these groups at 3 dpi with SARS-CoV-2 Manaus P.1. (E) Cytometric analysis of cells extracted from pooled olfactory bulbs from the same groups.

FIG. 28. Cross-sero-neutralization potential in mice primed and boosted with LV encoding for each Spike of concern. (A) Timeline of i.p.-i.p. immunization in C57BL/6 mice (n=5/group). (B) Scheme showing the sero-neutralization test used. (C) Neutralizing activity (EC50) of sera from individual vaccinated mice against pseudo-viruses harboring S_{CoV-2} from the ancestral Wuhan strain or D614G, B1.1.7, B1.351 or P.1 variants.

FIG. 29. Effect of Spike stabilization by K⁹⁸⁶P-V⁹⁸⁷P substitutions (2P) on (cross) neutralizing antibody activity. (A) Timeline of i.p.-i.p. immunization in C57BL/6 mice (n=5/group). (B) Neutralizing activity (EC50) of sera from individual vaccinated mice against pseudo-viruses harboring S_{CoV-2} from the ancestral Wuhan strain or D614G, B1.1.7, B1.351 or P.1 variants.

FIGS. 30-34: Sequences of pFlap-ieCMV-S-B1.1.7-WPREm, pFlap-ieCMV-S-B351-WPREm, pFlap-ieCMV-S-B351-2P-WPREm, pFlap-ieCMV-SFL-D614G-WPREm, pFlap-ieCMV-S-P1-WPREm and the transgene sequences.

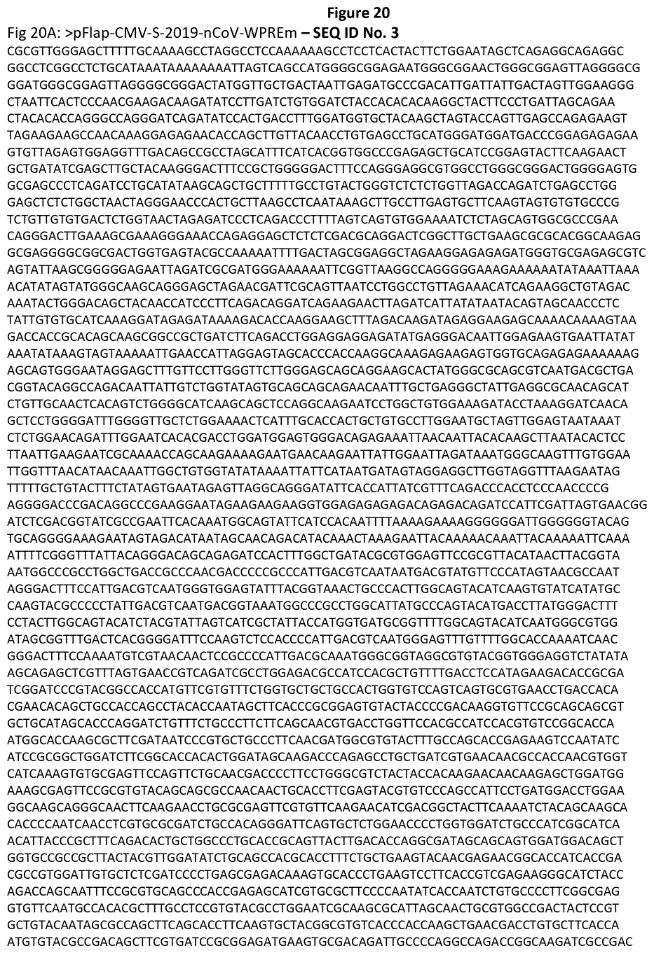

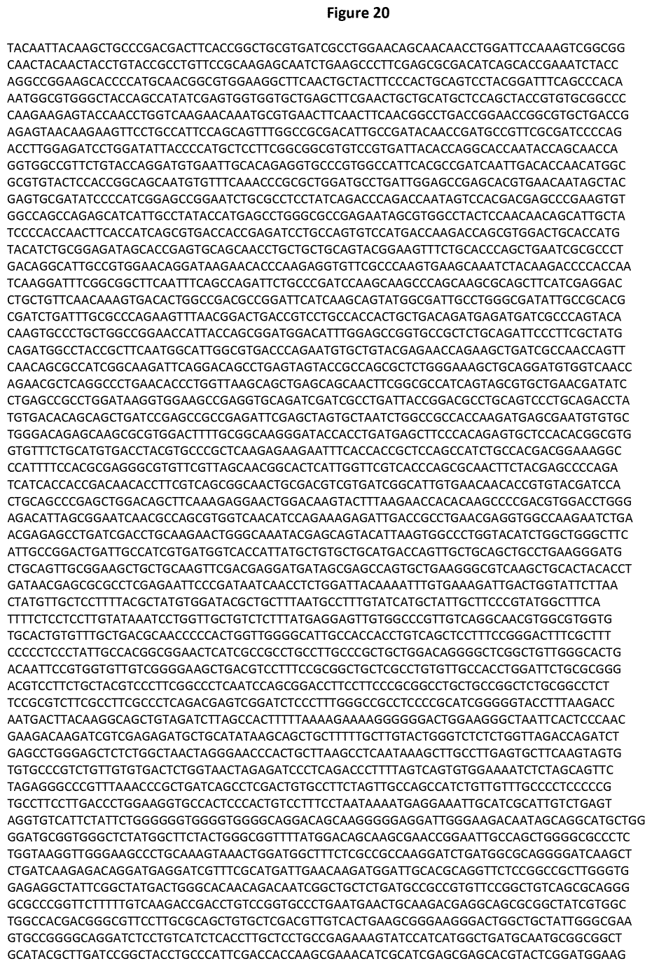

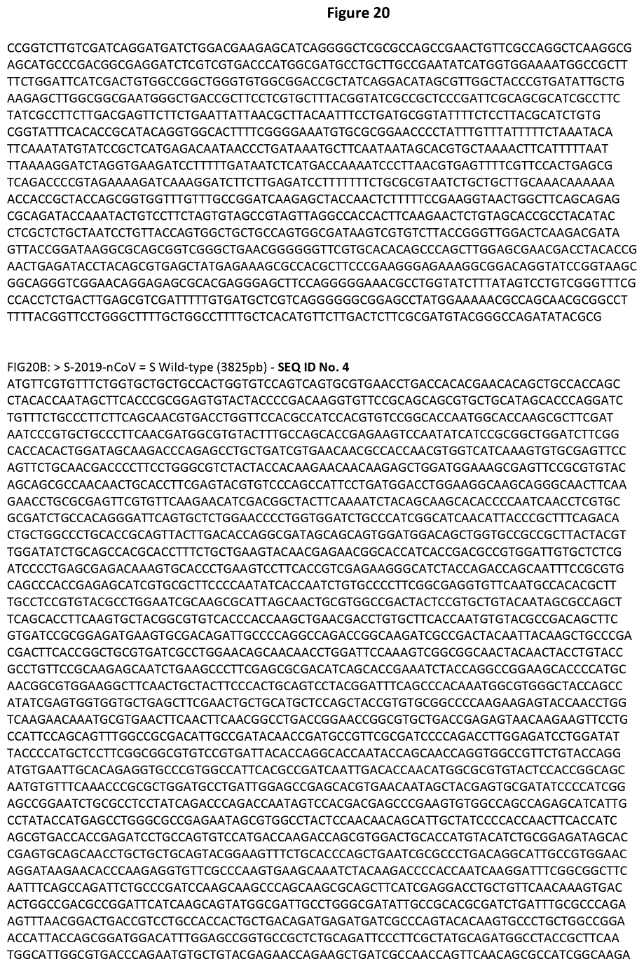

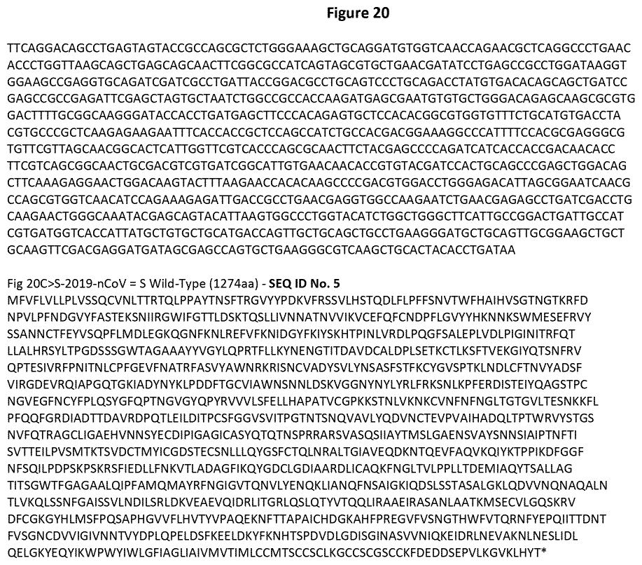

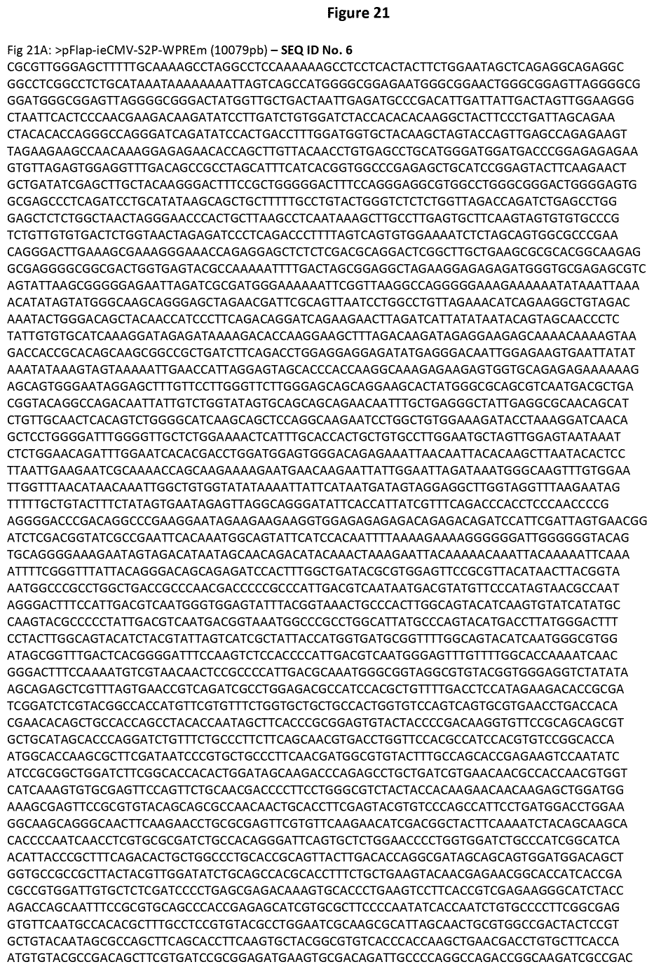

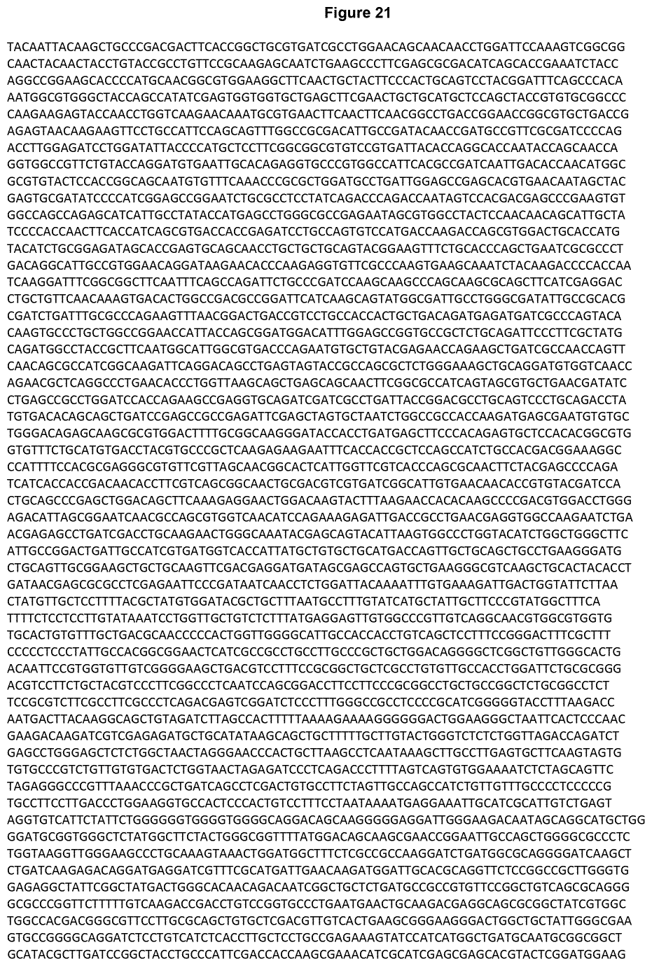

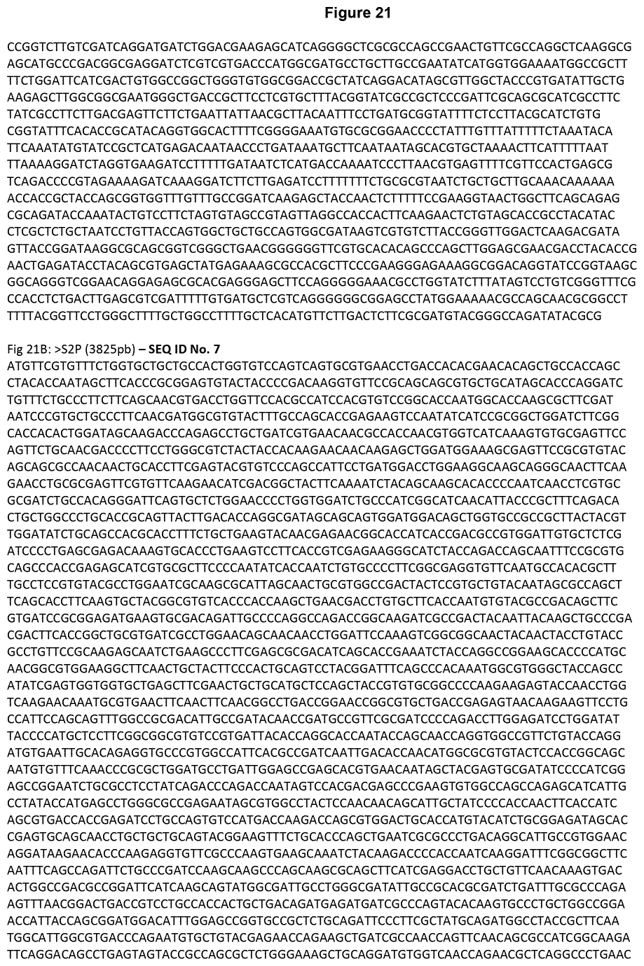

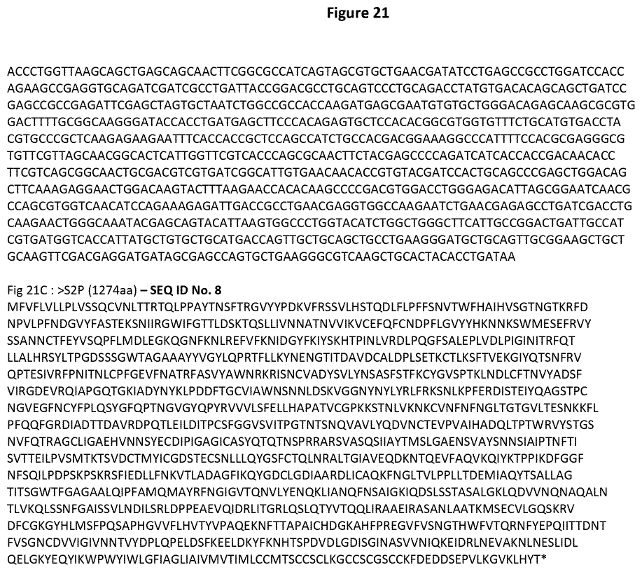

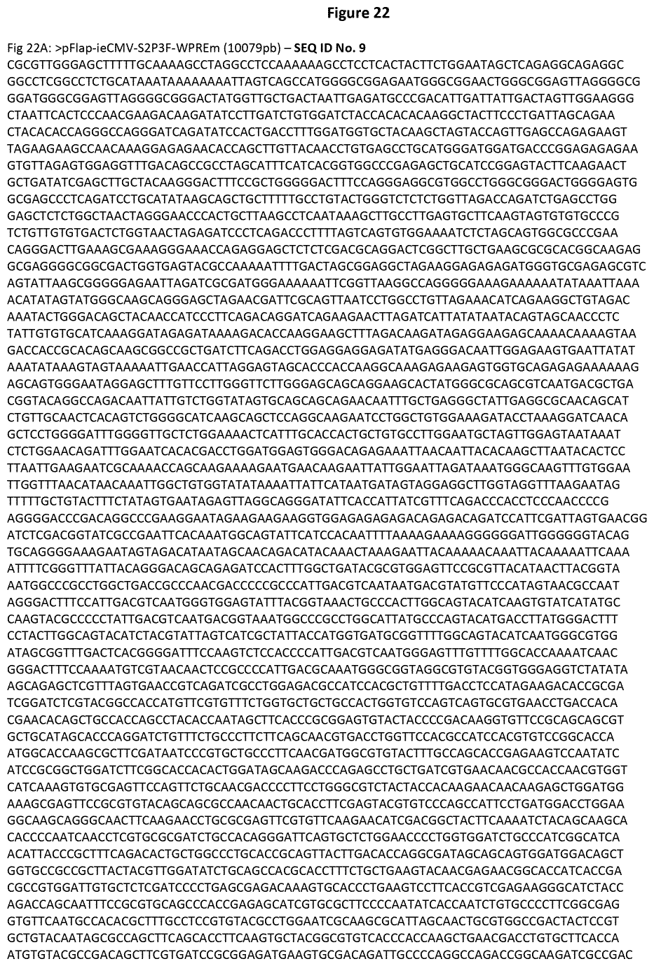

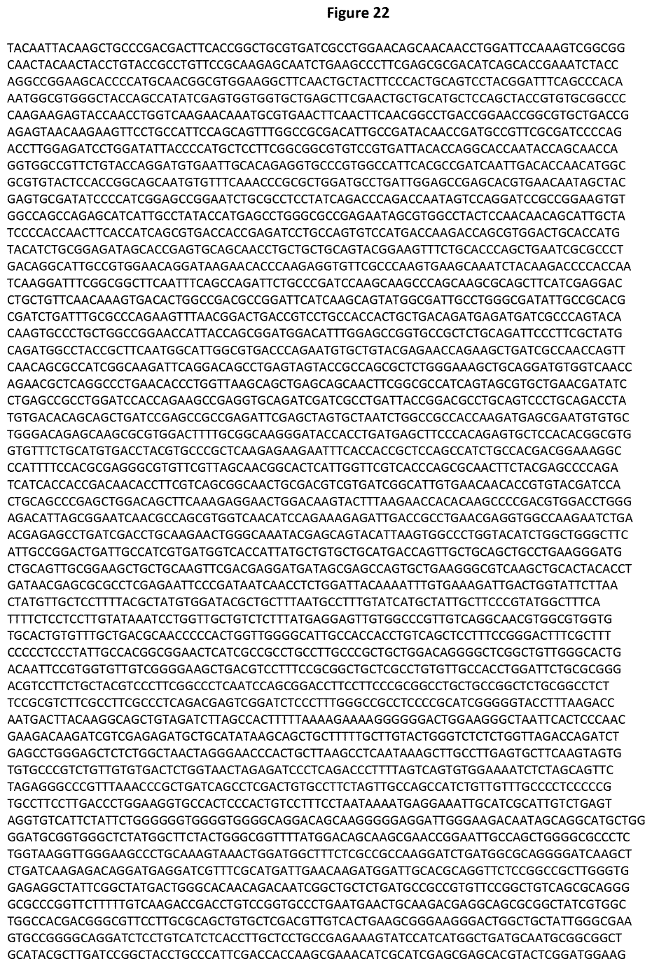

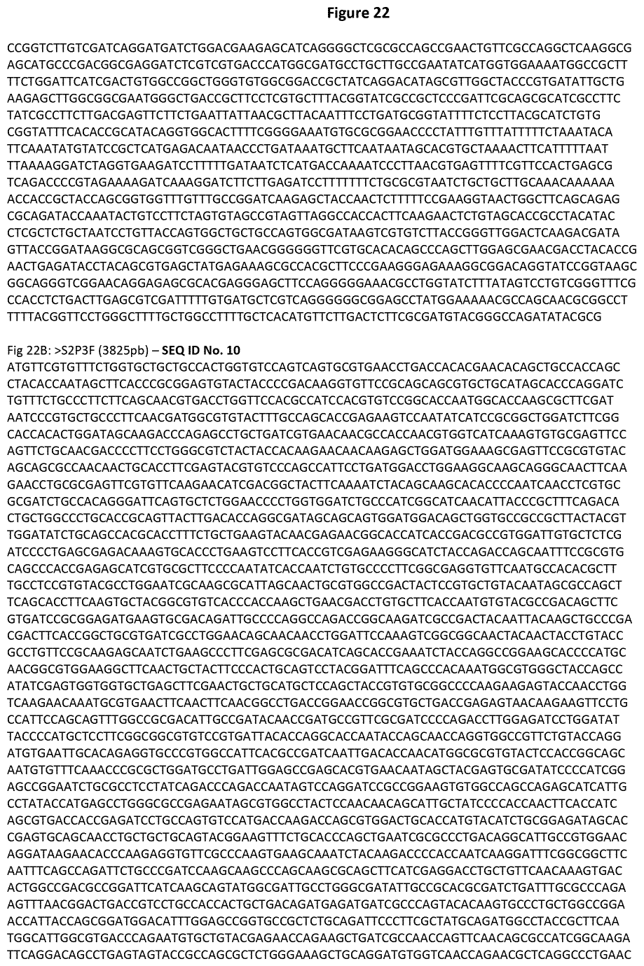

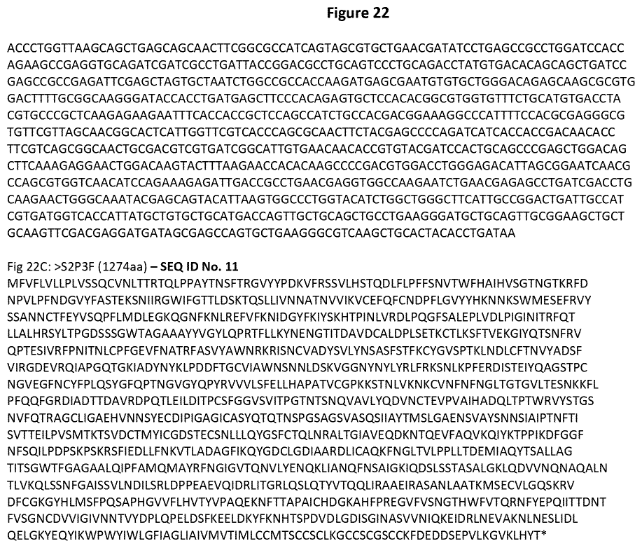

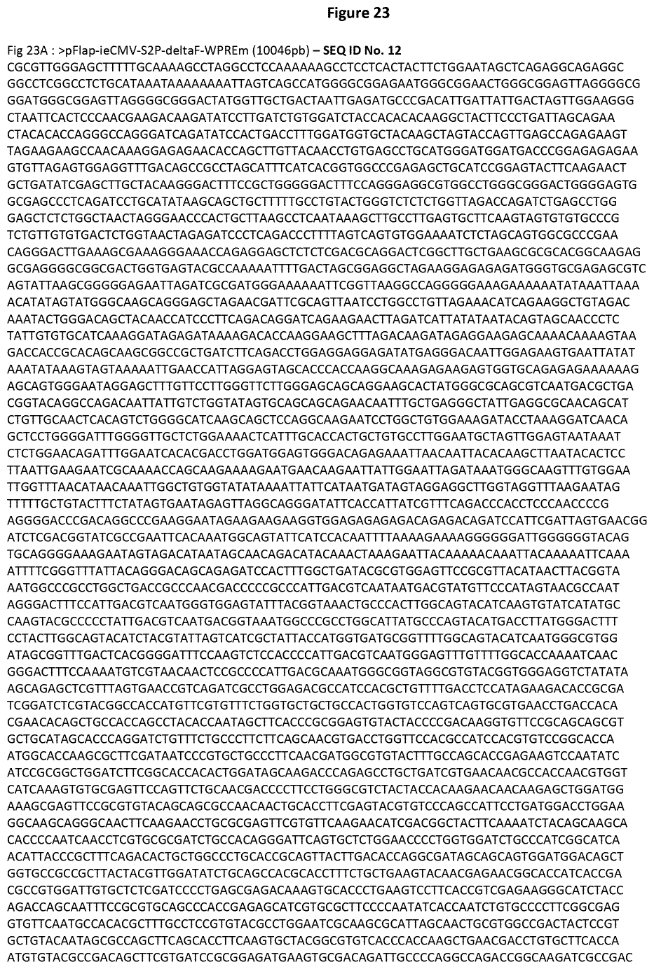

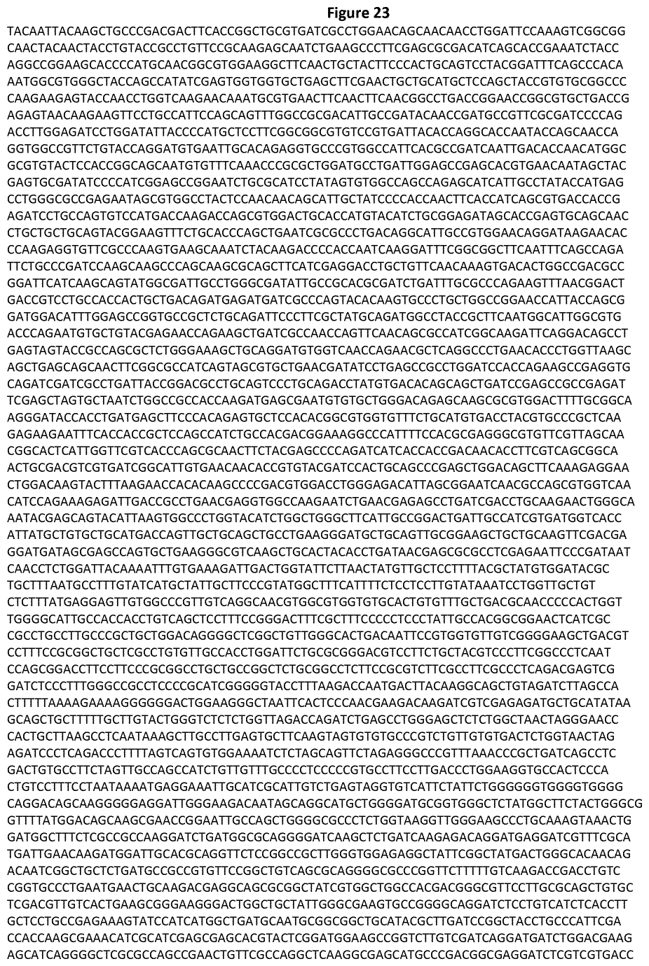

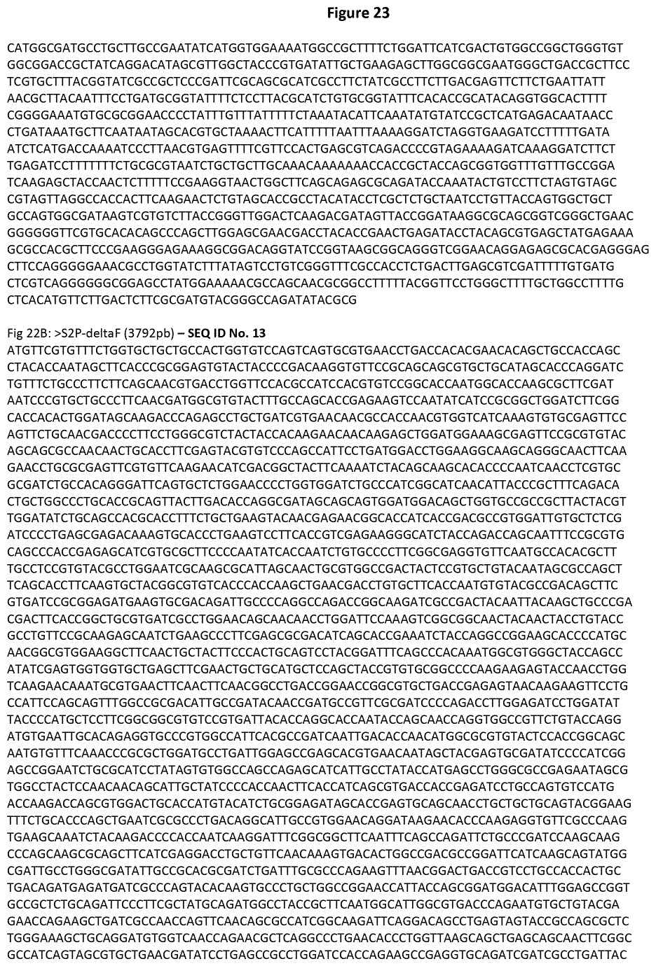

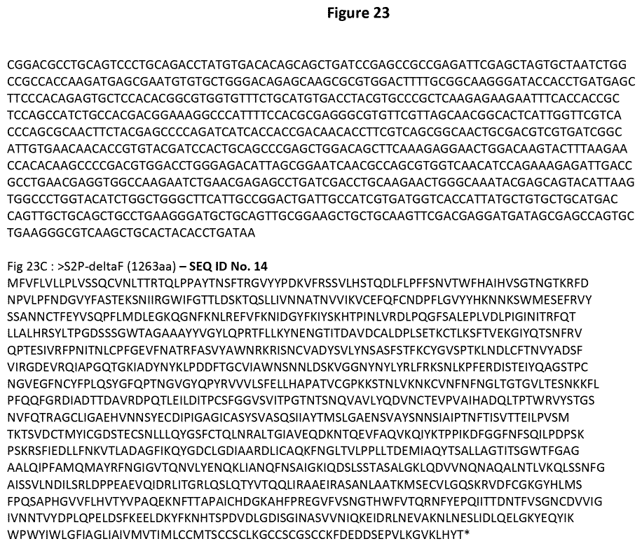

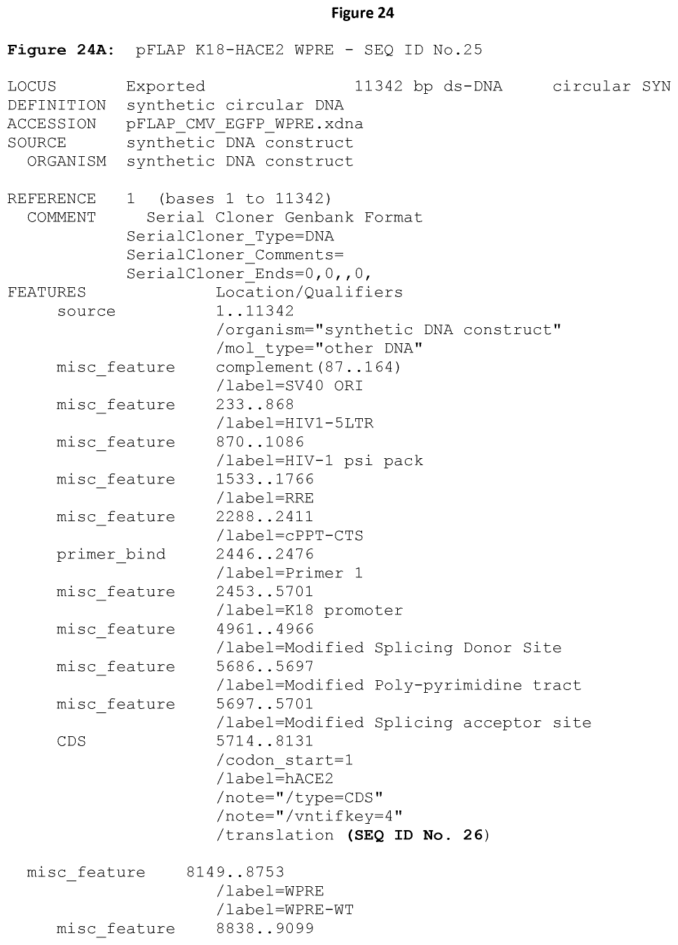

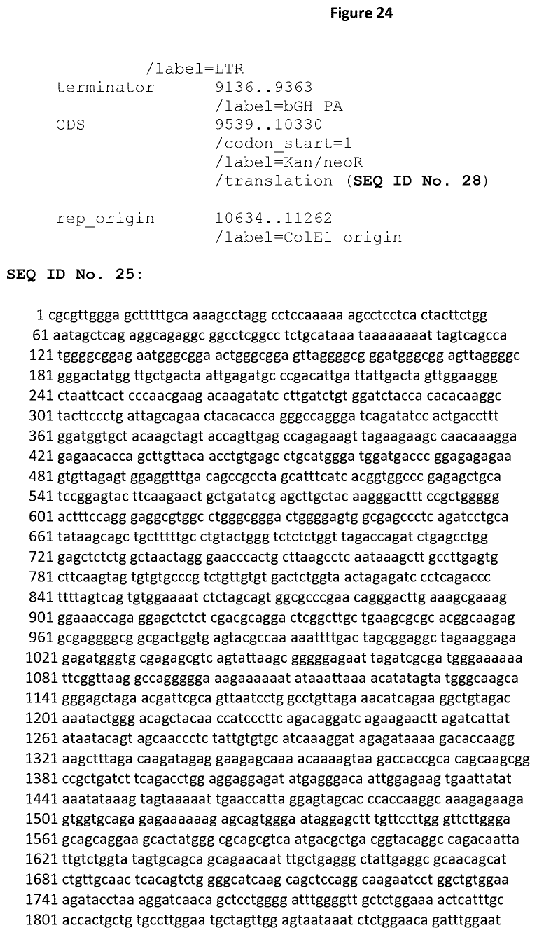

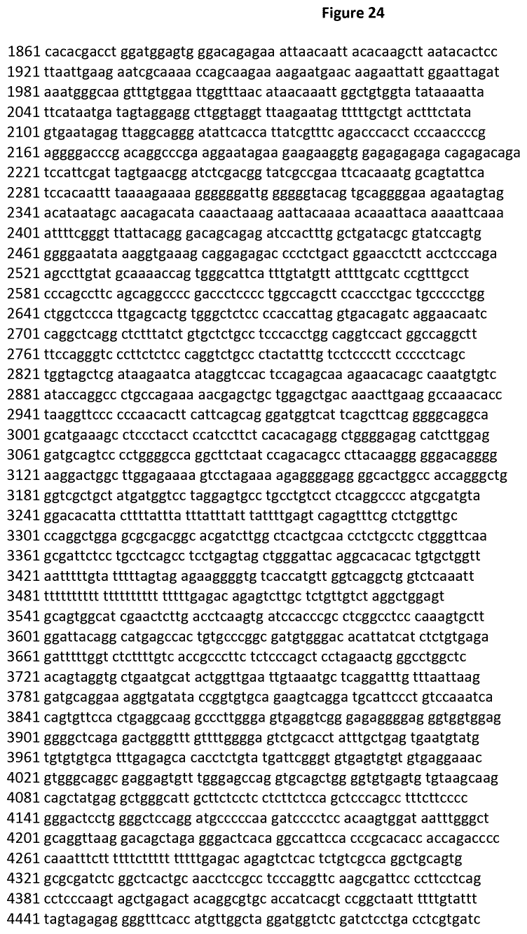

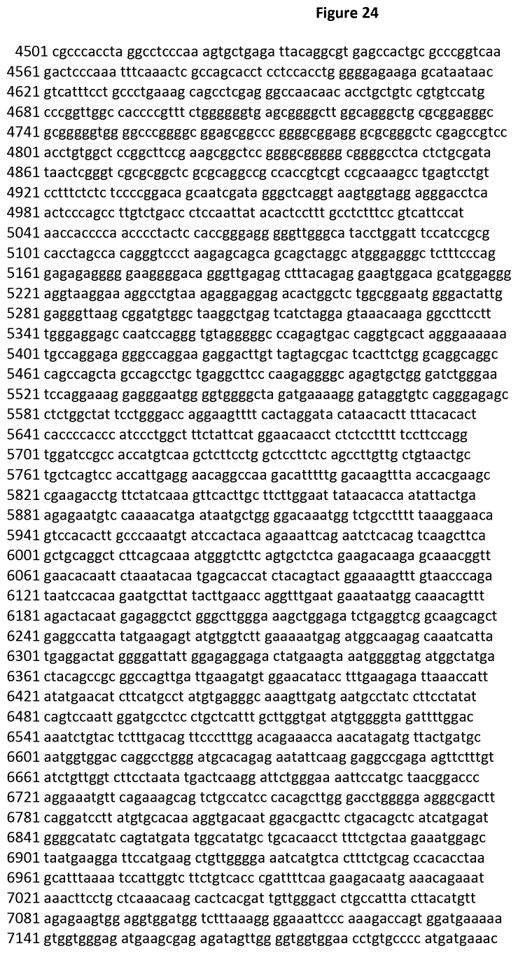

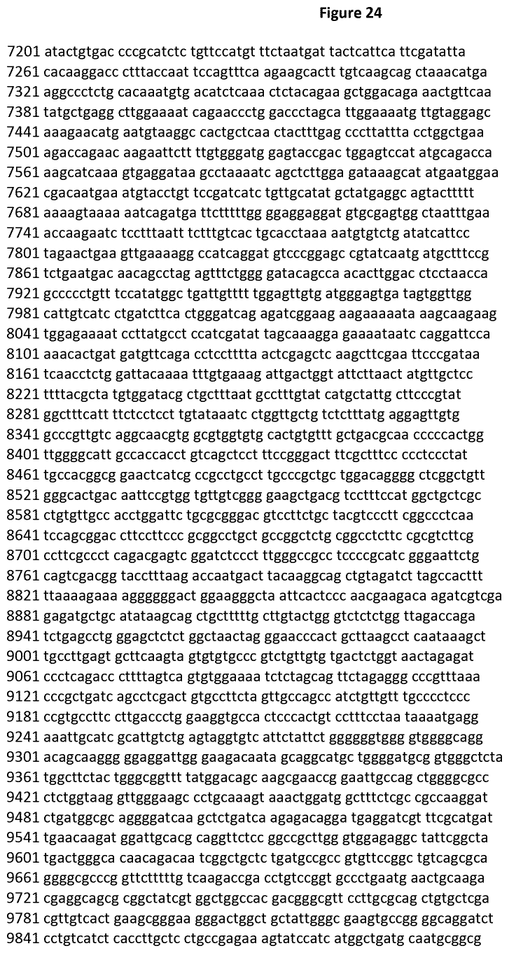

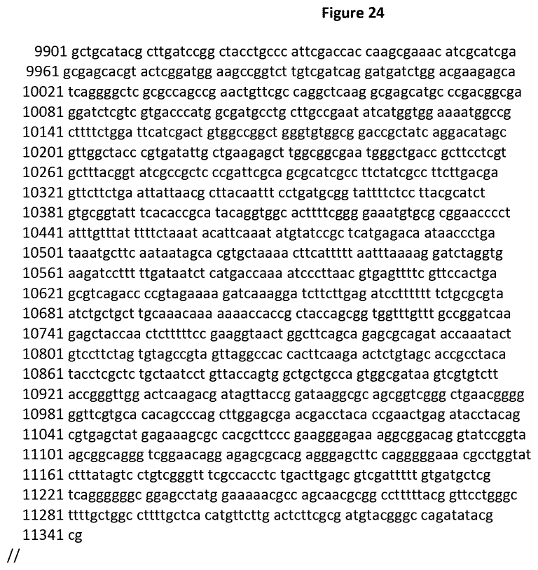

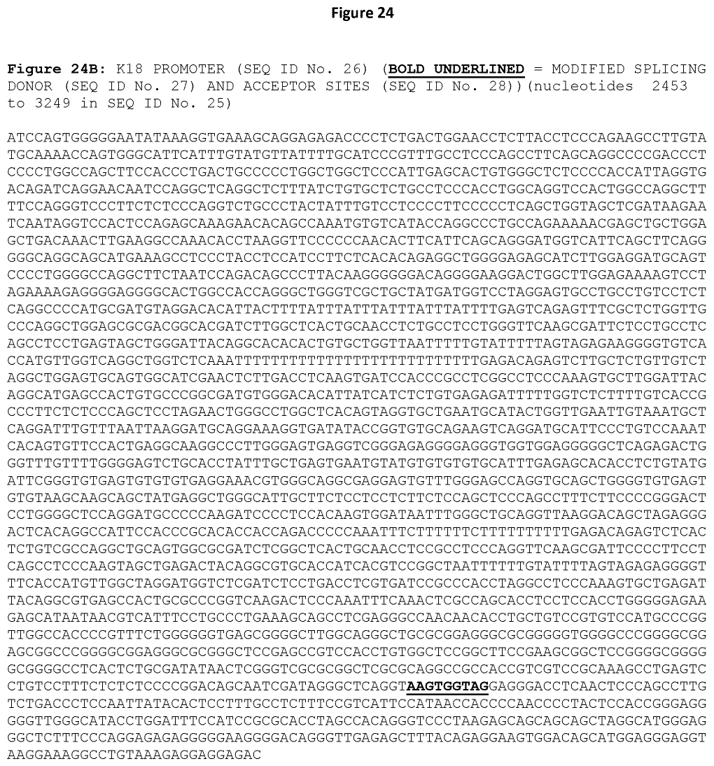

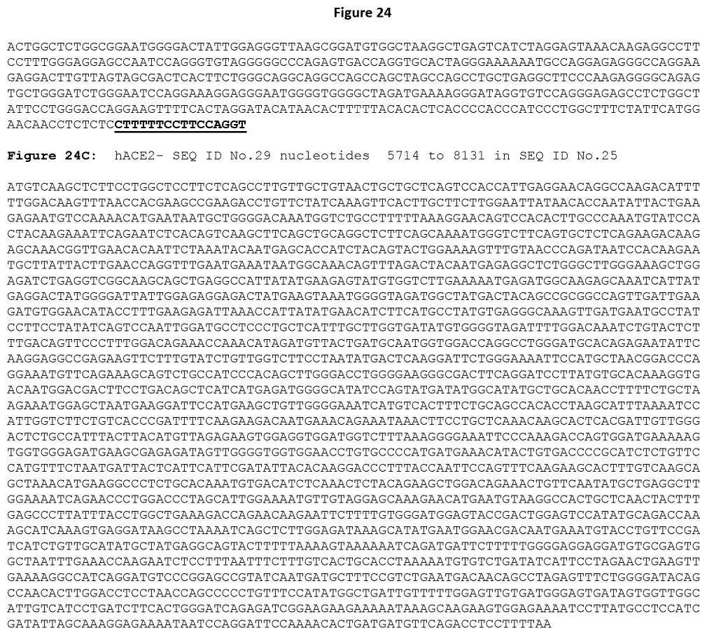

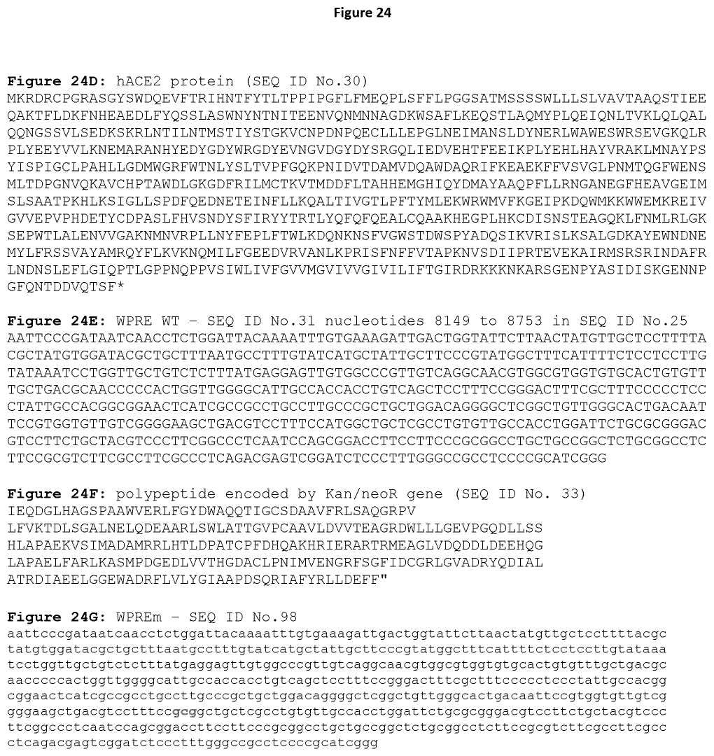

The sequences disclosed herein that are related to the transgene constructs are specified by their SEQ ID No. as follows:

| SEQ ID | | |
|---|---|---|
| No. | origin | Sequence disclosed in |
| 1 | Genbank: YP_009724390.1 | Text |
| 2 | Genbank: YP_009724390.1 | Text |
| 3 | pFlap-CMV-S-2019-nCoV-WPREm | FIG. 20 |
| 4 | SARS-COV-2 S (nt) | FIG. 20 |
| 5 | SARS-COV-2 S (aa) | FIG. 20 |
| 6 | pFlap-ieCMV-S2P-WPREm | FIG. 21 |
| 7 | S2P (nt) | FIG. 21 |
| 8 | S2P (aa) | FIG. 21 |
| 9 | pFlap-ieCMV-S2P3F-WPREm | FIG. 22 |
| 10 | S2P3F (nt) | FIG. 22 |
| 11 | S2P3F (aa) | FIG. 22 |
| 12 | pFlap-ieCMV-S2P-AF-WPREm | FIG. 23 |
| 13 | S2PAF (nt) | FIG. 23 |
| 14 | S2PAF(aa) | FIG. 23 |
| 15 | SARS-COV-2 S-peptide 61-75 | NVTWFHAIHVSGTNG |
| 16 | SARS-COV-2 S-peptide 536-550 | NKCVNFNFNGLTGTG |
| 17 | SARS-COV-2 S-peptide 576-590 | VRDPQTLEILDITPC |
| 18 | SARS-COV-2 S-peptide 441-455 | LDSKVGGNYNYLYRL |
| 19 | SARS-COV-2 S-peptide 671-685 | CASYQTQTNSPRRAR |
| 20 | SARS-COV-2 S-peptide 991-1005 | VQIDRLITGRLQSLQ |
| 21 | SARS-COV-2 S-peptide 256-275 | SGWTAGAAAYYVGYLQPRTF |
| 22 | SARS-COV-2 S-peptide 681-686 | PRRARS |
| 23 | SARS-COV-2 S-mutated peptide | PGSAGS |
| | 681-686 | |
| 24 | SARS-COV-2 S-peptide 675-685 | QTQTNSPRRAR |
| 25 | pFLAP K18-hACE2 WPRE | FIG. 24A |
| 26 | K18 promoter | FIG. 24A |
| 27 | Modified splicing donor site | AAGTGGTAG |
| 28 | Acceptor site | CTTTTTCCTTCCAGGT |
| 29 | hACE2 coding sequence(nt) | FIG. 24C |
| 30 | hACE2 protein | FIG. 24D |
| 31 | WPRE wild type (nt) | FIG. 24E |
| 98 | WPRE mutated (nt) | FIG. 24G |
| 33 | Polypeptide of the Kan/neoR gene | FIG. 24F |

### DETAILED DESCRIPTION

The inventions described herein are based in part on the potent vaccination strategy demonstrated in the examples. The examples demonstrate the utility of the vaccine strategy, which is based in certain embodiments on lentiviral vectors (LVs), able to induce neutralizing antibodies specific to the Spike glycoprotein (S) of SARS-CoV-2, the etiologic agent of CoronaVirus Disease 2019 (COVID-19). Among several LV encoding distinct variants of S, one encoding the full-length, membrane anchored S (LV::S_{FL}) and one encoding the mutated prefusion (an optionally stabilized) form such as in LV::S_{ΔF2P} (also designated LV::S2PΔF or LV::S2PDF or LV::S2PdeltaF) triggered high antibody titers in mice and hamsters, with substantial capacity to inhibit in vitro and in vivo viral invasion of host cells, expressing human Angiotensin-Converting Enzyme 2 (hACE2), the receptor for SARS-CoV-2 entry. S-specific T cells were also abundantly induced in LV::S_{FL}- or LV::S_{ΔF2P}-vaccinated individuals. In mice, in which the expression of hACE2 was induced by transduction of the respiratory tract cells by an adenoviral type 5 (Ad5) vector or by transgenesis with hACE2 vectorized by LV vector (B6.K18-hACE2^{IP-THV} mice), as well as in hamsters, substantial or full protective effect against pulmonary SARS-CoV-2 replication was afforded when LV::S_{FL} or LV::S_{ΔF2P} was used in systemic prime immunization, followed by intranasal mucosal boost/target. The conferred protection avoided pulmonary inflammation and prevented tissue damage. Besides, in B6.K18-hACE2^{IP-THV} mice with substantial brain permissibility to SARS-CoV-2 replication, protection was shown to extend to the brain and to CNS. The results presented demonstrate marked prophylactic effects of an LV-based vaccination strategy against SARS-CoV-2 in pre-clinical animal models and designate in particular the intranasal LV::S_{FL}-based immunization as a vigorous and promising vaccine approach against COVID-19. The i.n. boost after a systemic prime with LV-based vaccine is required to reach full protection of CNS in the developed transgenic model, which is a stringent model of SARS-CoV-2 infection with particularly high permissibility of brain to SARS-CoV-2 replication.

### A. SEVERE ACUTE RESPIRATORY SYNDROME BETA-CORONAVIRUS 2 SPIKE PROTEIN

Various aspects of this disclosure incorporate a SARS-CoV-2 S protein. In a preferred embodiment the SARS-CoV-2 S Protein comprises the following amino acid sequence (Genbank: YP_009724390.1; SEQ ID NO: 1):

| | |
|---|---|
| 1 | MFVFLVLLPL VSSQCVNLTT RTQLPPAYTN SFTRGVYYPD KVFRSSVLHS TQDLFLPFFS |
| 61 | NVTWFHAIHV SGTNGTKRFD NPVLPFNDGV YFASTEKSNI IRGWIFGTTL DSKTQSLLIV |
| 121 | NNATNVVIKV CEFQFCNDPF LGVYYHKNNK SWMESEFRVY SSANNCTFEY VSQPFLMDLE |
| 181 | GKQGNFKNLR EFVFKNIDGY FKIYSKHTPI NLVRDLPQGF SALEPLVDLP IGINITRFQT |
| 241 | LLALHRSYLT PGDSSSGWTA GAAAYYVGYL QPRTFLLKYN ENGTITDAVD CALDPLSETK |
| 301 | CTLKSFTVEK GIYQTSNERV QPTESIVRFP NITNLCPFGE VFNATRFASV YAWNRKRISN |
| 361 | CVADYSVLYN SASFSTFKCY GVSPTKLNDL CFTNVYADSF VIRGDEVRQI APGQTGKIAD |
| 421 | YNYKLPDDFT GCVIAWNSNN LDSKVGGNYN YLYRLFRKSN LKPFERDIST EIYQAGSTPC |
| 481 | NGVEGENCYF PLQSYGFQPT NGVGYQPYRV VVLSFELLHA PATVCGPKKS TNLVKNKCVN |
| 541 | FNFNGLTGTG VLTESNKKEL PFQQFGRDIA DTTDAVRDPQ TLEILDITPC SFGGVSVITP |
| 601 | GTNTSNQVAV LYQDVNCTEV PVAIHADQLT PTWRVYSTGS NVFQTRAGCL IGAEHVNNSY |
| 661 | ECDIPIGAGI CASYQTQTNS PRRARSVASQ SIIAYTMSLG AENSVAYSNN SIAIPTNFTI |
| 721 | SVTTEILPVS MTKTSVDCTM YICGDSTECS NLLLQYGSFC TQLNRALTGI AVEQDKNTQE |
| 781 | VFAQVKQIYK TPPIKDFGGF NFSQILPDPS KPSKRSFIED LLFNKVTLAD AGFIKQYGDC |
| 841 | LGDIAARDLI CAQKFNGLTV LPPLLTDEMI AQYTSALLAG TITSGWTFGA GAALQIPFAM |
| 901 | QMAYRENGIG VTQNVLYENQ KLIANQFNSA IGKIQDSLSS TASALGKLQD VVNQNAQALN |
| 961 | TLVKQLSSNF GAISSVLNDI LSRLDKVEAE VQIDRLITGR LQSLQTYVTQ QLIRAAEIRA |
| 1021 | SANLAATKMS ECVLGQSKRV DFCGKGYHLM SFPQSAPHGV VFLHVTYVPA QEKNFTTAPA |
| 1081 | ICHDGKAHFP REGVFVSNGT HWFVTQRNFY EPQIITTDNT FVSGNCDVVI GIVNNTVYDP |
| 1141 | LQPELDSFKE ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC GSCCKFDEDD |
| 1261 | SEPVLKGVKL HYT |

In another preferred embodiment the SARS-CoV-2 S protein consists of the amino acid sequence (Genbank: YP_009724390.1; SEQ ID NO: 1).

It is pointed out that, unless it would appear technically not applicable to the person skilled in the art, the definitions provided herein for the SARS-CoV-2 S protein or the polynucleotide encoding the SARS-CoV-2 S protein similarly apply to the derivatives or to the fragments of the SARS-CoV-2 S protein defined with respect to the sequences of SEQ ID No. 1 or respectively SEQ ID No.2.

In some embodiments the SARS-CoV-2 S protein comprises an amino acid sequence that is at least 90%, 91%, 92%, 93%, 94%, 95%, 96%, 97%, 98%, or 99% identical to SEQ ID NO: 1. Such SARS-CoV-2 S protein may qualify as a SARS-CoV-2 S protein derivative and/or as a SARS-CoV-2 S protein fragment if the obtained sequence is shorter than SEQ ID NO: 1. It may also be a sequence of a SARS-CoV-2 S protein expressed by a different strain of the virus than the originally identified isolate Wuhan-Hu-1 (accession number MN908947).

In some embodiments the SARS-CoV-2 S protein consists of an amino acid sequence that is at least 90%, 91%, 92%, 93%, 94%, 95%, 96%, 97%, 98%, or 99% identical to SEQ ID NO: 1. Such SARS-CoV-2 S protein may qualify as a SARS-CoV-2 S protein derivative and/or as a SARS-CoV-2 S protein fragment if the obtained sequence is shorter than SEQ ID NO: 1. It may also be a sequence of a SARS-CoV-2 S protein expressed by a different strain of the virus than the originally identified isolate Wuhan-Hu-1 (accession number MN908947). In some embodiments, the SARS-CoV-2 S protein derivative has an amino acid sequence at least 95% identical or at least 99% identical to SEQ ID NO:1. In one embodiment the SARS-CoV-2 spike protein derivative or fragment has the amino acid sequence of SEQ ID No. 8, SEQ ID No. 11, SEQ ID No. 108, SEQ ID No. 111, SEQ ID No. 114, SEQ ID No. 117, or SEQ ID No. 120, or the SARS-CoV-2 S protein derivative has an amino acid sequence at least 95% identical or at least 99% identical to S SEQ ID No. 8, SEQ ID No. 11, SEQ ID No. 108, SEQ ID No. 111, SEQ ID No. 114, SEQ ID No. 117, or SEQ ID No. 120 or the SARS-CoV-2 spike protein fragment has the amino acid sequence of SEQ ID No. 14 or the SARS-CoV-2 S protein derivative has an amino acid sequence at least 95% identical or at least 99% identical to SEQ ID NO: 14.

In some embodiments the SARS-CoV-2 S protein comprises an amino acid sequence that has 1, 2, 3, 4, 5, 6, 7, 8, 9 or 10 amino acid changes relative to SEQ ID NO: 1. In some embodiments the SARS-CoV-2 S protein comprises of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1. Such SARS-CoV-2 S protein may qualify as a SARS-CoV-2 S protein derivative and/or as a SARS-CoV-2 S protein fragment if the obtained sequence is shorter than SEQ ID NO: 1. It may also be a sequence of a SARS-CoV-2 S protein expressed by a different variant of the virus than the originally identified isolate Wuhan-Hu-1 (accession number MN908947).

In some embodiments the SARS-CoV-2 S protein consists of an amino acid sequence that has 1, 2, 3, 4, 5, 6, 7, 8, 9 or 10 amino acid changes relative to SEQ ID NO: 1. In some embodiments the SARS-CoV-2 S protein consist of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1 in particular no more than 10 amino acid changes at a single location in the protein. In some embodiments the SARS-CoV-2 S protein harbors mutation(s) such as those of the nucleotide sequence encoding S2PΔF or S2P3F In some embodiments a SARS-CoV-2 Spike protein comprises mutation(s) in the Receptor Binding Domain of the protein. In some embodiments the SARS-CoV-2 Spike protein harbors a substitution at residue 614 such as D614G or comprises such substitution. In some embodiments the SARS-CoV-2 Spike protein harbors mutation(s) identified in so-called variant SARS-CoV-2 VUI 2020 12/01 S protein i.e., mutations by substitution or deletion of amino acid residues of the Spike protein such as deletion 69-70, deletion 144, N501Y, substitutions A570D, D614G, P681H, T716I, S982A and D1118H. In some embodiments the SARS-CoV-2 Spike protein harbors mutation(s) that are present in SEQ ID No. 108, SEQ ID No. 111, SEQ ID No. 114, SEQ ID No. 117, or SEQ ID No. 120.

In a preferred embodiment the SARS-CoV-2 S protein is encoded by a nucleotide sequence that comprises nucleotides 21563 to 25384 of Genbank: NC_045512.2 (SEQ ID NO: 2):

| | |
|---|---|
| 21541 | atgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tcgttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tcccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatC ctttgcaacc tgaattagac tcattcaagg aggagttaga |
| 25021 | taaatatttt aagaatcata catcaccaga tcttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tCcaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataa |

In a preferred embodiment the SARS-CoV-2 S protein is encoded by a nucleotide sequence that consists of nucleotides 21563 to 25384 of Genbank: NC_045512.2 (SEQ ID NO: 2).

In some embodiments the SARS-CoV-2 S protein is encoded by a nucleotide sequence that is at least 60%, 70%, 80%, 85%, 90%, 91%, 92%, 93%, 94%, 95%, 96%, 97%, 98%, or 99% identical to SEQ ID NO: 2. Such SARS-CoV-2 S protein may qualify as a SARS-CoV-2 S protein derivative and/or as a SARS-CoV-2 S protein fragment if the nucleotide sequence having such defined percentage of identity is shorter than SEQ ID NO: 2. It may also be a sequence encoding a SARS-CoV-2 S protein which originates from a different strain of the virus than the originally identified isolate Wuhan-Hu-1 (accession number MN908947). In some embodiments the nucleotide sequence encoding the SARS-CoV-2 Spike protein harbors mutation(s) encompassing at least one non-synonymous mutation. In some embodiments the SARS-CoV-2 S protein is encoded by a nucleotide sequence that harbors mutation(s) such as those of the nucleotide sequence encoding S2PΔF or S2P3F. In some embodiments the nucleotide sequence encoding the SARS-CoV-2 Spike protein harbors a mutation at location 23403 in the sequence of SEQ ID No.2 wherein codon GGT is mutated, in particular substituted for codon GAT (corresponding to mutation at location 614, in particular to D614G substitution in the encoded protein). In some embodiments the nucleotide sequence is the sequence encoding the Spike protein of the so-called variant SARS-CoV-2 VUI 2020 12/01 wherein the Spike protein harbors multiple mutations by substitution or deletion of nucleotides wherein the mutations lead to the following changes in the amino acid residues of the encoded Spike protein: deletion 69-70, deletion 144, substitutions N501Y, A570D, D614G, P681H, T716I, S982A and D1118H.

In some embodiments the SARS-CoV-2 S protein is encoded by a nucleotide sequence that is codon-optimized, such as a codon optimized variant of SEQ ID NO: 2.

In some embodiments, the SARS-CoV-2 S protein comprises K986P and V987P amino acid substitutions.

In some embodiments, the SARS-CoV-2 S protein comprises a modification in which amino acids 681-686 are changed PRRARS (SEQ ID NO: 22) to PGSAGS (SEQ ID NO: 23).

In some embodiments, the SARS-CoV-2 S protein comprises a modification in which amino acids 675-685 (QTQTNSPRRAR (SEQ ID NO: 24)) are deleted.

### B. LENTIVIRAL VECTORS AND PSEUDOTYPED LENTIVIRAL VECTOR PARTICLES ENCODING A SEVERE ACUTE RESPIRATORY SYNDROME BETA-CORONAVIRUS 2 (SARS-COV-2) SPIKE (S) PROTEIN

Within the context of this invention, a “lentiviral vector” means a non-replicating vector for the transduction of a host cell with a transgene comprising cis-acting lentiviral RNA or DNA sequences, and requiring lentiviral proteins (e.g., Gag, Pol, and/or Env) that are provided in trans. The lentiviral vector lacks expression of functional Gag, Pol, and Env proteins. The lentiviral vector may be present in the form of an RNA or DNA molecule, depending on the stage of production or development of said retroviral vectors.

The lentiviral vector can be in the form of a recombinant DNA molecule, such as a plasmid. The lentiviral vector can be in the form of a lentiviral vector particle, such as an RNA molecule(s) within a complex of lentiviral other proteins. Typically, lentiviral particle vectors, which correspond to modified or recombinant lentivirus particles, comprise a genome which is composed of two copies of single-stranded RNA. These RNA sequences can be obtained by transcription from a double-stranded DNA sequence inserted into a host cell genome (proviral vector DNA) or can be obtained from the transient expression of plasmid DNA (plasmid vector DNA) in a transformed host cell.

The lentiviral vector particles may have the capacity for integration. As such, they contain a functional integrase protein. Alternatively, the lentiviral vector particles may have impaired or no capacity for integration. Non-integrating vector particles have one or more mutations that eliminate most or all of the integrating capacity of the lentiviral vector particles. For, example, a non-integrating vector particle can contain mutation(s) in the integrase encoded by the lentiviral pol gene that cause a reduction in integrating capacity. In contrast, an integrating vector particle comprises a functional integrase protein that does not contain any mutations that eliminate most or all of the integrating capacity of the lentiviral vector particles.

In some embodiments the lentiviral vector particles are integrative (ILV).

In some embodiments the lentiviral vector particles are non-integrative (NILV).

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FIV), which are modified to remove genetic determinants involved in pathogenicity and introduce new determinants useful for obtaining therapeutic effects. Preferably lentiviral vectors derive from HIV-1.

Such vectors are based on the separation of the cis- and trans-acting sequences. In order to generate replication-defective vectors, the trans-acting sequences (e.g., gag, pol, tat, rev, and env genes) can be deleted and replaced by an expression cassette encoding a transgene.

Efficient integration and replication in non-dividing cells generally requires the presence of two cis-acting sequences at the center of the lentiviral genome, the central polypurine tract (cPPT) and the central termination sequence (CTS). These lead to the formation of a triple-stranded DNA structure called the central DNA “flap”, which acts as a signal for uncoating of the pre-integration complex at the nuclear pore and efficient importation of the expression cassette into the nucleus of non-dividing cells, such as dendritic cells.

In one embodiment, the invention encompasses a lentiviral vector comprising a central polypurine tract and central termination sequence referred to as cPPT/CTS sequence as described, in particular, in the European patent application EP 2 169 073.

Further sequences are usually present in cis, such as the long terminal repeats (LTRs) that are involved in integration of the vector proviral DNA sequence into a host cell genome. Vectors may be obtained by mutating the LTR sequences, for instance, in domain U3 of said LTR (AU3) (Miyoshi H et al, 1998, J Virol. 72(10):8150-7; Zufferey et al., 1998, J Virol 72(12):9873-80).

In some embodiments the vector does not contain an enhancer. In some embodiments the lentiviral vector comprises LTR sequences, preferably with a mutated U3 region (ΔU3) removing promoter and enhancer sequences in the 3′ LTR.

The packaging sequence ψ (psi) can also be incorporated to help the encapsidation of the polynucleotide sequence into the vector particles (Kessler et al., 2007, Leukemia, 21(9):1859-74; Paschen et al., 2004, Cancer Immunol Immunother 12(6): 196-203).

In some embodiments, the invention encompasses a lentiviral vector comprising a lentiviral packaging sequence ψ (psi).

Further additional functional sequences, such as a transport RNA-binding site or primer binding site (PBS) or a Woodchuck PostTranscriptional Regulatory Element (WPRE) wild type or mutated (WPREm) a mutation being introduced to the start codon of protein X in WPRE to avoid expression of X protein peptide, can also be included in the lentiviral vector polynucleotide sequence, which in some embodiments allows for a more stable expression of the transgene in vivo.

In some embodiments, the lentiviral vector comprises a PBS. In one embodiment, the invention encompasses a lentiviral vector comprising a WPRE and/or an IRES.

In some embodiments, the lentiviral vector comprises at least one cPPT/CTS sequence, one ψ sequence, one (preferably 2) LTR sequence, and an expression cassette including a transgene under the transcriptional control of a cytomegalovirus (CMV) immediate-early promoter, a β2m promoter or a class I MHC promoter.

Methods of producing lentiviral vector particles and lentiviral vector particles are also provided. A lentiviral vector particle (or lentiviral particle vector) comprises a lentiviral vector in association with viral proteins. The vector may be an integrating vector (IL) (in particular for the preparation of transgenic mice as illustrated below) or may be a non-integrating vector (NIL) in particular for administration to human subject.

In some embodiments, the lentiviral vector particles encode a SARS-CoV-2 S protein or a derivative or fragment thereof according to any of the embodiments disclosed herein.

In some embodiments, the lentiviral vector particles encode a SARS-CoV-2 S protein or a derivative or fragment thereof that comprises or consists of an amino acid sequence selected from SEQ ID NOS: 1, 5, 8, 11, 14, 108, 111, 114, 117, and 120.

In some embodiments, the lentiviral vector particles encode a SARS-CoV-2 S protein or a derivative or fragment thereof that comprises or consists of the amino acid sequence of SEQ ID NO: 1.

In some embodiments, the lentiviral vector particles encode a SARS-CoV-2 S protein or a derivative or fragment thereof that comprises or consists of the amino acid sequence of SEQ ID NO: 5.

In some embodiments, the lentiviral vector particles encode a SARS-CoV-2 S protein or a derivative or fragment thereof that comprises or consists of the amino acid sequence of SEQ ID NO: 8.

In some embodiments, the lentiviral vector particles encode a SARS-CoV-2 S protein or a derivative or fragment thereof that comprises or consists of the amino acid sequence of SEQ ID NO: 11.

In some embodiments, the lentiviral vector particles encode a SARS-CoV-2 S protein or a derivative or fragment thereof that comprises or consists of the amino acid sequence of SEQ ID NO: 14.

In some embodiments, the lentiviral vector particles encode a SARS-CoV-2 S protein or a derivative or fragment thereof that comprises or consists of the amino acid sequence of SEQ ID NO: 108.

In some embodiments, the lentiviral vector particles encode a SARS-CoV-2 S protein or a derivative or fragment thereof that comprises or consists of the amino acid sequence of SEQ ID NO: 111.

In some embodiments, the lentiviral vector particles encode a SARS-CoV-2 S protein or a derivative or fragment thereof that comprises or consists of the amino acid sequence of SEQ ID NO: 114.

In some embodiments, the lentiviral vector particles encode a SARS-CoV-2 S protein or a derivative or fragment thereof that comprises or consists of the amino acid sequence of SEQ ID NO: 117.

In some embodiments, the lentiviral vector particles encode a SARS-CoV-2 S protein or a derivative or fragment thereof that comprises or consists of the amino acid sequence of SEQ ID NO: 120.

In some embodiments, the lentiviral vector particles encode a SARS-CoV-2 S protein or a derivative or fragment thereof that consists of the amino acid sequence Genbank: YP_009724390.1 (SEQ ID NO: 1).

In some embodiments, the lentiviral vector particles encode a SARS-CoV-2 S protein or a derivative or fragment thereof that comprises an amino acid sequence that is at least 90%, 91%, 92%, 93%, 94%, 95%, 96%, 97%, 98%, or 99% identical to SEQ ID NO: 1. The specific embodiments of such protein S derivative or fragment are also encompassed within these embodiments of the lentiviral vector particles.

In some embodiments, the lentiviral vector particles encode a SARS-CoV-2 S protein or a derivative or fragment thereof that comprises an amino acid sequence that is at least 90%, 91%, 92%, 93%, 94%, 95%, 96%, 97%, 98%, or 99% identical to SEQ ID NOS: 5, 8, 11, 14, 108, 111, 114, 117, or 120. The specific embodiments of such protein S derivative or fragment are also encompassed within these embodiments of the lentiviral vector particles.

In some embodiments, the lentiviral vector particles encode a SARS-CoV-2 S protein or a derivative or fragment thereof that consists of an amino acid sequence that is at least 90%, 91%, 92%, 93%, 94%, 95%, 96%, 97%, 98%, or 99% identical to SEQ ID NO: 1. The specific embodiments of such protein S derivative or fragment disclosed herein are also encompassed within these embodiments of the lentiviral vector particles.

In some embodiments, the lentiviral vector particles encode a SARS-CoV-2 S protein or a derivative or fragment thereof that consists of an amino acid sequence that is at least 90%, 91%, 92%, 93%, 94%, 95%, 96%, 97%, 98%, or 99% identical to SEQ ID NOS: 5, 8, 11, 14, 108, 111, 114, 117, or 120. The specific embodiments of such protein S derivative or fragment are also encompassed within these embodiments of the lentiviral vector particles.

In some embodiments, the lentiviral vector particles encode a SARS-CoV-2 S protein or a derivative or fragment thereof that comprises an amino acid sequence that has 1, 2, 3, 4, 5, 6, 7, 8, 9 or 10 amino acid changes relative to SEQ ID NO: 1. In some embodiments the SARS-CoV-2 S protein comprises of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In some embodiments, the lentiviral vector particles encode a SARS-CoV-2 S protein or a derivative or fragment thereof that comprises an amino acid sequence that has 1, 2, 3, 4, 5, 6, 7, 8, 9 or 10 amino acid changes relative to SEQ ID NOS: 5, 8, 11, 14, 108, 111, 114, 117, or 120. In some embodiments the SARS-CoV-2 S protein comprises of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In some embodiments, the lentiviral vector particles encode a SARS-CoV-2 S protein or a derivative or fragment thereof that consists of an amino acid sequence that has 1, 2, 3, 4, 5, 6, 7, 8, 9 or 10 amino acid changes relative to SEQ ID NO: 1. In some embodiments the SARS-CoV-2 S protein consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In some embodiments, the lentiviral vector particles encode a SARS-CoV-2 S protein or a derivative or fragment thereof that consists of an amino acid sequence that has 1, 2, 3, 4, 5, 6, 7, 8, 9 or 10 amino acid changes relative to SEQ ID NOS: 5, 8, 11, 14, 108, 111, 114, 117, or 120. In some embodiments the SARS-CoV-2 S protein consists of an amino acid sequence that has no more than 1, no more than 2, no more than 3, no more than 4, no more than 5, no more than 6, no more than 7, no more than 8, no more than 9 or no more than 10 amino acid changes relative to SEQ ID NO: 1.

In some embodiments the lentiviral vector particles encode a SARS-CoV-2 Spike protein that harbors mutation(s) such as those contained in S2PΔF (S2PdeltaF) or S2P3F protein derivatives.

In some embodiments the lentiviral vector particles encode a SARS-CoV-2 Spike protein that harbors a substitution at residue 614 such as D614G or that comprises such substitution. In some embodiments the lentiviral vector particles encode a SARS-CoV-2 Spike protein that harbors mutation(s) identified in so-called variant SARS-CoV-2 VUI 2020 12/01 S protein i.e., mutations by substitution or deletion of amino acid residues of the Spike protein such as deletion 69-70, deletion 144, N501Y, substitutions A570D, D614G, P681H, T716I, S982A and D1118H.

In some embodiments the lentiviral vector particles encode a SARS-CoV-2 S protein that is encoded by a nucleotide sequence that comprises SEQ ID NO: 2.

In some embodiments the lentiviral vector particles encode a SARS-CoV-2 S protein that is encoded by a nucleotide sequence that consists of nucleotides 21563 to 25384 of Genbank: NC_045512.2 (SEQ ID NO: 2).

In some embodiments the lentiviral vector particles comprise a nucleotide sequence that is at least 60%, 70%, 80%, 85%, 90%, 91%, 92%, 93%, 94%, 95%, 96%, 97%, 98%, or 99% identical to SEQ ID NO: 2.

In some embodiments the lentiviral vector particles encode a SARS-CoV-2 S protein that is encoded by the nucleotide sequence that harbors mutation(s) with respect to the sequence of SEQ ID NO: 2, wherein the mutation(s) encompass at least one non-synonymous mutation. In some embodiments the lentiviral vector particles encode a SARS-CoV-2 S protein whose nucleotide sequence harbors a mutation at location 23403 in the sequence of SEQ ID No.2 wherein codon GGT is mutated, in particular substituted for codon GAT (corresponding to mutation at location 614, in particular to D614G substitution in the encoded S protein of SEQ ID No.1). In some embodiments the lentiviral vector particles encode the Spike protein of the so-called variant SARS-CoV-2 VUI 2020 12/01 wherein the Spike protein harbors multiple mutations by substitution or deletion of nucleotides with respect to the sequence of SEQ ID No.2 and wherein the nucleotide mutations lead to the following changes in the amino acid residues of the encoded Spike protein: deletion 69-70, deletion 144, substitutions N501Y, A570D, D614G, P681H, T716I, S982A and D1118H.

In some embodiments the lentiviral vector particles comprise a nucleotide sequence that is codon-optimized, such as a codon optimized variant of SEQ ID NO: 2 or a codon optimized variant of the nucleotide sequence encoding the S2PΔF (S2PdeltaF) or the S2P3F derivatives.

In some embodiments the lentiviral vector particles comprise a nucleotide sequence that encodes a SARS-CoV-2 S protein that comprises K986P and V987P amino acid substitutions.

In some embodiments the lentiviral vector particles comprise a nucleotide sequence that encodes a SARS-CoV-2 S protein that comprises a modification in which amino acids 681-686 PRRARS (SEQ ID No.22) are changed to PGSAGS (SEQ ID No.23) such as in LV::S2P3F.

In some embodiments the lentiviral vector particles comprise a nucleotide sequence that encodes a SARS-CoV-2 S protein that comprises a modification in which amino acids 675-685 (QTQTNSPRRAR) (SEQ ID No.24) are deleted such as in LV::S2PΔF (LV::S2PdeltaF).

In some embodiments, the pseudotyped lentiviral vector particles comprise a polynucleotide selected from:
a polynucleotide encoding S2PΔF (S2PdeltaF) of SEQ ID No. 13 or a coding sequence having a nucleotide sequence at least 80% identical to SEQ ID No.13, in particular a coding sequence having a mutation, in particular a deletion, in the RBD,
a polynucleotide encoding S2P3F of SEQ ID No. 10 or a coding sequence having a nucleotide sequence at least 80% identical to SEQ ID No.10 having a mutation in the RBD, in particular wherein the coding sequence having a nucleotide sequence at least 80% identical to SEQ ID No.10 comprises mutations 986^{K→P} and 987^{V→P}.
a polynucleotide encoding S2P of SEQ ID No. 7 or a coding sequence having a nucleotide sequence at least 80% identical to SEQ ID No.7 having a mutation in the RBD,
a polynucleotide encoding SFL of SEQ ID No. 2 or a coding sequence having a nucleotide sequence at least 80% identical to SEQ ID No. 2 having a mutation in the RBD,
a polynucleotide encoding S-B1.1.7 of SEQ ID No. 107 or a coding sequence having a nucleotide sequence at least 80% identical to SEQ ID No. 107 having a mutation in the RBD,
a polynucleotide encoding S-B351 of SEQ ID No. 110 or a coding sequence having a nucleotide sequence at least 80% identical to SEQ ID No. 110 having a mutation in the RBD,
a polynucleotide encoding S-B1.1.7 S-B351-2P of SEQ ID No. 113 or a coding sequence having a nucleotide sequence at least 80% identical to SEQ ID No. 113 having a mutation in the RBD,
a polynucleotide encoding SFL-D614G of SEQ ID No. 116 or a coding sequence having a nucleotide sequence at least 80% identical to SEQ ID No. 116 having a mutation in the RBD, and
a polynucleotide encoding S-P1 of SEQ ID No. 119 or a coding sequence having a nucleotide sequence at least 80% identical to SEQ ID No. 119 having a mutation in the RBD.

In some embodiments, the lentiviral vector particle comprises HIV-1 Gag and Pol proteins. In some embodiments, the lentiviral vector particle comprises subtype D, especially HIV-1_{NDK}, Gag and Pol proteins.

According to some embodiments, the lentivector particles are obtained in a host cell transformed with a DNA plasmid.

Such a DNA plasmid can comprise:
bacterial origin of replication (ex: pUC ori);
antibiotic resistance gene (ex: KanR) for selection; and more particularly:
a lentiviral vector comprising at least one nucleic acid encoding a SARS-CoV-2 S protein or a derivative or fragment thereof, transcriptionally linked to a CMV promoter.

Such a method allows producing a recombinant vector particle according to the invention, comprising the following steps of:

i) transfecting a suitable host cell with a lentiviral vector;

ii) transfecting said host cell with a packaging plasmid vector, containing viral DNA sequences encoding at least structural and polymerase (+ integrase) activities of a retrovirus (preferably lentivirus); Such packaging plasmids are described in the art (Dull et al., 1998, J Virol, 72(11):8463-71; Zufferey et al., 1998, J Virol 72(12):9873-80).

iii) culturing said transfected host cell in order to obtain expression and packaging of said lentiviral vector into lentiviral vector particles; and

iv) harvesting the lentiviral vector particles resulting from the expression and packaging of step iii) in said cultured host cells.

For different reasons, in particular for administration to a human subject, it may be helpful to pseudotype the obtained retroviral particles, i.e. to add or replace specific particle envelope proteins. In some embodiments pseudotyping extends the spectrum of cell types that may be transduced while avoiding being the target of pre-existing immunity in human populations.

In order to pseudotype the retroviral particles of the invention, the host cell can be further transfected with one or several envelope DNA plasmid(s) encoding viral envelope protein(s), preferably a VSV-G envelope protein.

An appropriate host cell is preferably a human cultured cell line as, for example, a HEK cell line, such as a HEK293T line.

Alternatively, the method for producing the vector particle is carried out in a host cell, which genome has been stably transformed with one or more of the following components: a lentiviral vector DNA sequence, the packaging genes, and the envelope gene. Such a DNA sequence may be regarded as being similar to a proviral vector according to the invention, comprising an additional promoter to allow the transcription of the vector sequence and improve the particle production rate.

In a preferred embodiment, the host cell is further modified to be able to produce viral particle in a culture medium in a continuous manner, without the entire cells swelling or dying. One may refer to Strang et al., 2005, J Virol 79(3):1165-71; Relander et al., 2005, Mol Ther 11(3):452-9; Stewart et al., 2009, Gene Ther, 16(6):805-14; and Stuart et al., 2011, Hum gene Ther, with respect to such techniques for producing viral particles.

An object of the present invention consists of a host cell transformed with a lentiviral particle vector.

The lentiviral particle vectors can comprise the following elements, as previously defined:
cPPT/CTS polynucleotide sequence; and
a nucleic acid encoding a CAR under control of a 132m or MHCI promoter, and optionally one of the additional elements described above.

Preferably, the lentivector particles are in a dose of 10⁶, 2×10⁶, 5×10⁶, 10⁷, 2×10⁷, 5×10⁷, 10⁸, 2×10⁸, 5×10⁸, or 10⁹ TU.

This disclosure provides pseudotyped lentiviral vector particles bearing a SARS-CoV-2 S protein according to this disclosure. The lentivector can be integrative or non-integrative. The lentiviral vectors are pseudotyped lentiviral vectors (i.e. “lentiviral vector particles”) bearing a SARS-CoV-2 S protein.

The disclosure also provides an immunogenic composition comprising a lentiviral vector particle bearing a SARS-CoV-2 S protein according to this disclosure. All embodiments disclosed herein in relation to the lentiviral particles apply to the definition of the immunogenic composition.

In some embodiments, the immunogenic composition is for use in a method of prevention of infection of a human subject by SARS-CoV-2. In some embodiments, the immunogenic composition is for use in a method of protection against SARS-CoV-2 replication in a human subject at risk of being exposed to SARS-CoV-2 or infected by SARS-CoV-2. In some embodiments, the immunogenic composition is for use in a method of preventing development of symptoms or development of a disease associated with infection by SARS-CoV-2, such as COVID-19 in a human subject at risk of being exposed to SARS-CoV-2 or infected by SARS-CoV-2. In some embodiments, the immunogenic composition is for use in a method of preventing the onset of neurological outcome associated with infection by SARS-CoV-2 in a human subject at risk of being exposed to SARS-CoV-2 or infected by SARS-CoV-2. In some embodiments, the immunogenic composition is for use in a method of protecting the Central Nervous System (CNS) of a human subject at risk of being exposed to SARS-CoV-2 or infected by SARS-CoV-2. In some embodiments, in any of these applications for use in a method disclosed, the immunogenic composition may be administered to the subject as a prophylactic agent in an effective amount for elicitation of an immune response against SARS-CoV-2.

In some embodiment the immunogenic composition is for use in a method of protection of a human subject against SARS-CoV-2 infection or against development of the symptoms or the disease (COVID-19) associated with SARS-CoV-2 infection, wherein the subject is at risk of developing lung and/or CNS pathology. In particular the human subject is in need of immune protection of CNS from SARS-CoV-2 replication because he/she is affected with comorbid conditions, in particular comorbid conditions affecting the CNS.

The disclosure also provides a vaccine composition comprising a lentiviral vector particle bearing a SARS-CoV-2 S protein according to this disclosure and a carrier. In some embodiments the vaccine reduces the likelihood that a vaccinated subject, especially a human subject, will develop COVID-19. In some embodiments the reduction is by at least 30%, 40%, 50%, 60%, 70%, 80% or 90%. In some embodiments the vaccine reduces COVID-19 disease severity in a subject by at least 30%, 40%, 50%, 60%, 70%, 80%, or 90%. In some embodiments the reduction is by at least 30%, 40%, 50%, 60%, 70%, 80%, or 90%.

In some embodiments the vaccine provides protection against the infection by SARS-Cov-2, especially sterilizing protection. In some embodiments, the vaccine is for use in a method as disclosed herein in respect of the immunogenic composition.

The herein disclosed immunogenic composition and vaccine may be administered according to the administration route and administration regimen disclosed herein, in particular in accordance with the specific embodiments disclosed in C. below in particular in accordance with the illustrated embodiments.

### C. METHODS OF INDUCING AND/OR ACTIVATING A PROTECTIVE IMMUNE RESPONSE AGAINST SEVERE ACUTE RESPIRATORY SYNDROME BETA-CORONAVIRUS 2 (SARS-COV-2)

Also provided are methods of inducing or activating a protective immune response against Severe Acute Respiratory Syndrome beta-coronavirus 2 (SARS-CoV-2), comprising administering to the upper respiratory tract of a subject an effective amount of an agent that induces a protective immune response against SARS-CoV-2. In certain embodiments the agent that induces a protective immune response against SARS-CoV-2 is a pseudotyped lentiviral vector particle encoding a Severe Acute Respiratory Syndrome beta-coronavirus 2 (SARS-CoV-2) spike (S) protein or a derivative or fragment thereof. The disclosure of the methods herein is similarly applicable to the immunogenic composition for use in a method as disclosed in the present disclosure or to the vaccine for use in a method as disclosed in the present disclosure.

In some embodiments the agent is administered by nasal inhalation.

As used herein, “administered to the upper respiratory tract” includes any type of administration that results in delivery to the mucosa lining of the upper respiratory tract and includes in particular nasal administration. Administration to the upper respiratory tract includes without limitation aerosol inhalation, nasal instillation, nasal insufflation, and all combinations thereof. In some embodiments the administration is by aerosol inhalation. In some embodiments the administration is by nasal instillation. In some embodiments the administration is by nasal insufflation.

In some embodiments the treatment course consists of a single administration to the upper respiratory tract. In some embodiments the treatment course comprises a plurality of administrations to the upper respiratory tract. In some embodiments the treatment course comprises at least one administration to the upper respiratory tract and at least one administration outside of the respiratory tract. In some embodiments the treatment course comprises at least one priming administration via route outside of the respiratory tract followed by at least one boosting administration to the upper respiratory tract. The administration outside of the respiratory tract may be intramuscular, intradermal or subcutaneous. In some embodiments the treatment course comprises at least a prime/boost or a prime/target administration. In some embodiments the administration regimen comprises or consists of a prime administration outside of the upper respiratory tract, such as systemic (in particular intramuscular) administration and a boost or a target administration to the upper respiratory tract. The administered doses of the agent may be identical or may be different in the prime and boost/target administration steps, in particular may be higher for the administration to the upper respiratory tract. Details for the administration to the upper respiratory tract are provided below.

In a particular embodiment the lentiviral vector particles are LV::SFL, in particular NILV::SFL and the administration regimen consists in a systemic, especially i.m. prime and a boost to the upper respiratory tract, in particular by i.n. boost.

In a particular embodiment the lentiviral vector particles are LV::S_{prefusion}, in particular NILV::S_{prefusion}, such as LV::S2PΔF or NILV::S2PΔF, or LV::S2P3F or NI LV::S2P3F and the administration regimen consists in a systemic, especially i.m. prime and a boost to the upper respiratory tract, in particular by i.n. boost.

In some embodiments, the lentiviral vector particles comprise a polynucleotide selected from:
a polynucleotide encoding S2PΔF (S2PdeltaF) of SEQ ID No. 13 or a coding sequence having a nucleotide sequence at least 80% identical to SEQ ID No.13, in particular a coding sequence having a mutation, in particular a deletion, in the RBD,
a polynucleotide encoding S2P3F of SEQ ID No. 10 or a coding sequence having a nucleotide sequence at least 80% identical to SEQ ID No.10 having a mutation in the RBD, in particular wherein the coding sequence having a nucleotide sequence at least 80% identical to SEQ ID No.10 comprises mutations 986^{K→P} and 987^{V→P}.
a polynucleotide encoding S2P of SEQ ID No. 7 or a coding sequence having a nucleotide sequence at least 80% identical to SEQ ID No.7 having a mutation in the RBD,
a polynucleotide encoding SFL of SEQ ID No. 2 or a coding sequence having a nucleotide sequence at least 80% identical to SEQ ID No. 2 having a mutation in the RBD,
a polynucleotide encoding S-B1.1.7 of SEQ ID No. 107 or a coding sequence having a nucleotide sequence at least 80% identical to SEQ ID No. 107 having a mutation in the RBD,
a polynucleotide encoding S-B351 of SEQ ID No. 110 or a coding sequence having a nucleotide sequence at least 80% identical to SEQ ID No. 110 having a mutation in the RBD,
a polynucleotide encoding S-B1.1.7 S-B351-2P of SEQ ID No. 113 or a coding sequence having a nucleotide sequence at least 80% identical to SEQ ID No. 113 having a mutation in the RBD,
a polynucleotide encoding SFL-D614G of SEQ ID No. 116 or a coding sequence having a nucleotide sequence at least 80% identical to SEQ ID No. 116 having a mutation in the RBD, and
a polynucleotide encoding S-P1 of SEQ ID No. 119 or a coding sequence having a nucleotide sequence at least 80% identical to SEQ ID No. 119 having a mutation in the RBD.

In some embodiments the protective immune response comprises production of SARS-CoV-2 neutralizing antibodies in the subject. In some embodiments the neutralizing antibodies comprise IgG antibodies. In some embodiments the protective immune response comprises production of SARS-CoV-2 S-specific T cells in the subject. In some embodiments the SARS-CoV-2 S-specific T cells comprise CD4⁺ T cells. In some embodiments the SARS-CoV-2 S-specific T cells comprise CD8⁺ T cells. In some embodiments the SARS-CoV-2 S-specific T cells comprise CD4⁺ T cells and CD8⁺ T cells. In some embodiments the SARS-CoV-2 S-specific T cells comprise lung CD8⁺ T cells. In some embodiments the SARS-CoV-2 S-specific T cells comprise IFN-γ-producing T-cells. In some embodiments the SARS-CoV-2 S-specific T cells comprise T cells with an effector memory (Tem) and/or resident memory (Trm) phenotype. In some embodiments the SARS-CoV-2 S-specific T cells are recruited to the olfactory bulb. In some embodiments the protective immune response reduces the development of at least one symptom of a SARS-CoV-2 infection. In some embodiments the protective immune response reduces the time period during which an infected subject suffers from at least one symptom of a SARS-CoV-2 infection. In some embodiments the protective immune response reduces the likelihood of developing SARS-CoV-2 infection-related inflammation in the subject.

In various embodiments, the pseudotyped lentiviral vector particle may encode any Severe Acute Respiratory Syndrome beta-coronavirus 2 (SARS-CoV-2) spike (S) protein or a derivative or fragment thereof that is disclosed herein in the above embodiments relating to the description of the lentiviral vector particles.

In some embodiments the SARS-CoV-2 S protein derivative has an amino acid sequence at least 95% identical to SEQ ID NO: 1. In some embodiments the SARS-CoV-2 S protein derivative is expressed from a coding sequence having a nucleotide sequence at least 80% identical to SEQ ID NO: 2. In some embodiments the SARS-CoV-2 S protein fragment comprises a peptide selected from peptide 61-75 (NVTWFHAIHVSGTNG (SEQ ID NO: 15)), peptide 536-550 (NKCVNFNFNGLTGTG (SEQ ID NO: 16)) and peptide 576-590 (VRDPQTLEILDITPC (SEQ ID NO: 17)). In some embodiments the SARS-CoV-2 S derivative or fragment thereof comprises an amino acid modification relative to SEQ ID NO: 1, the modification selected from: (i) 986^{K→P} and 987^{V→P}, (ii) 681^{PRRARS}686 (SEQ ID NO: 22)→681^{PGSAGS}686 (SEQ ID NO: 23), and (iii) 986^{K→P}, 987^{V→P}, and 675^{QTQTNSPRRAR}685 (SEQ ID NO: 24) deletion. In some embodiments the Severe Acute Respiratory Syndrome beta-coronavirus 2 (SARS-CoV-2) spike (S) protein or a derivative or fragment thereof comprises or consists of an amino acid sequence selected from SEQ ID NOS: 1, 5, 8, 11, 14, 108, 111, 114, 117, and 120.

In some embodiments the administered lentiviral vector particle is integrative. In some embodiments the administered lentiviral vector particle is nonintegrative. In some embodiments the administered nonintegrative lentiviral particle comprises a D64V mutation in an integrase coding sequence. In some embodiments the administered lentiviral vector particle is pseudotyped with Vesicular Stomatitis Virus envelop Glycoprotein (VSV-G). In some embodiments the lentiviral vector particle is administered as a vaccine formulation comprising the lentiviral vector particle and a pharmaceutically acceptable carrier.

In some embodiments, the lentivector contains a promoter that drives high expression of the Severe Acute Respiratory Syndrome beta-coronavirus 2 (SARS-CoV-2) spike (S) protein or a derivative or fragment thereof, and drives expression in sufficient quantity for elimination by the induced immune response. In some embodiments, the promoter lacks an enhancer element to avoid insertional effects.

In some embodiments, at least 95%, 99%, 99.9%, or 99.99% of the lentiviral DNA integrated in cells of a mouse or hamster animal model at day 4 after administration is eliminated by day 21 after administration.

In some embodiments, the lentivector particles are in a dose of 10⁶, 2×10⁶, 5×10⁶, 10⁷, 2×10⁷, 5×10⁷, 10⁸, 2×10⁸, 5×10⁸, or 10⁹ TU.

The immune response induced by the lentiviral vector can be a B cell response, a CD4⁺ T cell response, and/or a CD8⁺ T cell response.

The present invention thus provides vectors that are useful as a medicament or vaccine, particularly for administration to the upper respiratory tract.

The disclosed lentiviral vectors have the ability to induce, improve, or in general be associated with the occurrence of a B cell response, a CD4⁺ T cell response, and/or a CD8⁺ T cell response, including a memory CTL response.

In some embodiments the lentiviral vector is used in combination with adjuvants, other immunogenic compositions, and/or any other therapeutic treatment.

According to some embodiments the immunogenic compositions as defined or illustrated herein are for use to induce a protective immune response against SARS-CoV-2 in the upper respiratory tract and/or in the brain against SARS-CoV-2 of a subject.

According to some embodiments the immunogenic compositions are for use to induce a cross protective immune response of lungs and brain against ancestral including SARS-CoV-2 selected from the group of SARS-CoV-2 Wuhan strain, SARS-CoV-2 D614G strain and SARS-CoV-2 B1.117 strain and against emerging SARS-CoV-2 variants such as SARS-CoV-2 P.1 variant, by eliciting B and T cell-responses.

According to some embodiments the immunogenic compositions are for use as defined herein and are characterized in that the dosage form or the pseudotyped lentiviral particle comprises pseudotyped lentiviral particles as defined herein wherein the pseudotyped lentiviral particles are non-integrative.

In some embodiments, these immunogenic compositions are for use to elicit a protective immune response against SARS-CoV-2 wherein the response elicits SARS-CoV-2 S-specific T cells, in particular SARS-CoV-2 S-specific T cells that comprise lung CD8⁺ T cells and/or IFN-γ-producing T-cells.

According to some embodiments the immunogenic compositions are for use to elicit a protective immune response against SARS-CoV-2 wherein the response elicits CD8⁺ T cells that comprise T cells with an effector memory (Tem) and/or resident memory (Trm) phenotype.

According to some embodiments the immunogenic compositions are for use as defined herein, the SARS-CoV-2 S-specific T cells are recruited to the olfactory bulb.

According to some embodiments the immunogenic compositions for use according to the invention are characterized in that the Severe Acute Respiratory Syndrome beta-coronavirus 2 (SARS-CoV-2) spike (S) protein or a derivative or fragment thereof comprises or consists of an amino acid sequence selected from SEQ ID NOS: 1, 5, 8, 11, 14, 108, 111, 114, 117, and 120.

According to some embodiments the immunogenic compositions are for use to prevent or to alleviate SARS-CoV-2 infection-related inflammation in the subject.

### D. DOSAGE FORMS FOR ADMINISTRATION TO THE UPPER RESPIRATORY TRACT

The immunogenic compositions of the disclosure may be provided in a dosage form suitable for administration to the upper respiratory tract of a subject. Appropriate formulations are known in the art. In some embodiments the dosage form is adapted for aerosol inhalation. In some embodiments the dosage form is adapted for nasal instillation. In some embodiments the nasal dosage form is adapted for nasal insufflation. In some embodiments the dosage form is aliquoted in a single dose. In some embodiments the dosage form is packaged in a single dose.

### E. KITS

Also provided are kits suitable for use in practicing a method disclosed herein. In some embodiments the kit comprises a dosage form for administration to the upper respiratory tract of a subject of the pseudotyped lentiviral vector particle encoding a SARS-CoV-2 S protein or a derivative or fragment thereof according to this disclosure, and an applicator. In some embodiments the applicator is an applicator for aerosol inhalation. In some embodiments the applicator is an applicator for nasal instillation. In some embodiments the applicator is an applicator for nasal insufflation. Suitable examples of each are known in the art and may be used.

### F. LENTIVIRAL VECTORS

Also provided are novel and nonobvious lentiviral vectors and plasmids for creating the same. The LV and the plasmids encode a Severe Acute Respiratory Syndrome beta-coronavirus 2 (SARS-CoV-2) spike (S) protein or a derivative or fragment thereof.

Having thus described different embodiments of the present invention, it should be noted by those skilled in the art that the disclosures herein are exemplary only and that various other alternatives, adaptations, and modifications may be made within the scope of the present invention. Accordingly, the present invention is not limited to the specific embodiments as illustrated herein.

### G. EXAMPLES

### Example 1: Intranasal Vaccination with LV Against SARS-Cov-2 in Preclinical Animal Models of Golden Hamster and Mice Treated to Express Human ACE2

### Example 1.1: Materials and Methods

1. 1.1 Construction of Transfer pFLAP Plasmids Coding SFL, S1-S2, or S1 Derived from SCoV-2.

codon-optimized full-length S (1-1273) sequence was amplified from pMK-RQ_S-2019-nCoV and inserted between BamHI and XhoI sites of pFlap-ieCMV-WPREm. Sequences encoding for S1-S2 (1-1211) or S1 (1-681) were amplified by PCR from the pFlap-ieCMV-SFL-WPREm plasmid and sub-cloned into pFlap-ieCMV-WPREm between the BamHI and XhoI restriction sites. Each of the PCR products were inserted between the native human ieCMV promoter and a mutated WPRE (Woodchuck Posttranscriptional Regulatory Element) sequence, where a mutation was introduced to the start codon of protein X in WPRE to avoid expression of X protein peptide. Plasmids were amplified in Escherichia coli DH5a in Lysogeny Broth (LB) supplemented with 50 μg/ml of kanamycin, purified using the NucleoBond Xtra Maxi EF Kit (Macherey Nagel) and resuspended in Tris-EDTA Endotoxin-Free (TE-EF) buffer overnight. The plasmid was quantified with a NanoDrop 2000c spectrophotometer (Thermo Scientific), adjusted to 1 μg/μl in TE-EF buffer, aliquoted and stored at −20° C. The plasmid DNA was verified by (i) diagnostic check with restriction digestion, and (ii) sequencing the region proximal to the transgene insertion sites.

1. 1.2 Production and Titration of LV Vectors

Non-replicative integrative LV vectors were produced in Human Embryonic Kidney (HEK)-293T cells, as previously detailed (Zennou et al., 2000). 6×10⁶ cells/Petri dish were cultured in DMEM and were co-transfected in a tripartite fashion with 1 ml of a mixture of: (i) 2.5 μg/ml of the pSD-GP-NDK packaging plasmid, coding for codon-optimized gag-pol-tat-rre-rev, (ii) 10 μg/ml of VSV-G Indiana envelop plasmid, and (iii) 10 μg/ml of transfer pFLAP plasmid in Hepes 1× containing 125 mM of Ca(ClO₃)₂ Supernatants were harvested at 48h post transfection, clarified by 6-minute centrifugation at 2500 rpm at 4° C., then treated for 30 min with benzonase 10 U/ml final concentration at 37° C. in Hepes-buffered solution, containing MgCl₂ (2 mM) final to eliminate residual DNA. LV vectors were aliquoted and conserved at −80° C. To determine the titers of LV preparations, HEK-293T were distributed at 4×10⁵ cell/well in flat-bottom 6-well-plates in complete DMEM in the presence of 8 μM aphidicolin (Sigma) which blocks the cell proliferation. The cells were then transduced with serial dilutions of LV preparations. The titer, proportional to the efficacy of nuclear gene transfer, is determined as Transduction Unit (TU)/ml by qPCR on total lysates at day 3 post transduction, by use of forward 5′-TGG AGG AGG AGA TAT GAG GG-3′ (SEQ ID NO: 100) and reverse 5′-CTG CTG CAC TAT ACC AGA CA-3′ (SEQ ID NO: 101) primers, specific to pFLAP plasmid and forward 5′-TCT CCT CTG ACT TCA ACA GC-3′ (SEQ ID NO: 102) and reverse 5′-CCC TGC ACT TTT TAA GAG CC-3′ (SEQ ID NO: 103) primers specific to the host housekeeping gene gadph, as described elsewhere (Iglesias et al., 2006).

1. 1.3 Mouse Studies

Female C57BL/6J mice (Janvier, Le Genest Saint Isle, France) were used between the age of 6 and 10 weeks. Male Mesocricetus auratus golden hamsters (Janvier, Le Genest Saint Isle, France) were purchased mature, i.e. 80-90 gr weight. At the beginning of the immunization regimen they weigh between 100 and 120 gr. Experimentation on animals was performed in accordance with the European and French guidelines (Directive 86/609/CEE and Decree 87-848 of 19 Oct. 1987) subsequent to approval by the Institut Pasteur Safety, Animal Care and Use Committee, protocol agreement delivered by local ethical committee (CETEA #DAP20007) and Ministry of High Education and Research APAFIS #24627-2020031117362508 v1. Mice were vaccinated with the indicated TU of LV via intraperitoneal (i.p.) injection. Sera were collected at various time points post immunization to monitor binding and neutralization activities.

1. 1.4 SARS-CoV-2 Inoculation

Ad5::hACE2-pretreated mice or hamsters were anesthetized by peritoneal injection of mixture Ketamine and Xylazine, transferred into a PSM-III where they were inoculated with 1×10⁵ TCID₅₀ of a SARS-CoV-2 clinical isolate amplified in VeroE6 cells, provided by the Centre National de Reference des Virus Respiratoires, France. The viral inoculum was contained in 20 μl for mice and in 50 μl for hamsters. Animals were then housed in an isolator in BSL3 animal facilities of Institut Pasteur. The organs and fluids recovered from the infected mice, with live SARS-CoV-2 were manipulated following the approved standard operating procedures of the BioSafety Level BSL3 facilities.

1. 1.5 Recombinant S_{CoV-2} Protein Variants

Codon-optimized nucleotide fragments encoding a stabilized foldon-trimerized version of the SARS-CoV-2 S ectodomain (a.a. 1 to 1208), the S1 monomer (a.a. 16 to 681) and the RBD subdomain (amino acid 331 to 519) both preceded by a murine IgK leader peptide, followed by an 8×His Tag (SEQ ID NO: 104) were synthetized and cloned into pcDNA™3.1/Zeom expression vector (Thermo Fisher Scientific). Proteins were produced by transient co-transfection of exponentially growing Freestyle™ 293-F suspension cells (Thermo Fisher Scientific, Waltham, Mass.) using polyethylenimine (PEI)-precipitation method as previously described (Lorin and Mouquet, 2015). Recombinant S_{CoV-2} proteins were purified by affinity chromatography using the Ni Sepharose® Excel Resin according to manufacturer's instructions (Thermo Fisher Scientific). Protein purity was evaluated by in-gel protein silver-staining using Pierce Silver Stain kit (Thermo Fisher Scientific) following SDS-PAGE in reducing and non-reducing conditions using NuPAGE™ 3-8% Tris-Acetate gels (Life Technologies). Purified proteins were dialyzed overnight against PBS using Slide-A-Lyzer® dialysis cassettes (10 kDa MW cut-off, Thermo Fisher Scientific). Protein concentration was determined using the NanoDrop™ One instrument (Thermo Fisher Scientific).

1. 1.6 ELISA

Ninety-six-well Nunc Polysorp plates (Nunc, Thermo Scientific) were coated overnight at 4° C. with 100 ng/well of purified tri-S proteins in carbonate buffer pH 9.6. After washings with PBS containing 0.1% Tween 20 (PBST), plate wells were blocked with PBS containing 1% Tween20 and 10% FBS for 2 h at room temperature. After PBST washings, 1:100-diluted sera in PBST containing 10% FBS and 4 consecutive 1:10 dilutions were added and incubated during 2h at 37° C. After PBST washings, plates were incubated with 1,000-fold diluted peroxydase-conjugated goat anti-mouse IgG/IgM (Jackson ImmunoResearch Europe Ltd, Cambridgeshire, United Kingdom) for 1 h. Plates were revealed by adding 100 μl of TMB chromogenic substrate (TMB, Eurobio Scientific) after PBST washings. Optical densities were measured at 450 nm/620 nm on a PR3100 reader following a 30 min incubation.

1. 1.7 nAb Detection

Serial dilutions of plasma were assessed for nAbs via an inhibition assay which uses Human Embryonic Kidney (HEK) 293-T cells transduced to express stably human ACE2, and safe, non-replicative S_{CoV-2} pseudo-typed LV particles which harbor the reporter luciferase firefly gene, allowing quantitation of the host cell invasion by mimicking fusion step of native SARS-CoV-2 virus (Sterlin et al.). First, 1.5×10² TU of S_{CoV-2} pseudo-typed LV were pre-incubated, during 30 min at room temperature, in U-bottom plates, with serial dilutions of each serum in a final volume of 50 μl in DMEM, completed with 10% heat-inactivated FCS and 100 U/ml penicillin and 100 μg/ml streptomycin. The samples were then transferred into clear-flat-bottom 96-well-black-plates, and each well received 2×10⁴ hACE2⁺ HEK293-T cells contained in 50 μl. After 2 days incubation at 37° C. 5% CO₂, the transduction efficiency of hACE2⁺ HEK293-T cells by pseudo-typed LV particles was determined by measuring the luciferase activity, using the Luciferase Assay System Kit with Reporter Lysis Buffer (Promega). To do so, the supernatants were completely removed from the culture wells, 40 μl of Reporter Lysis Buffer 1× and 50 μl of Luciferase Assay Reagent (Luciferase FireFly) were sequentially added to each culture well. The bioluminescent signal was quantified using an LB 960 plate reader (Berthold).

1. 1.8 SFS T-Cell Epitope Mapping

In order to map the immuno-dominant epitopes, peptides spanning the whole spike protein were pooled in ten pools, each containing 15 amino-acid residues overlapping by ten amino acids. Synthetic peptides were purchased from Mimotopes (Australia). IFN-g ELISpot assay was performed as previously described (Dion et al, 2013). These different sets of pooled peptides were used in a matrix assay to map by ICS the epitope responses induced by each construct. Peptides were dissolved in DMSO at a concentration of 2 mg/ml and diluted before use at 1 μg/ml and 2-5 μg/mL with culture medium before their use in ELISpot and ICS assays, respectively. Responses in ELISpot were considered positive if the median number of spot-forming cells in triplicate wells was at least twice that observed in control wells and at least 50 spot-forming cells per million splenocytes were detected after subtraction of the background.

1. 1.9 Generation of Ad5 Gene Transfer Vectors and Intranasal Pretreatment of Mice

The Ad5 gene transfer vectors were produced by use of ViraPower Adenoviral Promoterless Gateway Expression Kit (Thermo Fisher Scientific, France). The pCMV-BamH1-Xho1-WPRE sequence was PCR amplified from the pTRIPΔU3CMV plasmid, by use of: (i) forward primer, encoding the attB1 in the 5′ end, and (ii) reverse primer, encoding both the attB2 and SV40 polyA signal sequence in the 5′ end. The attb-PCR product was cloned into the gateway pDORN207 donor vector, via BP Clonase reaction, to form the pDORN207-CMV-BamH1-Xho1-WPRE-SV40 polyA. The hACE2 was amplified from a plasmid derivative of hACE2-expressing pcDNA3.11 (generous gift from Nicolas Escriou) while egfp was amplified from pTRIP-ieCMV-eGFP-WPRE2. The amplified PCR products were cloned into the pDORN207-CMV-BamH1-Xho1-WPRE-SV40 polyA plasmid via the BamH1 and Xho1 restriction sites. To obtain the final Ad5 plasmid, the pDORN207 vector, harboring hACE2 or gfp genes, was further inserted into pAd/PL-DEST™ vector via LR Clonase reaction.

The Ad5 virions were generated by transfecting the E3-transcomplementing HEK-293A cell line with pAd CMV-GFP-WPRE-SV40 polyA or pAd CMV-hACE2-WPRE-SV40 polyA plasmid followed by subsequent vector amplification, according to the manufacturer's protocol (ViraPower Adenoviral Promoterless Gateway Expression Kit, Thermo Fisher Scientific). The Ad5 particles were purified using Adeno-X rapid Maxi purification kit and concentrated with the Amicon Ultra-4 10k centrifugal filter unit. Vectors were resuspended and stocked à −80° C. in PIPES buffer pH 7.5, supplemented with 2.5% glucose. Ad5 were titrated using qRT-PCR protocol, as described by Gallaher et al³, adapted to HEK-293T cells.

Four days before the challenge, mice were instilled i.n. with 2.4×10⁹ IGU of Ad5::hACE2, Ad5::GFP or control empty vector resuspended in 15 μl of PBS, under general anesthesia, obtained by i.p. injection of a mixture of Ketamine (Imalgene, 100 mg/kg) and Xylazine (Rompun, 10 mg/kg).

1. 1.10 Western Blot

Expression of hACE2 in the lungs of Ad5::hACE2-transduced mice was assessed by Western Blotting. One ×10⁶ cells from lung homogenate were resolved on 4-12% NuPAGE Bis-Tris protein gels (Thermo Fisher Scientific, France), then transferred onto a nitrocellulose membrane (Biorad, France). The nitrocellulose membrane was blocked in 5% non-fat milk in 0.5% Tween PBS (PBS-T) for 2 hours at room temperature and probed overnight with goat anti-hACE2 primary Ab at 1 μg/mL (AF933, R&D systems). Following three washing intervals of 10 minutes with PBS-T, the membrane was incubated for 1 hour at room temperature with HRP-conjugated anti-goat secondary Ab and HRP-conjugated anti-β-actin (ab197277, Abcam). The membrane was washed with PBS-T thrice before visualization with enhanced chemiluminescence via the super signal west femto maximum sensitivity substrate (ThermoFisher, France) on ChemiDoc XRS+ (Biorad, France). PageRuler Plus prestained protein ladder was used as size reference.

1. 1.11 Determination of SARS-CoV-2 Viral Loads in the Lungs

Half of each lung lobes were removed aseptically and were frozen at −80° C. Organs were thawed and homogenized twice for 20 s at 4.0 m/s, using lysing matrix D (MP Biomedical) in 500 μl of ice-cold PBS. The homogenization was performed in an MP Biomedical Fastprep 24 Tissue Homogenizer. Particulate viral RNA was extracted from 70 μl of lung homogenate using QIAamp Viral RNA Mini Kit (Qiagen) according to the manufacturer's procedure. Viral load was determined following reverse transcription and real-time TaqMan® PCR essentially as described by Corman et al. (Corman et al., 2020) using SuperScript™ II Platinum One-Step Quantitative RT-PCR System (Invitrogen) and primers and probe (Eurofins) targeting SARS-CoV-2 envelope (E) gene as listed in (Table 1). In vitro transcribed RNA derived from plasmid pCI/SARS-CoV E was synthesized using T7 RiboMAX Express Large Scale RNA production system (Promega), then purified by phenol/chloroform extractions and two successive precipitations with ethanol. RNA concentration was determined by optical density measurement, then RNA was diluted to 10 genome equivalents/μL in RNAse-free water containing 100 μg/mL tRNA carrier, and stored in single-use aliquots at −80° C. Serial dilutions of this in vitro transcribed RNA were prepared in RNAse-free water containing 10 μg/ml tRNA carrier and used to establish a standard curve in each assay. Thermal cycling conditions were: (i) reverse transcription at 55° C. for 10 min, (ii) enzyme inactivation at 95° C. for 3 min, and (iii) 45 cycles of denaturation/amplification at 95° C. for 15 s, 58° C. for 30 s. Products were analyzed on an ABI 7500 Fast real-time PCR system (Applied Biosystems).

1. 1.12 Cytometric Analysis of Lung Innate Immune Cells

Lungs from individual mice were treated with collagenase-DNAse-I for 30-minute incubation at 370 C and homogenized by use of GentleMacs. Cells were and filtered through 100 μm-pore filters and centrifuged at 1200 rpm during 8 minutes. Cells were then treated with Red Blood Cell Lysing Buffer (Sigma), washed twice in PBS. Cells were then stained as following. (i) To detect DC, monocytes, alveolar and interstitial macrophages: Near IR Live/Dead (Invitrogen), FcγII/III receptor blocking anti-CD16/CD32 (BD Biosciences), BV605-anti-CD45 (BD Biosciences), PE-anti-CD11b (eBioscience), PE-Cy7-antiCD11c (eBioscience), BV450-anti-CD64 (BD Biosciences), FITC-anti-CD24 (BD Biosciences), BV711-anti-CD103 (BioLegend), AF700-anti-MHC-II (BioLegend), PerCP-Cy5.5-anti-Ly6C (eBioscience) and APC anti-Ly-6G (Miltenyi) mAbs, (ii) to detect neutrophils or eosinophils: Near IR DL (Invitrogen), FcγII/III receptor blocking anti-CD16/CD32 (BD Biosciences), PerCP-Vio700-anti-CD45 (Miltenyi), APC-anti-CD11 b (BD Biosciences), PE-Cy7-anti-CD11c (eBioscience), FITC-anti-CD24 (BD Biosciences), AF700-anti-MHC-II (BioLegend), PE-anti-Ly6G (BioLegend), BV421-anti-Siglec-F (BD Biosciences), (iii) to detect mast cells, basophils, NK: Near IR DL (Invitrogen), BV605-anti-CD45 (BD Biosciences), PE-anti-CD11b (eBioscience), eF450-anti-CD11c (eBioscience), PE-Cy7-anti-CD117 (BD Biosciences), APC-anti-FcER1 (BioLegend), AF700-anti-NKp46 (BD Biosciences), FITC-anti-CCR3 (BioLegend), without FcγII/III receptor blocking anti-CD16/CD32. Cells were incubated with appropriate mixtures for 25 minutes at 4° C. Cells were then washed twice in PBS containing 3% FCS and then fixed PFA 4% and overnight incubation at 4° C. The cells were acquired in an Attune NxT cytometer system (Invitrogen) and data were analyzed by FlowJo software (Treestar, OR, USA).

1.1.13 qRT-PCR Detection of Inflammatory Cytokines and Chemokines in the Lungs

Lung samples were added to lysing matrix D (MP Biomedical) containing 1 mL of TRIzol reagent and homogenized during 30 seconds at 6.0 m/s, twice using MP Biomedical Fastprep 24 Tissue Homogenizer. Total RNA was extracted using TRIzol reagent (ThermoFisher Scientific, France), according to the manufacturer's procedure. cDNA was synthesized from 4 μg of RNA in the presence of 2.5 μM of oligo(dT) 18 primers (SEQ ID NO: 105), 0.5 mM of deoxyribonucleotides, 2.0 U of RNase Inhibitor and SuperScript IV Reverse Transcriptase (ThermoFisher Scientific, France) in 20 μl reaction. The real-time PCR was performed on QuantStudio™ 7 Flex Real-Time PCR System (ThermoFisher Scientific, France). Reactions were performed in triplicates in a final reaction volume of 10 μl containing 5 μl of iQ™ SYBR® Green Supermix (Biorad, France), 4 μl of cDNA diluted 1:15 in DEPC-water and 0.5 μl of each forward and reverse primers at a final concentration of 0.5 μM (Table 2). The following thermal profile was used: a single cycle of polymerase activation for 3 min at 95° C., followed by 40 amplification cycles of 15 sec at 95° C. and 30 sec 60° C. (annealing-extension step). The average CT values were calculated from the technical replicates for relative quantification of target cytokines/chemokines. The differences in the CT cytokines/chemokines amplicons and the CT of the reference β-globin, termed ACT, were calculated to normalized for differences in the quantity of nucleic acid. The ACT of experimental condition were compared relatively to the PBS-treated mice using the comparative ΔΔCT method. The fold change in gene expression was further calculated using 2-ΔΔCT.

### Example 1.2: Induction of Antibody Responses by LV Coding SARS-CoV-2 Spike Protein Variants

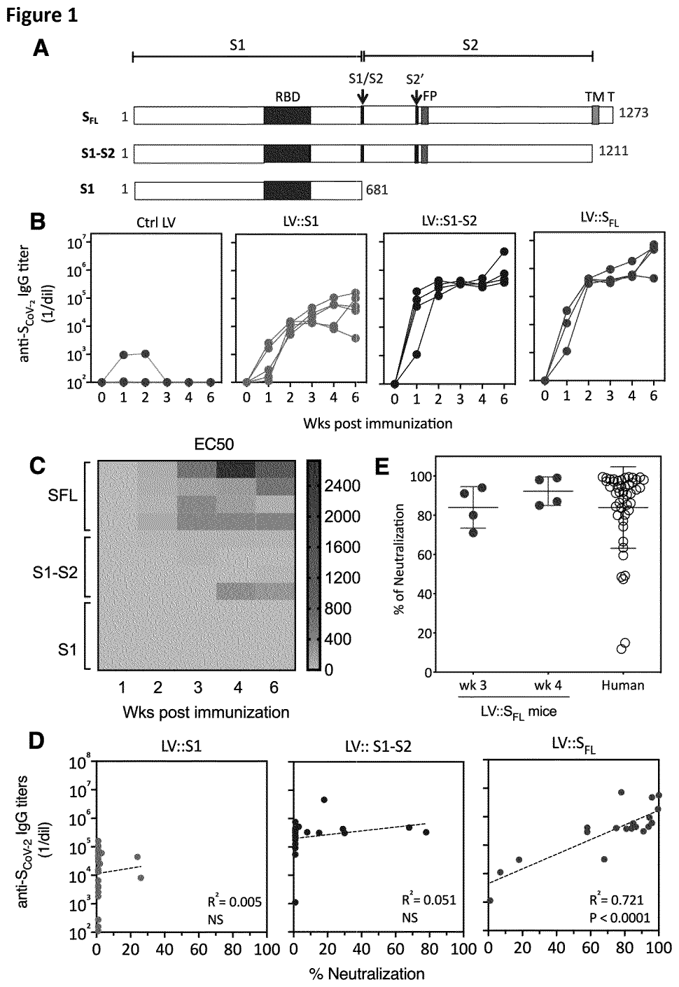

To develop a vaccine candidate able to induce nAbs specific to S_{CoV-2}, we generated LV encoding, under the transcriptional control of the cytomegalovirus (CMV) immediate-early promoter, for codon-optimized sequences of: (i) full-length, membrane anchored form of S (LV::S_{FL}), (ii) S1-S2 ecto-domain, without the transmembrane and C-terminal short internal tail (LV::S1-S2), or (iii) S1 alone (LV::S1), which all harbor the RBD (FIG. 1A), with prospective conformational heterogeneities. To evaluate the humoral responses induced by these vectors, C57BL/6 mice (n=4/group) were immunized by a single i.p. injection of 1×10⁷ TU/mouse of either LV, or an LV encoding GFP as negative control. S_{CoV-2}-specific Ab responses were investigated in the sera at weeks 1, 2, 3, 4 and 6 post immunization. In LV::S_{FL} or LV::S1-S2-immunized mice, S_{CoV-2}-specific immunoglobulin G (IgG) were detectable as early as 1 week post immunization and their amounts exhibited a progressive increment until week 6 post immunization with Mean titer±SEM of (4.5±2.9)×106 or (1.5±1)×10⁶, respectively. In comparison, S_{CoV-2}-specific IgG titers were 100× lower, i.e., (7.1±6.1)×10⁴, in their LV::S1-immunized counterparts (FIG. 1B).

Sera were then evaluated for their capacity to neutralize SARS-CoV-2, using a reliable neutralization assay based on nAb-mediated inhibition of hACE2⁺ cell invasion by non-replicative LV particle surrogates, pseudo-typed with S_{CoV-2} (Sterlin et al.). Such S_{CoV-2} pseudo-typed LV particles, harbor the reporter luciferase gene, which allows quantitation of the hACE2⁺ host cell invasion, inversely proportional to the neutralization efficiency of nAbs possibly contained in the biological fluids. Analysis of 50% Effective Concentrations (EC50) of the sera from the LV::S_{FL}-, LV::S1-S2- or LV::S1-immunized mice clearly established that LV::S_{FL} was the most potent vector at inducing S_{CoV-2}-specific nAbs (FIG. 1C). Moreover, nAb titers were correlated with S_{CoV-2}-specific IgG titers only in the sera of LV::S_{FL}-immunized mice (p<0.0001, R2=0.645, two-sided Spearman rank-correlation test) (FIG. 1E). These results strongly suggest that in the S1-S2 or S1 polypeptides, the conformations of the pertinent B-cell epitopes are distinct from those of the native SFS, the latter representing the only variant which induces nAbs able to inhibit the S_{CoV-2}-hACE2 interaction and host cell invasion. Comparison of the neutralizing capacity of sera from the LV::S_{FL}-immunized mice and a cohort of mildly symptomatic infected people living in Crépy en Valois, one of the first epidemic zones appeared in France, showed equivalent neutralizing activity average (FIG. 1D). These data predicted a protective potential of the humoral response induced by LV::S_{FL}.

In order to potentially increase the immunogenicity of LV::S vectors at inducing neutralizing Abs, we generated LV vectors coding for stabilized pre-fusion S_{CoV-2}, engineered as follows:

(i) S_{CoV-2} with prospective increased stability, harboring two 986K→P and 987V→P consecutive a.a. substitution. It is indeed established that the a.a substitution toward the rigid proline residue increases the protein stability by decreasing the conformational entropy.

(ii) S_{CoV-2} with the 681 PRRARS686 (SEQ ID NO: 22)→681PGSAGS686 (SEQ ID NO: 23) a.a. substitution at the furin cleavage site, thereby unrecognizable by this proteolytic enzyme.

(iii) S_{CoV-2} harboring the 986K→P and 987V→P consecutive a.a. substitutions, and deleted for the 675 ^{QTQTNSPRRAR} 685 (SEQ ID NO: 24), encompassing the furin cleavage site.

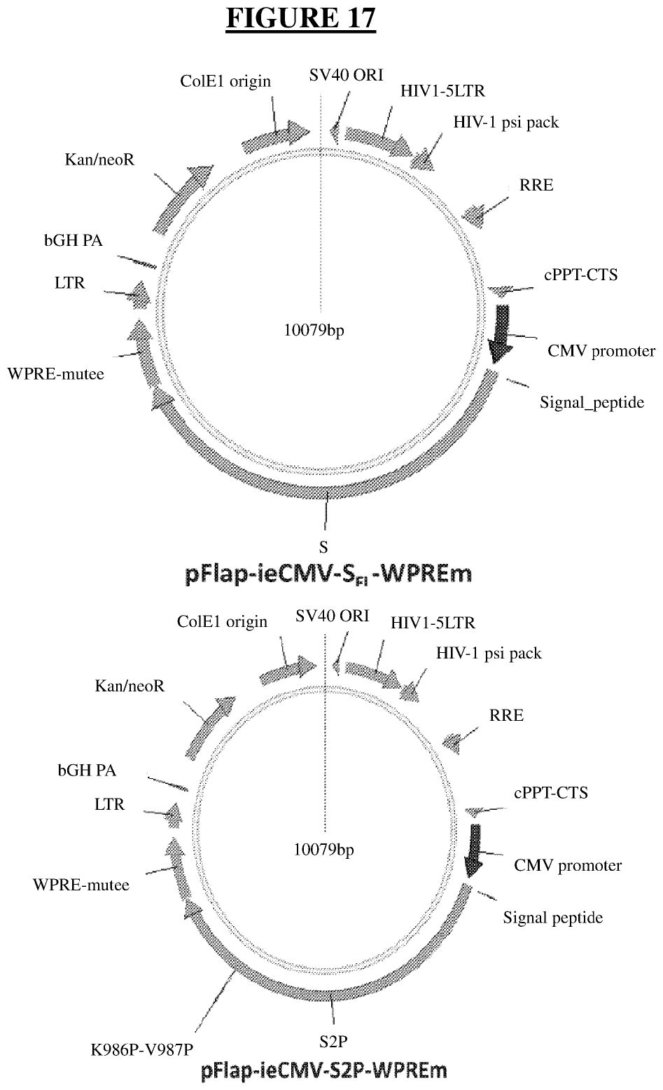

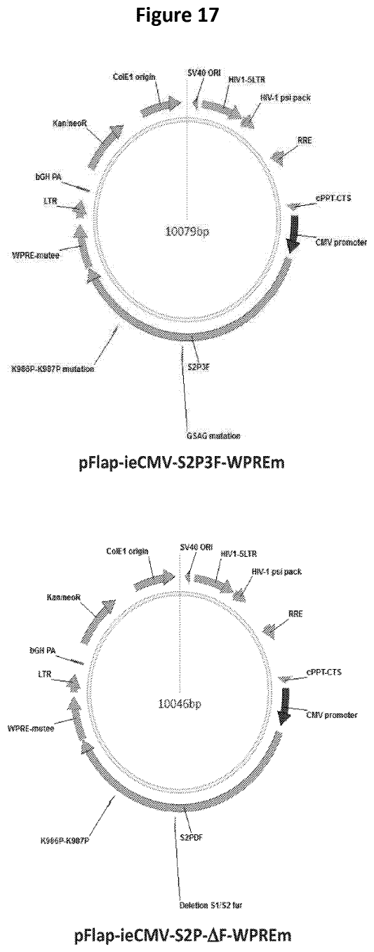

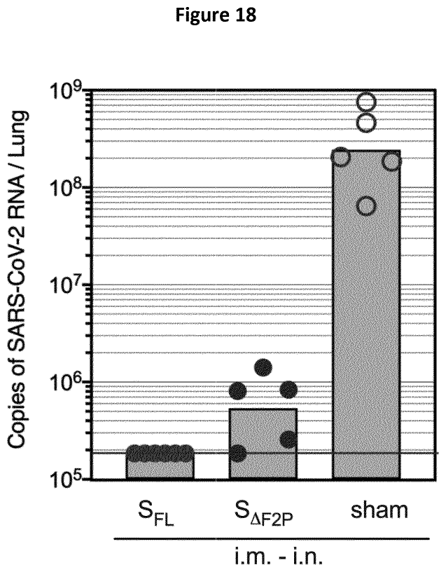

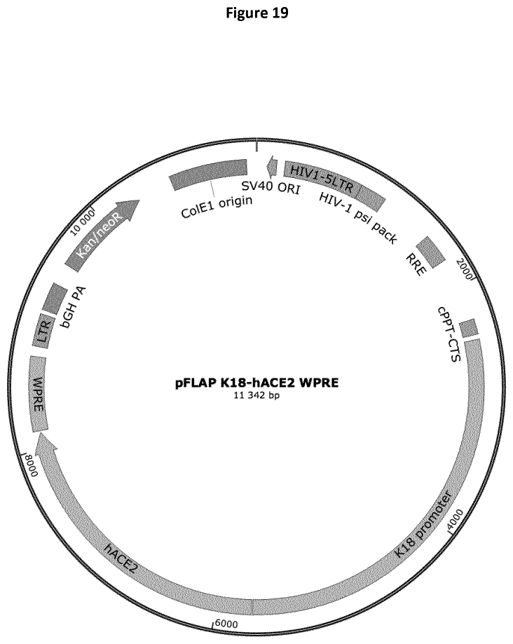

FIG. 17A shows the plasmid map of pFlap-ieCMV-SFS-WPREm.

The nucleotide sequence of pFlap-ieCMV-SFL-WPREm is shown in FIG. 20A where it is identified as SEQ ID NO: 3. The nucleotide sequence encoding the S protein present in this vector is shown in FIG. 20B where it is identified as SEQ ID NO: 4. The amino acid sequence encoding the S protein present in this vector is shown in FIG. 20C where it is identified as SEQ ID NO: 5.

FIG. 17B shows the plasmid map of pFlap-ieCMV-S2P-WPREm.

The nucleotide sequence of pFlap-ieCMV-S2P-WPREm is shown in FIG. 21A where it is identified as SEQ ID NO: 6. The nucleotide sequence encoding the S protein present in this vector is shown in FIG. 21B where it is identified as SEQ ID NO: 7. The amino acid sequence encoding the S protein present in this vector is shown in FIG. 21C where it is identified as SEQ ID NO: 8.

FIG. 17C shows the plasmid map of pFlap-ieCMV-S2P3F-WPREm.

The nucleotide sequence of pFlap-ieCMV-S2P3F-WPREm is shown in FIG. 22A where it is identified as SEQ ID NO: 9. The nucleotide sequence encoding the S protein present in this vector is shown in FIG. 22B where it is identified as SEQ ID NO: 10. The amino acid sequence encoding the S protein present in this vector is shown in FIG. 22C where it is identified as SEQ ID NO: 11.

FIG. 17D shows the plasmid map of pFlap-ieCMV-S2PdeltaF-WPREm.

The nucleotide sequence of pFlap-ieCMV-S2PdeltaF-WPREm is shown in FIG. 23A where it is identified as SEQ ID NO: 12. The nucleotide sequence encoding the S protein present in this vector is shown in FIG. 23B where it is identified as SEQ ID NO: 13. The amino acid sequence encoding the S protein present in this vector is shown in FIG. 23C where it is identified as SEQ ID NO: 14.

The COLLECTION NATIONALE DE CULTURES DE MICROORGANISMS (CNCM) has the status of International Depositary Authority under the Budapest Treaty on the International Recognition of the Deposit of Microorganisms for the Purposes of Patent Procedure. The CNCM is located at Institut Pasteur, 25-28 rue du Docteur Roux, 75724 Paris Cedex 15 FRANCE.

The following materials were deposited on Jul. 15, 2020: pFlap-ieCMV-S2PdeltaF-WPREm (CNCM I-5537), pFlap-ieCMV-S2P3F-WPREm (CNCM I-5538), pFlap-ieCMV-S2P-WPREm (CNCM I-5539), and pFlap-ieCMV-SFL-WPREm (CNCM I-5540). Deposit receipts are filed herewith.

The following materials were deposited on Jul. 6, 2021 at the CNCM: pFlap-ieCMV-S-B1.1.7-WPREm (CNCM I-5708), pFlap-ieCMV-S-B351-WPREm (CNCM I-5709), pFlap-ieCMV-S-B351-2P-WPREm (CNCM I-5710), pFlap-ieCMV-SFL-D614G-WPREm (CNCM I-5711), pFlap-ieCMV-S-P1-WPREm (CNCM I-5712). Deposit receipts are filed herewith.

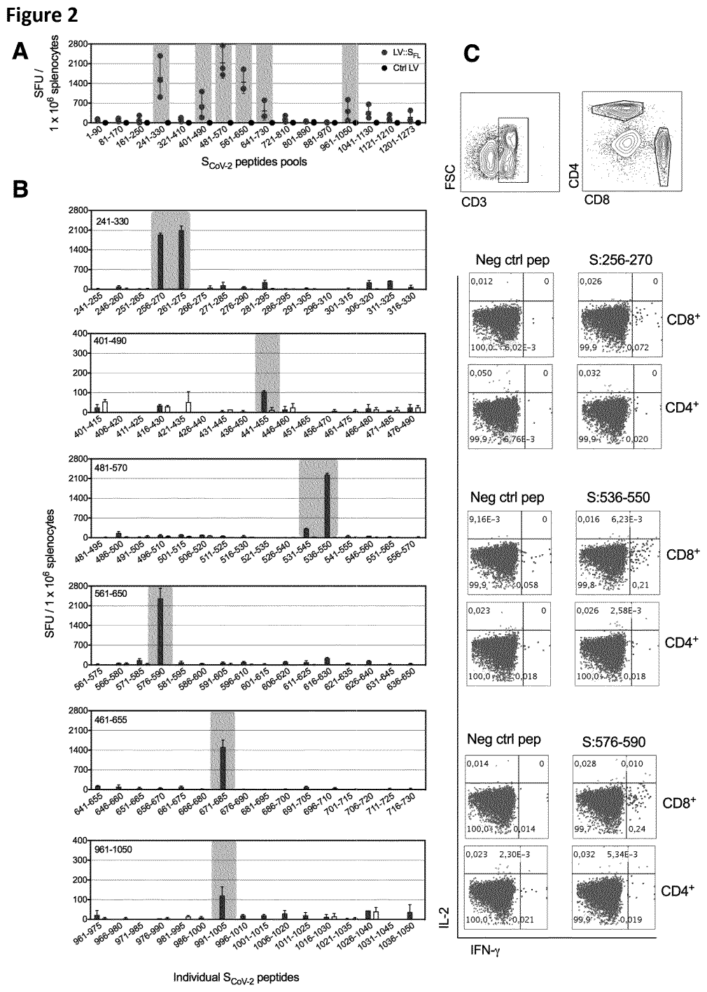

LV::S_{FL}-immunized C57BL/6 mice (n=3) also displayed strong anti-S_{CoV-2} T-cell responses, as detected at week 2 post immunization by IFNγ ELISPOT-based epitope mapping, applied to splenocytes stimulated with distinct pools of 15-mer peptides spanning the full-length S_{CoV-2} (FIG. 2A). Significant amounts of responding T cells were detected for 6 out of 16 peptide pools. Deconvolution of these positive pools allowed identification of S:256-275 (SGWTAGAAAYYVGYLQPRTF—SEQ ID No.32), S:536-550 (NKCVNFNFNGLTGTG—SEQ ID No.16) and S:576:590 (VRDPQTLEILDITPC—SEQ ID No.17) immunodominant epitopes, giving rise to >2000 Spot Forming Unit (SFU)/1×10⁶ splenocytes (FIG. 2B). These epitopes elicited CD8⁺—but not CD4⁺—T cells, as assessed by intracellular cytokine staining (FIG. 2C). The predominant CD8⁺ phenotype of these T cells is in accordance with the favored orientation of LV-encoded antigens to the MHC-I presentation pathway (Hu et al., 2011). We also identified S:441-455 (LDSKVGGNYNYLYRL—SEQ ID No.18), S:671-685 (CASYQTQTNSPRRAR SEQ ID No.19) and S:991-1005 (VQIDRLITGRLQSLQ—SEQ ID No.20) subdominant epitopes, which gave rise to <2000 SFU/1×10⁶ splenocytes in ELISPOT assay (FIG. 2B).

### Example 1.3: Set Up of a Murine Model Expressing Human ACE2 in the Respiratory Tracts, Using an Ad5 Gene Delivery Vector

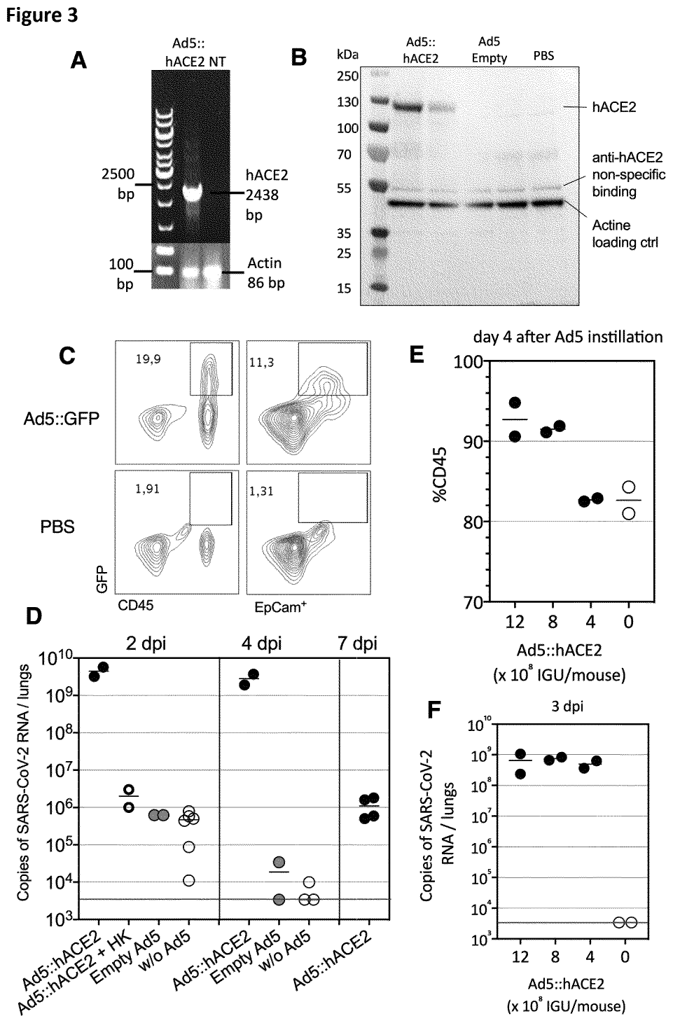

As S_{CoV-2} does not interact efficaciously with murine ACE2, wild-type laboratory mice are not permissive to replication of SARS-CoV-2 clinical isolates. Due to unavailability of hACE2 transgenic mice in Europe during the progression of the present study, to evaluate the LV::S_{FL} vaccine efficacy, we sought to elaborate a murine model in which the hACE2 expression is induced in the respiratory tracts and pulmonary mucosa. To do so, we generated an Ad5 gene delivery vector able to vehicle in non-integrating episomes, the gene coding for hACE2 under the transcriptional control of CMV promoter (Ad5::hACE2). We first checked in vitro the potential of the Ad5::hACE2 vector to transduce HEK293T cells by RT-PCR (FIG. 3A). To achieve in vivo transduction of respiratory tract cells, we instilled i.n. 2.5×10⁹ IGU/mouse of Ad5::hACE2 into C57BL/6 mice. Four days later, the hACE2 protein expression was detectable in the lung cell homogenate by Western Blot (FIG. 3B). To get more insights into the in vivo expression profile of a transgene administered under these conditions, we instilled i.n. the same dose of an Ad5::GFP reporter vector into C57BL/6 mice. As evaluated by cytometry, 4 days post instillation, the GFP reporter was expressed not only in the lung epithelial EpCam⁺ cells, but also in lung immune cells, as tracked by CD45 pan-hematopoietic marker (FIG. 3C), showing that this approach allows efficient transduction of epithelial cells, which however is not restricted to these cells.

To evaluate the permissibility of such hACE2-transduced mice to SARS-CoV-2 infection, 4 days after i.n. pretreatment with either Ad5::hACE2 or an empty control Ad5 vector, C57BL/6 mice were inoculated i.n. with 1×10⁵ TCID₅₀ of a SARS-CoV-2 clinical isolate, which was isolated in February 2020 from a COVID-19 patient by the National Reference Centre for Respiratory Viruses (Institut Pasteur, France). The lung viral loads, determined at 2 days post inoculation (dpi), were as high as (4.4±1.8)×109 copies of SARS-CoV-2 RNA/mouse in Ad5::hACE2-pretreated mice, compared to only (6.2±0.5)×10⁵ copies/mouse in empty Ad5-pretreated, or (4.0±2.9)×105 copies/mouse in un-pretreated mice (FIG. 3D). At 4 dpi, the lung viral loads were maintained in Ad5::hACE2-pretreated mice (2.8±1.3×10⁹ copies/mouse), whereas a drop to (1.7±2.3)×104 or (3.9±5.1)×103 copies/mouse was observed in empty Ad5-pretreated or unpretreated mice, respectively. At 7 dpi, in Ad5::hACE2-pretreated mice, the viral loads decreased significantly, albeit were still largely detectable ((1.33±0.9)×106 copies/mouse).

Ad5::hACE-2 i.n. instillation induced CD45⁺ cell recruitment to the lungs, however, this effect was reduced with decreasing vector doses, as determined at day 4 post instillation. The dose of 4×10⁸ IGU/mouse did not cause CD45⁺ cell recruitment, as compared to the PBS-treated controls (FIG. 3E), while still conferred full permissibility to SARS-CoV-2 replication (FIG. 3F). The permissibility of Ad5-hACE2-pretreated mice to SARS-CoV-2 replication and the set-up of this model paved the way for the in vivo assessment of vaccine or drug efficacy against SARS-CoV-2 in mice.

### Example 1.4: Evaluation of the Protective Potential of LV::SFL Against SARS-CoV-2 in Mice

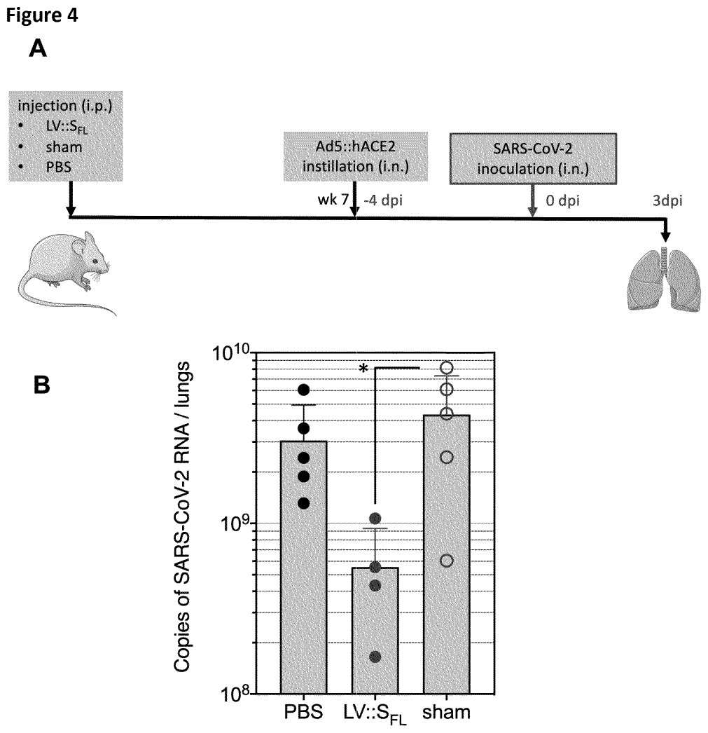

To investigate the prophylactic potential of LV::S_{FL} against SARS-CoV-2, C57BL/6 mice (n=4/group) were injected i.p. with a single dose of 1×10⁷ TU/mouse of LV::S_{FL} or a negative control LV (sham). At week 6 post immunization, the mice were pretreated with Ad5::hACE2, and 4 days later, they were inoculated i.n. with 1×10⁵ TCID₅₀ of SARS-CoV-2 (FIG. 4A). At 3 dpi, the lung viral loads in LV::S_{FL}-vaccinated mice was reduced by ˜1 log₁₀, i.e., Mean±SEM of (3.2±2.2)×10⁸ SARS-CoV-2 RNA copies/mouse, respectively compared to (1.7±0.9)×10⁹ or (2.4±1.6)×10⁹ copies/mouse in the un- or sham-vaccinated mice (FIG. 4B). Therefore, a single LV::S_{FL} injection effectively afforded ˜90% inhibition of the viral replication in the lungs.

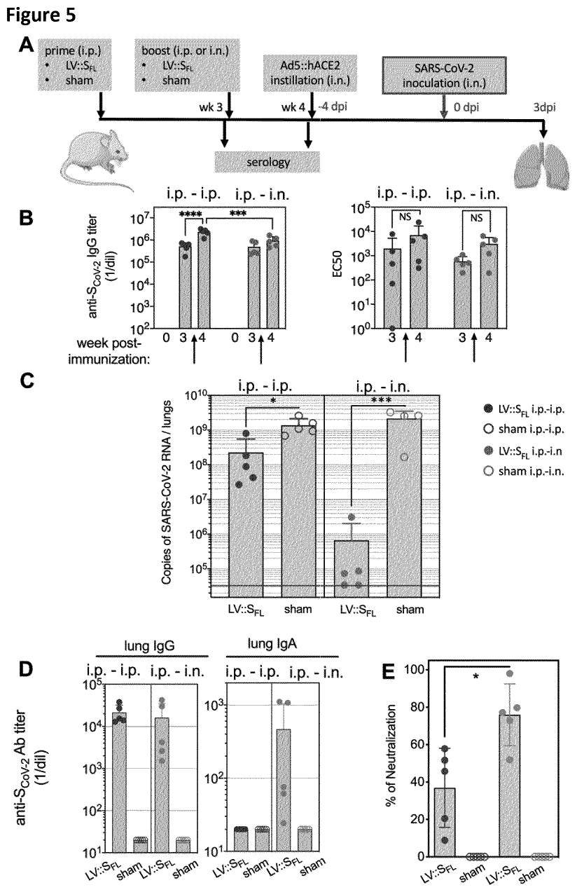

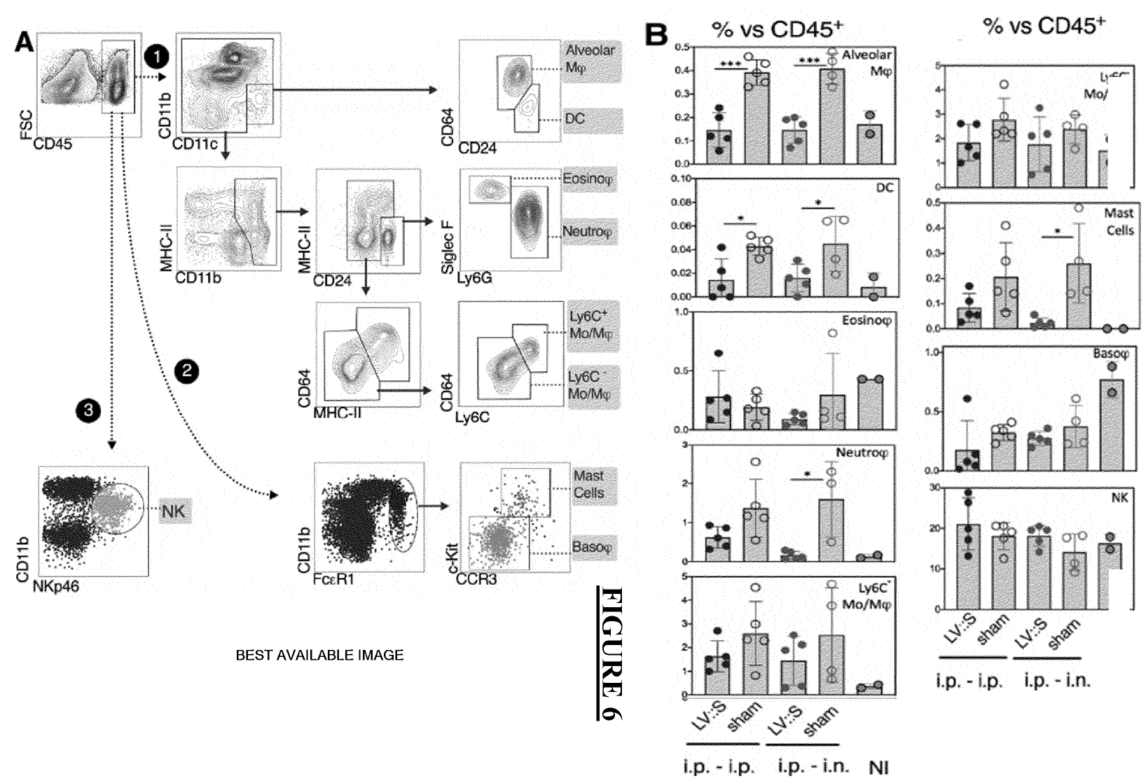

To further improve the prophylactic effect, we evaluated the prime-boost or prime-target approaches. C57BL/6 mice (n=4-5/group) were primed i.p. with 1×10⁷ TU of LV::S_{FL} or a control LV at week 0, and then boosted at week 3 with: (i) 1×10⁷ TU of the same LV via the i.p. route (“LV::S_{FL} i.p.-i.p.”, prime-boost), or (ii) with 3×10⁷ TU via the i.n. route (“LV::S_{FL} i.p.-i.n.”, prime-target) to attract the mediators of systemic immunity to the lung mucosa (FIG. 5A). Systemic boosting with LV::S_{FL} via i.p. resulted in a significant increase in the anti-S_{CoV-2} IgG titers (FIG. 5B, left). In contrast, mucosal targeting with LV::S_{FL} via i.n. did not lead to a statistically significant improvement of anti-S_{CoV-2} IgG titers at the systemic level (FIG. 5B left). In terms of serum neutralization potential, even though a trend to increase was observed after i.p. or i.n. boost, the differences did not reach statistical significance (FIG. 5B right).

All mice were then pretreated with Ad5::hACE2 and challenged i.n. with 0.3×10⁵ TCID₅₀ of SARS-CoV-2 at week 4 post prime. At 3 dpi, the lung viral loads were significantly lower in LV::S_{FL} i.p.-i.p. immunized mice, i.e., mean±SD (2.3±3.2)×10⁸, than in sham-vaccinated mice (13.7±7.5)×10⁸ copies of SARS-CoV-2 RNA, (FIG. 5C) This viral load reduction was similar to that obtained with a single LV::S_{FL} administration (FIG. 5C). Most importantly, after i.n. LV::S_{FL} target immunization, >3 log 10 decrease in viral loads was observed and 2 out of 5 mice harbored undetectable lung viral loads as determined by qRT-PCR assay. Anti-S_{CoV-2} IgG were in fact detected in the clarified lung homogenates of the partially (LV::S_{FL} i.p.-i.p.) or the fully (LV::S_{FL} i.p.-i.n.) protected mice. In contrast anti-S_{CoV-2} IgA were only detectable in the fully protected LV::S_{FL} i.p.-i.n. mice (FIG. 5D). Higher neutralizing activity was detected in the clarified lung homogenates of LV::S_{FL} i.p.-i.n. mice than of their LV::S_{FL} i.p.-i.p. counterparts (FIG. 5E). Therefore, increasing the titers of NAb of IgG isotype at the systemic levels did not improve the protection against SARS-CoV-2. However, a mucosal i.n. target immunization, with the potential to attract immune effectors to the entry point of the virus to the host organism and able to induce local IgA Abs, correlated with the inhibition of SARS-CoV-2 replication.

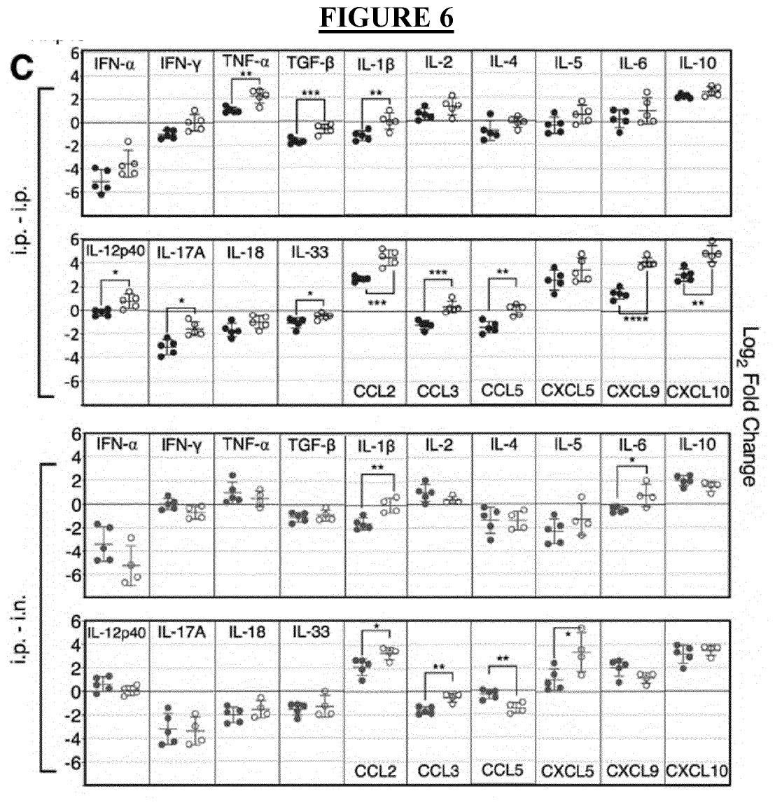

Based on the compelling evidences of innate immune hyperactivity in the acute lung injury in COVID-19 (Vabret et al., 2020), we investigated the possible variations of the lung innate immune cell subsets (FIG. 6A), in the non-infected controls, sham-vaccinated or LV::S_{FL}-vaccinated mice inoculated with SARS-CoV-2. At 3 dpi, we detected no differences in the proportions of basophils or NK cells versus total lung CD45⁺ cells, among various experimental groups (FIG. 6B). In net contrast, we detected increased proportions of alveolar macrophages, dendritic cells, mast cells, eosinophils, Ly6C⁺ or Ly6C⁻ monocytes/macrophages or neutrophils versus total lung CD45⁺ cells, in sham-vaccinated mice which displayed the highest lung viral loads. These observations demonstrate that in this mouse model, the increased lung SARS-CoV-2 loads are correlated with recruitment of several inflammation-related innate immune cells, and that vaccine-mediated anti-viral protection dampens or avoids such inflammation. This was corroborated with the reduced cytokine and chemokine contents in the lungs of mice vaccinated by prime-boost/target with LV::S_{FL}, as evaluated by qRT-PCR applied to RNA extracted from the total lung homogenates (FIG. 6C). Therefore, the conferred protection also avoided pulmonary inflammation linked to SARS-CoV-2 infection.

### Example 1.5: Evaluation of the Protective Potential of LV::S_{FL} Against SARS-CoV-2 in Golden Hamsters

Outbred Mesocricetus auratus, so-called golden hamsters, provide a suitable pre-clinical model to study the COVID-19 pathology, as the ACE2 ortholog of this species interacts efficaciously with S_{CoV-2}, whereby host cell invasion and viral replication (Sia et al., 2020). We thus investigated the prophylactic effect of LV::S_{FL} vaccination on SARS-CoV-2 infection in this pertinent model. Although integrative LV vectors are largely safe and passed successfully a phase 1 clinical trial (2011-006260-52 EN), in addition to the integrative LV::S_{FL}, we also evaluated an integrase deficient, non-integrative version of LV::S_{FL} with the prospect of application un future clinical trials.

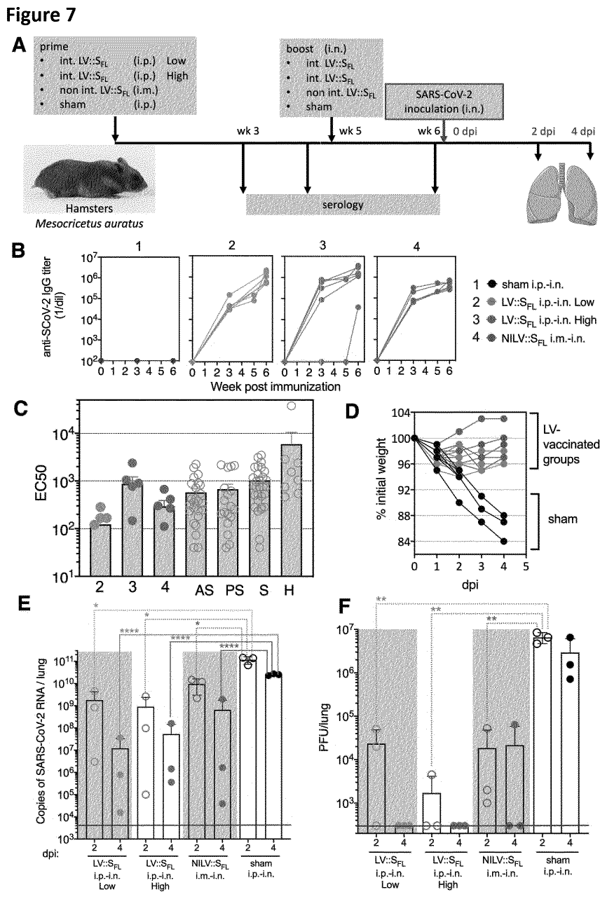

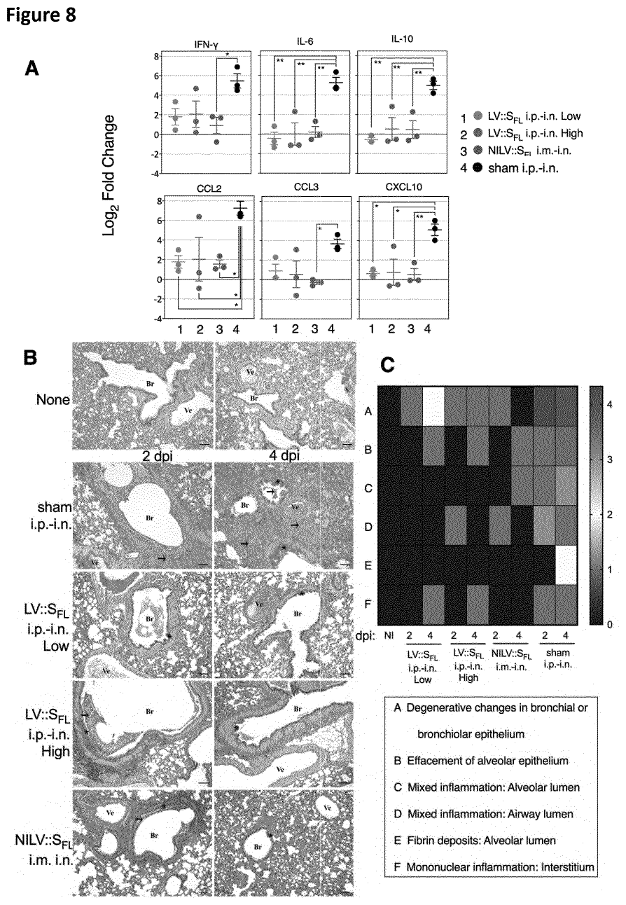

To assess the prophylactic effect of vaccination following prime-boost/target regimen, M. auratus hamsters (n=6/group) were: (i) primed i.p. with the low dose of 1×10⁶ TU of integrative LV::S_{FL} and boosted i.n. at week 4 with 3×10⁷ TU of integrative LV::S_{FL}, (“int LV::S_{FL} i.p.-i.n. Low”), (ii) primed i.p. with the high dose of 1×10⁷ TU of integrative LV::S_{FL} and boosted i.n. at week 4 with 3×10⁷ TU of integrative LV::S_{FL} (“int LV::S_{FL} i.p.-i.n. High”), or (iii) primed intramuscularly (i.m.) with 1×10⁸ TU of non-integrative LV::S_{FL} and boosted i.n. at week 4 with 3×10⁷ TU of non-integrative LV::S_{FL} (“non int LV::S_{FL} i.m.-i.n.”) (FIG. 7A). Sham-vaccinated controls received the same amounts of an empty integrative LV via i.p. and i.n. routes. Comparable S_{CoV-2}-specific IgG antibodies were detected by ELISA in the sera of hamsters from various vaccinated groups, before and after the i.n. boost (FIG. 7B). Post boost/target serology detected neutralization activity in all the groups, with the highest EC50 average observed in “int LV::S_{FL} i.p.-i.n. High” group. Such levels were comparable to those detected in asymptomatic, pauci-symptomatic, symptomatic or healthy COVID-19 contacts in humans (FIG. 7C). All the hamsters were challenged i.n. with 0.3×10⁵ TCID₅₀ of SARS-CoV-2 at week 5. Up to 16% weight loss was progressively reached at 4 dpi in sham-vaccinated individuals, compared to non-significant loss in all the LV::S_{FL}-vaccinated groups (FIG. 7D). At 2 dpi, decreases of ˜1.5-to-3 log₁₀ were observed in the lung viral loads of “int LV::S_{FL} i.p.-i.n. Low”, “int LV::S_{FL} i.p.-i.n. High” and “non int LV::S_{FL} i.m.-i.n.” groups, compared to sham-vaccinated hamsters (FIG. 7E, F). At 4 dpi, the magnitude of viral load reductions in the vaccinated groups were still higher and reached >4 log₁₀, compared to the sham-vaccinated individuals. More immunological and histopathological studies confirmed the substantial lung protection by LV vaccination in the hamster model. (FIG. 8).

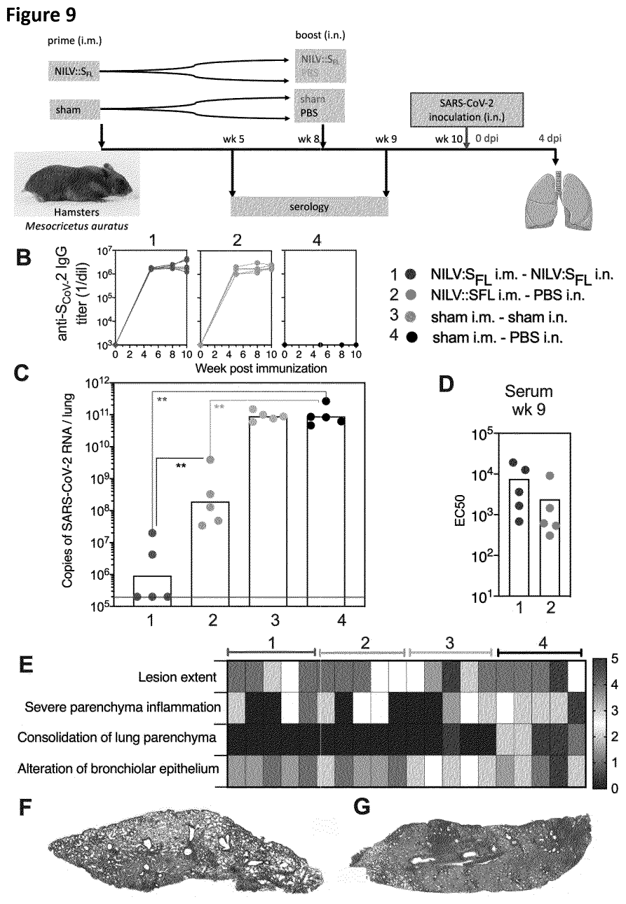

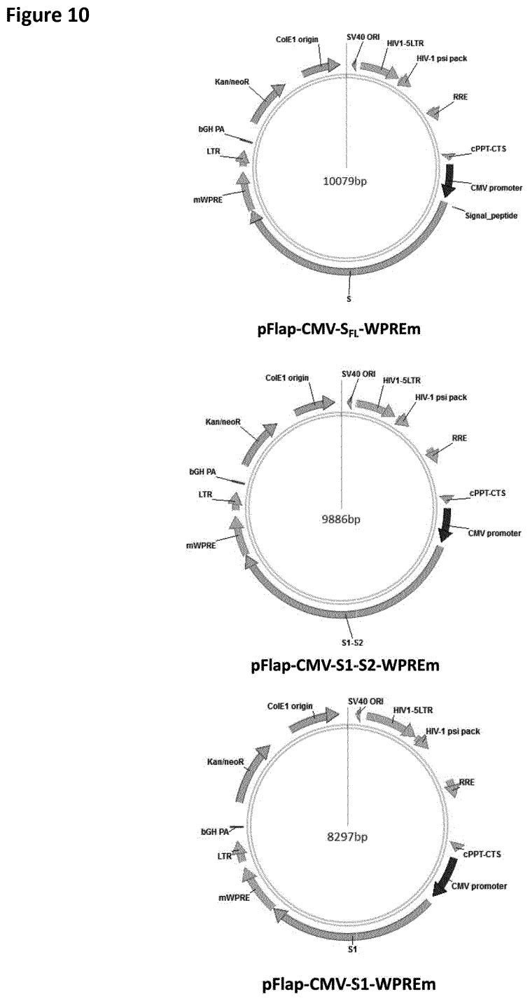

In an additional experiment (FIG. 9A), we showed that: (i) a single i.m. injection of NILV::S_{FL} induced high titers of serum anti-S Abs (FIG. 9B), and initiated significant—but partial—levels of protection in the lungs (FIG. 9C), and, (ii) an i.n. boost with NILV::S_{FL} which did not improve the serum NAb activity (FIG. 9D), induced significantly improved protection against SARS-CoV-2, as determined by the lung viral loads, based on qRT-PCR (FIG. 9C), detected at 4 dpi. At 4 dpi, in sham-vaccinated and challenged hamsters, substantial pulmonary lesions, severe parenchyma inflammation, consolidation of pulmonary parenchyma, marked alteration of bronchiolar epithelium and moderate effacement of the bronchiolar epithelium were detected (FIG. 9E). In their NILV::S_{FL}-vaccinated counterparts, boosted or not, pulmonary lesions were clearly of lower severity (FIG. 9E, F, G).

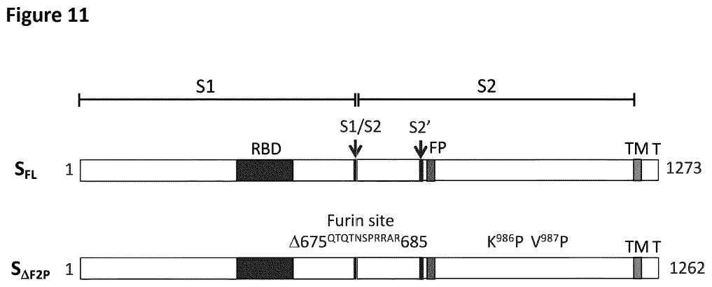

Sterilizing protection in hamster model by a single i.n. NILV::S_{ΔF2P} administration We generated LV encoding a prefusion form of S_{CoV-2} under transcriptional control of the cytomegalovirus promoter. This prefusion S_{CoV-2} variant (S_{ΔF2P}) has a deletion of 675^{QTQTNSPRRAR}685 (SEQ ID NO: 24) sequence, encompassing the polybasic RRAR (SEQ ID NO: 99) furin cleavage site, at the boundary of S1/S2 subunits, and harbors K⁹⁸⁶P and V⁹⁸⁷P consecutive proline substitutions in S2, within the hinge loop between heptad repeat 1 and the central helix (FIG. 11).

We also assessed the prophylactic effect of vaccination with only a single i.n. administration of NILV::S_{ΔF2P} in the hamster model.

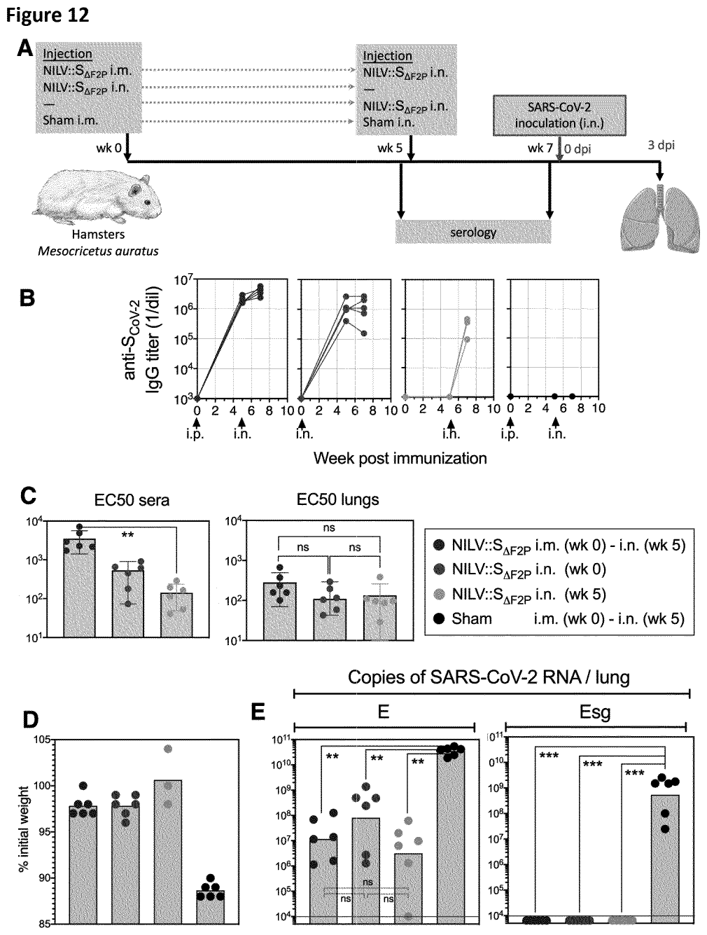

Hamsters (n=6/group) were: (i) primed i.m. at wk 0 with 1×10⁸ TU of NILV::S_{ΔF2P} and boosted i.n. at wk 5 with the same amount of the vector, as a positive protection control, (ii) immunized i.n. with a single injection of 1×10⁸ TU of NILV::S_{ΔF2P} at wk 0, or (iii) at wk 5 (FIG. 12A). Sham-vaccinated controls received equivalent amounts of an empty NILV via i.n. at wks 0 and 5. Comparable and high titers of anti-S_{CoV-2} IgG Abs were detected in the sera in the first two groups at wk 5 (FIG. 12B). At wk 7, the serum Ab titer was maintained high in the NILV::S_{ΔF2P} i.m.-i.n. group while it was slightly decreased in some individuals of the “NILV::S_{ΔF2P} i.n. wk 0” group. At this time point, in the “NILV::S_{ΔF2P} i.n. wk 5” group, lower serum Ab titers were detected (FIG. 12B). Although the virus neutralization activity was significantly lower in the sera of “NILV::S_{ΔF2P} i.n. wk 5” hamsters compared to the two other vaccinated groups, these individuals had an equivalent neutralizing capacity in their lung homogenates (FIG. 12C).

At wk 7, all animals were challenged i.n. with 0.3×10⁵ TCID₅₀ of a SARS-CoV-2. At 4 days post inoculation (dpi), only 2-3% weight loss was detected in the NILV::S_{ΔF2P}-vaccinated groups, compared to 12% in sham-vaccinated hamsters (FIG. 12D). At this time point, as determined by qRT-PCR detecting SARS-CoV-2 Envelop (E_{CoV-2}) RNA, 2-to-3 log 10 decreases were observed in NILV::S_{ΔF2P}-vaccinated individuals of either i.m.-i.n. or single i.n. groups, compared to sham-vaccinated group (FIG. 12E). Assessment of lung viral loads by a qRT-PCR which detects sub-genomic E_{CoV-2} RNA (Esg), indicator of active viral replication (Chandrashekar et al., 2020; Tostanoski et al., 2020; Wolfel et al., 2020), showed absence of replicating virus in the three vaccinated groups versus a mean±SD of (1.24±0.99)×10⁹ copies of Esg RNA of SARS-CoV-2/lungs in the sham-vaccinated group (FIG. 12E).

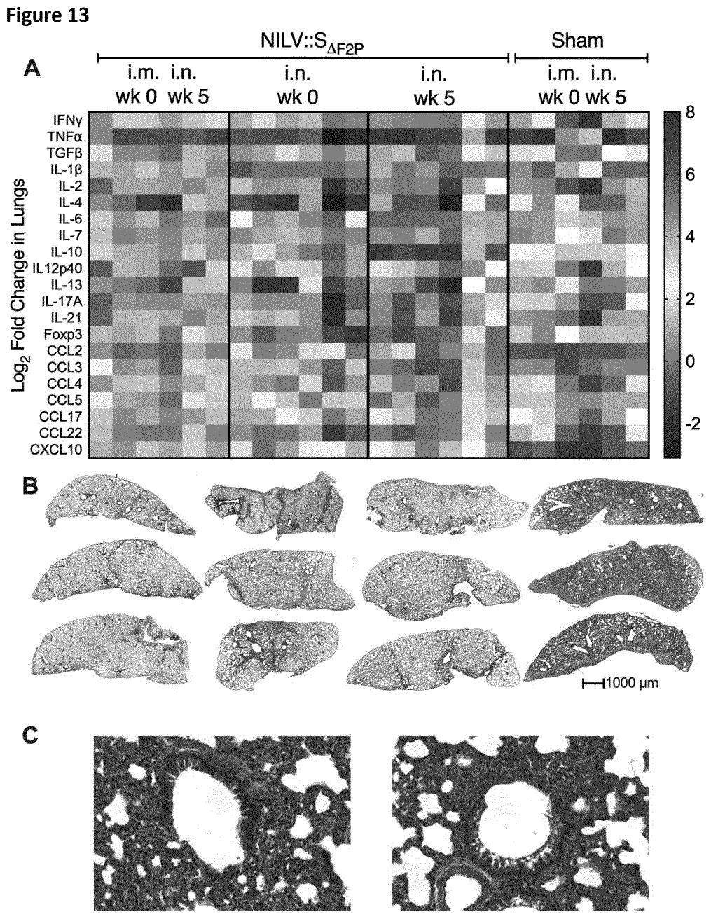

At 4 dpi, as evaluated by qRT-PCR in total lung homogenates, substantially decreased inflammation was detected in NILV::S_{ΔF2P}-vaccinated hamsters compared to their sham-vaccinated counterparts, regardless of the immunization regimen, i.e., i.m.-i.n. prime-boost or single i.n. injection given at wk 0 or 5 (FIG. 13A). Histopathological lung analysis showed that in the NILV::S_{ΔF2P}-immunized hamsters, pulmonary lesions were rare or undetectable, while in the sham-vaccinated controls, considerable parenchyma infiltration and consolidation, as well as marked alteration and effacement of bronchiolar epithelium were detected (FIG. 13B, C).

These data collectively indicated that a single i.n. administration of NILV::S_{ΔF2P} was as protective as a systemic prime and i.n. boost regimen, conferred sterilizing pulmonary immunity against SARS-CoV-2 and readily prevented lung inflammation and pathogenic tissue injury in the susceptible hamster model.

Altogether, based on a complete set of virological, immunological and expected histopathological data (the latter in progress), the LV::S_{FL} vector elicits S_{CoV-2}-specific nAbs and T-cell responses, correlative with substantial level of protection against SARS-CoV-2 infection in two pertinent animal models, and notably upon mucosal i.n. administration.

### Example 1.6: Discussion

Prophylactic strategies are necessary to control SARS-CoV-2 infection which, 6 months into the pandemic, still continue to spread exponentially without sign of slowing down. It is now demonstrated that primary infection with SARS-CoV-2 in rhesus macaques leads to protective immunity against re-exposure (Chandrashekar et al., 2020). Numerous vaccine candidates, based on naked DNA (Yu et al., 2020) or mRNA, recombinant protein, replicating or non-replicating viral vectors, including adenoviral Ad5 vector (Zhu et al., 2020), or alum-adjuvanted inactivated virus (Gao et al., 2020) are under active development for COVID-19 prevention. Our immunologic rationale for selecting LV vector to deliver gene encoding S_{CoV-2} antigen is based on the insights obtained on the efficacy of heterologous gene expression in situ, as well as the longevity and composite nature of humoral and cell-mediated immunity elicited by this immunization platform. Unique to LV is the ability to transduce proliferating and resting cells (Esslinger et al., 2002; He et al., 2005), thereby LV serves as a powerful vaccination strategy (Beignon et al., 2009; Buffa et al., 2006; Coutant et al., 2012; Gallinaro et al., 2018; Iglesias et al., 2006) to provokes strong and long-lasting adaptive responses. Notably, in net contrast to many other viral vectors, LV vectors do not suffer from pre-existing immunity in populations, which is linked to their pseudo-typing with the glycoprotein envelop from Vesicular Stomatitis Virus, in which humans are barely exposed. We recently demonstrated that a single injection of a LV expressing Zika envelop provides a rapid and durable protection against Zika infection (Ku et al., 2020). Our recent comprehensive systematic comparison of LV to the gold standard Ad5 immunization vector also documented the superior ability of LV to induce multifunctional and central memory T cells in the mouse model, and stronger immunogenicity in outbred rats (Ku et al., 2021 (PMID: 33357418), underlining the largely adapted properties of LV for vaccinal applications.

We evaluated the efficacy of LV each encoding one of the variants of S, i.e., full-length, membrane anchored (LV::S_{FL}), S1-S2 ecto-domain, devoid of the transmembrane and C-terminal short internal tail (LV::S1-S2), or S1 alone (LV::S1). Even though a single administration of each of these LV was able to induce high anti-S_{CoV-2} Ab titers, only LV::S_{FL} was able to induce highly functional nAbs. Such single-injection of LV-based vaccine induced a neutralizing activity, which on average was comparable to those found in a cohort of SARS-CoV-2 patients manifesting mild symptoms. This finding predicted a protective potential of the humoral responses induced by the LV::S_{FL} vector. In parallel, S-specific CD4+ and CD8⁺ T-cell responses were also observed in the spleen of mice as early as 2 weeks after a single LV::S_{FL} injection, as detectable against numerous MHC-I- or -II-restricted immunogenic regions that we identified in C57BL/6 (H-2^{b}) mice.

Linked to the absence of permissibility of laboratory mice to SARS-CoV-2 replication and the current unavailability of hACE2 transgenic mice in Europe, we set up an in vivo-infection murine model in which the hACE2 expression is induced in the respiratory tracts by an i.n. Ad5::hACE2 pretreatment prior to SARS-CoV-2 inoculation. This approach renders mice largely permissive to SARS-CoV-2 replication in the lungs and allows assessment of vaccine or drug efficacy against this virus. This method has also been successfully used to establish the expression of human DPP4 for the study of mouse infection with MERS-CoV (Zhao et al., 2014). Even though the Ad5::hACE2 model may not fully mimic the physiological ACE2 expression profile and thus may not reflect all the aspects of the pathophysiology of SARS-CoV-2 infection, it provides a pertinent model to evaluate in vivo the effects of anti-viral drugs, vaccine candidates, various mutations or genetic backgrounds on the SARS-CoV-2 replication. By using a low dose of Ad5::hACE2/mouse, no particular CD45⁺ cell recruitments were detectable at day 4 post instillation, indicative of an absence of Ad5-related inflammation before the inoculation of SARS-CoV-2.

In the transduced mouse model which allows high rate of SARS-CoV-2 replication, vaccination by a single i.p. administration of 1×10⁷ TU of LV::S_{FL}, 6 weeks before the virus inoculation, was sufficient to inhibit the viral replication by ˜1 log₁₀. Further boosting via the systemic route did not afford improved protection rate when compared to a single administration. However, priming by systemic route and boosting via mucosal route efficiently inhibited viral replication and avoided lung inflammation. Such protection was correlated with high titers of anti-S_{CoV-2} IgG and a strong neutralization activity in sera. S-specific T-cell responses were also detected in the spleen of LV::S_{FL}-immunized mice, as assessed by ELISPOT followed by stimulation of splenocytes with pools of overlapping 15-mer peptides. Much longer termed experiments in appropriate KO mice or adoptive immune cell transfer approaches are necessary to identify the immunological pathways that contribute to disease severity or protection against SARS-CoV-2. Both nAbs and cell-mediated immunity, together very efficaciously induced with the LV-based vaccine candidate, synergize to inhibit infection and viral replication.

Substantial degrees of protection against SARS-CoV-2 infection, accompanied by drastic reduction in mucosal inflammation and lung tissue damage, were observed in Mesocricetus auratus Golden hamsters immunized following prime-boost/target regimen with either integrative or non-integrative LV::S_{FL}. Confirmation of the protection results in this highly sensitive species further favors the LV::S_{FL} vaccine candidate, especially under its non-integrative variant, for future introduction into clinical trials.

Ab-Dependent Enhancement (ADE) of coronavirus entry to the host cells has been evoked as a mechanism which could be an obstacle in vaccination against coronaviruses. With DNA (Yu et al., 2020) or inactivated SARS-CoV-2 virus (Gao et al., 2020) vaccination in macaques, no immunopathological exacerbation has been observed but could not be excluded. Long term observation even after decrement in Ab titer could be necessary to exclude such hypothesis. In the case of MERS-CoV, it has been reported that one particular RBD-specific neutralizing monoclonal Ab (Mersmab1), by mimicking the viral receptor human DPP4 and inducing conformational rearrangements of S_{MERS}, can mediate in vitro ADE of MERS-CoV into the host cells (Wan et al., 2020). We believe that it is difficult to compare the polyclonal Ab response with its paratope repertoire complexity with the singular properties of a monoclonal Ab which cannot be representative of the polyclonal response induced by a vaccine. In addition, very contradictory results from the same team reported that a single-dose treatment with a humanized version of Mersmab1 afforded complete protection of a human transgenic mouse model from lethal MERS challenge (Qiu et al., 2016). Therefore, even with an Ab which could facilitate the cell host invasion in vitro in some conditions, not only there is no exacerbation of the infection in vivo, but also there is a notable protection. Indeed, to affirm that Abs could cause ADE in vivo, it is necessary, by large scale B-cell fusions, until they have made to estimate the probability of generation of such Ab.

Prophylactic vaccination is the most cost-effective and efficient strategy against infectious diseases and notably against emerging coronaviruses in particular. Our results provide strong evidences that the LV vector coding for SFS protein of SARS-CoV-2 used via the mucosal route of vaccination represent a promising vaccine candidate against COVID-19.

TABLE 1
Sequences of primers and probes for SARS-COV-2 viral load determination.

| Primer/Probe Name and | |
|---|---|
| SEQ ID No. | DNA Sequences |
| ″E-Sarbeco″ Fw-ID No. 34 | 5′-ACAGGTACGTTAATAGTTAATAGCGT-3′ |
| ″E-Sarbeco″ Rv-ID No. 35 | 5′-ATATTGCAGCAGTACGCACACA-3′ |
| ″E-Sarbeco″ Probe-ID | 5′-FAM-ACACTAGCCATCCTTACTGCGCTTCG-BHQ-1-3′ |
| No. 36 | |

TABLE 2
Sequences of primers used to quantitate mouse cytokines and
chemokines by qRT-PCR

| Gene and SEQ ID | |
|---|---|
| No. | Sequences |
| β-globin-ID No. 37 | F: 5′-ATGGGAAGCCGAACATACTG-3′ |
| -ID No. 38 | R: 5′-CAGTCTCAGTGGGGGTGAAT-3′ |
| GAPDH-ID No. 39 | F: 5′-TTCACCACCATGGAGAAGGC-3′ |
| -ID No. 40 | R: 5′-GGCATGGACTGTGGTCATGA-3′ |
| IFNα-ID No. 41 | F: 5′-GGATGTGACCTTCCTCAGACTC-3′ |
| -ID No. 42 | R: 5′-ACCTTCTCCTGCGGGAATCCAA-3′ |
| IFNγ-ID No. 43 | F: 5′-TCAAGTGGCATAGATGTGGAAGAA-3′ |
| -ID No. 44 | R: 5′-TGGCTCTGCAGGATTTTCATG-3′ |
| TNFα-ID No. 45 | F: 5′-CATCTTCTCAAAATTCGAGTGACAA-3′ |
| -ID No. 46 | R: 5′-TGGGAGTAGACAAGGTACAACCC-3′ |
| TGFβ-ID No. 47 | F: 5′-TGACGTCACTGGAGTTGTACGG-3′ |
| -ID No.48 | R: 5′-GGTTCATGTCATGGATGGTGC-3′ |
| IL1ß-ID No. 49 | F: 5′-TGGACCTTCCAGGATGAGGACA-3′ |
| -ID No.50 | R: 5′-GTTCATCTCGGAGCCTGTAGTG-3′ |
| IL2-ID No. 51 | F: 5′-CCTGAGCAGGATGGAGAATTACA-3′ |
| -ID No. 52 | R: 5′-TCCAGAACATGCCGCAGAG-3′ |
| IL4-ID No. 53 | F: 5′-CGAGGTCACAGGAGAAGGGA-3′ |
| -ID No. 54 | R: 5′-AAGCCCTACAGACGAGCTCACT-3′ |
| IL5-ID No. 55 | F: 5′-GATGAGGCTTCCTGTCCCTACT-3′ |
| -ID No. 56 | R: 5′-TGACAGGTTTTGGAATAGCATTTCC-3′ |
| IL6-ID No. 57 | F: 5′-CTGCAAGTGCATCATCGTTGTTC-3′ |
| -ID No. 58 | R: 5′-TACCACTTCACAAGTCGGAGGC-3′ |
| IL10-ID No. 59 | F: 5′-GGTTGCCAAGCCTTATCGGA-3′ |
| -ID No.. 60 | R: 5′-ACCTGCTCCACTGCCTTGCT-3′ |
| IL12p40-ID No. 61 | F: 5′-GGAAGCACGGCAGCAGAATA-3′ |
| -ID No. 62 | R: 5′-AACTTGAGGGAGAAGTAGGAATGG-3′ |
| IL17A-ID No. 63 | F: 5′-GAAGCTCAGTGCCGCCA-3′ |
| -ID No. 64 | R: 5′-TTCATGTGGTGGTCCAGCTTT-3′ |
| IL18-ID No. 65 | F: 5′-GACAGCCTGTGTTCGAGGATATG-3′ |
| -ID No. 66 | R: 5′-TGTTCTTACAGGAGAGGGTAGAC-3′ |
| IL33-ID No. 67 | F: 5′-CTACTGCATGAGACTCCGTTCTG-3′ |
| -ID No. 68 | R: 5′-AGAATCCCGTGGATAGGCAGAG-3′ |
| CCL2-ID No. 69 | F: 5′-AGGTCCCTGTCATGCTTCTG-3′ |
| -ID No. 70 | R: 5′-TCTGGACCCATTCCTTCTTG-3′ |
| CCL3-ID No. 71 | F: 5′-CCTCTGTCACCTGCTCAACA-3′ |
| -ID No. 72 | R: 5′-GATGAATTGGCGTGGAATCT-3′ |
| CCL5-ID No. 73 | F: 5′-GTGCCCACGTCAAGGAGTAT-3′ |
| -ID No. 74 | R: 5′-GGGAAGCGTATACAGGGTCA-3′ |
| CXCL5-ID No. 75 | F: 5′-GCATTTCTGTTGCTGTTCACGCTG-3′ |
| -ID No. 76 | R: 5′-CCTCCTTCTGGTTTTTCAGTTTAGC-3′ |
| CXCL9-ID No. 77 | F: 5′-AAAATTTCATCACGCCCTTG-3′ |
| -ID No. 78 | R: 5′-TCTCCAGCTTGGTGAGGTCT-3′ |
| CXCL10-ID No. 79 | F: 5′-GGATGGCTGTCCTAGCTCTG-3′ |
| -ID No. 80 | R: 5′-ATAACCCCTTGG GAAGATGG-3′ |

### Example 2: Generation of a Transgenic Mice Harboring the Human ACE2 Gene

To date several Transgenic (Tg) mice of different strains expressing the hACE2 gene under distinct transcription and expression control sequences have been provided, some of them originating from developments performed to fulfil needs that arose when on emergence of SARS-CoV epidemic in 2003. These earlier developed Tg mice and further models have been assessed for the study and understanding of the pathogenesis of SARS-CoV and have shown to be permissible to viral replication and sometimes to some degree of disease symptom or clinical illness but the observed various clinical profiles in Tg mice inoculated with SARS-CoV-2 have not yet provided proved suitable to reproduce all aspects of the outcome of the infection, in particular have not adequately shown virus spread as observed in human patients, in particular spread beyond the airways and the pulmonary tract, such as spread to the brain. Also the available Tg mice have not shown all the consistent disease symptoms that would reproduce the symptoms observed in human patients.

A B6.K18-ACE2^{2PrImn/JAX} mouse strain has been previously deposited at JAX Laboratories (Jackson Laboratories, Bar Harbor, Me.). However, the new B6.K18-hACE2^{IP-THV} transgenic mice that the inventors generated according to the present invention display distinctive characteristics identified following SARS-CoV-2 intranasal (i.n.) inoculation. In fact, in addition to the large permissibility of their lungs to SARS-CoV-2 replication and viral dissemination to peripheral organs, B6.K18-hACE2^{IP-THV} mice surprisingly allow substantial viral replication in the brain, which is ≈4 log₁₀ higher than the replication range observed in the previously available B6.K18-ACE2^{2PrImn/JAX} strain (McCray et al., 2007). This new mouse model, not only has broad applications in the study of COVID-19 vaccine or COVID-19 therapeutics efficacy, but also provides an experimental model to elucidate COVID-19 immune/neuro-physiopathology. Neurotropism of SARS-CoV-2 has been demonstrated and some COVID-19 human patients present symptoms like headache, confusion, anosmia, dysgeusia, nausea, and vomiting (Bourgonje et al., 2020). Olfactory transmucosal SARS-CoV-2 invasion is also very recently described as a port of central nervous system entry in human individuals with COVID-19 (https://doi.org/10.1038/s41593-020-00758-5). Since coronaviruses can infect the central nervous system (Bergmann et al., 2006), the B6.K18-hACE2^{IP-THV} small rodent experimental model represents an invaluable pre-clinical or co-clinical animal model of major interest for: (i) investigation of immune protection of the brain and (ii) exploration of COVID-19-derived neuropathology.

1. Construction of the Human Keratin 18 Promoter

The human K18 promoter (GenBank: AF179904.1 nucleotides 90 to 2579) was amplified by nested PCR from A549 cell lysate, as described previously (Chow et al., 1997; Koehler et al., 2000). The “i6x7” intron (GenBank: AF179904.1 nucleotides 2988 to 3740) was synthesized by Genscript. The “K18i6x7PA” promoter, previously used to generate B6.K18-ACE2^{2PrImn/JAX} strain, includes the K18 promoter, the “i6x7” intron at 5′ and an enhancer/polyA sequence (PA) at 3′ of the hACE2 gene. The^{K18 IP-ThV} promoter used here contains, instead of PA, the stronger wild-type Woodchuck Hepatitis Virus Posttranscriptional Regulatory Element (WPRE) at 3′ of the hACE2 gene. In contrast to K18i6x7PA construct which harbors the 3′ regulatory region containing a polyA sequence, the K18^{IP-ThV} construct takes benefice of the polyA sequence already present within the 3′ Long Terminal Repeats (LTR) of the pFLAP LV plasmid, used for transgenesis. The i6x7 intronic part was modified to introduce a consensus 5′ splicing donor and a 3′ donor site sequence. The AAGGGG (SEQ ID No.97) donor site was further modified for the AAGTGG (SEQ ID No.95) consensus site. Based on a consensus sequence logo (Dogan et al., 2007), the poly-pyrimidine tract preceding splicing acceptor site (TACAATCCCTC (SEQ ID No.82) in original sequence GenBank AF179904.1 and TTTTTTTTTTT (SEQ ID No.83) in K18^{JAX}) was replaced by CTTTTTCCTTCC (SEQ ID No.96) to limit incompatibility with the reverse transcription step during transduction. Moreover, original splicing acceptor site CAGAT was modified to correspond to the consensus sequence CAGGT (SEQ ID No.84). As a construction facility, a ClaI restriction site was introduced between the promoter and the intron. The construct was inserted into a pFLAP plasmid between the MluI and BamHI sites. The hACE2 cDNA was introduced between the BamHI and XhoI sites by restriction/ligation. Integrative LV::K18-hACE2^{IP-THV} was produced as described elsewhere (Sayes et al., 2018) and concentrated by two cycles of ultracentrifugation at 22,000 rpm for 1 h at 4° C.

2. Transgenesis

High tittered (8.32×10⁹ TU/ml) integrative LV::K18-hACE2^{IP-THV} was micro-injected into the pellucid area of fertilized eggs which were transplanted into pseudo-pregnant B6CBAF1 females (Charles Rivers). The NO mice were investigated for integration and copy number of hACE2 gene per genome by using hACE2-forward: 5′-TCC TAA CCA GCC CCC TGT T-3′ (SEQ ID No.85) and hACE2-reverse: 5′-TGA CAA TGC CAA CCA CTA TCA CT-3′ (SEQ ID No.86) primers in PCR applied on genomic DNA prepared from the tail biopsies. Toward stabilization of the progeny, transgene positive males were then crossed to WT C57BL/6 females (Charles Rivers). Transgene transfer by microinjection of integrative LV::K18-hACE2^{IP-THV} into the nucleus of fertilized eggs was particularly efficient. At the NO generation, 11% of the mice obtained, i.e., 15 out of 139, had at least one copy of the transgene per genome. Eight NO males carrying the transgene were crossed with female C57BL/6 WT mice (Janvier, Le Genest Saint Isle, France). At the N1 generation, ≈62% of the mice obtained, i.e., 91 out of 147, had at least one copy of the transgene per genome. 10 N1 males carrying the transgene were further crossed with female C57BL/6 WT mice.

During the immunization period female or male transgenic mice were housed in individually-ventilated cages under specific pathogen-free conditions. Mice were transferred into individually filtered cages in isolator for SARS-CoV-2 inoculation at the Institut Pasteur animal facilities. Prior to i.n. injections, mice were anesthetized by i.p. injection of Ketamine (Imalgene, 80 mg/kg) and Xylazine (Rompun, 5 mg/kg).

3. Genotyping and Quantitation of hACE2 Gene Copy Number/Genome in Transgenic Mice

Genomic DNA (gDNA) from transgenic mice was prepared from the tail biopsies by phenol-chloroform extraction. A 60 ng of gDNA were used as a template of qPCR with SyBr Green using specific primers listed in Table 3. Using the same template and in the similar reaction plate, mouse PKD1 (Polycystic Kidney Disease 1) and GAPDH were also quantified. All samples were run in quadruplicate in 10 μl reaction as follows: 10 in at 95° C., 40 cycles of 15 s at 95° C. and 30 sec at 60° C. To calculate the transgene copy number, the 2^{-ΔΔCt} method was applied using the PKD1 as a calibrator and GAPDH as a endogenous control. The 2^{-ΔΔCt} provides the fold change in copy number of the hACE2 gene relative to PKD1 gene.

TABLE 3
Sequences of primers used to genotype B6.K18-hACE2^{IP-THV}
transgenic mice.

| Primers and SEQ ID No. | |
|---|---|
| hACE2 Fw-SEQ ID No. 85 | TCCTAACCAGCCCCCTGTT |
| hACE2 Rv-SEQ ID No. 86 | TGACAATGCCAACCA CTATCACT |
| PKD1 Fw-SEQ ID No. 87 | GGCTGCTGAGCGTCTGGTA |
| PKD1 Rv-SEQ ID No. 88 | CCAGGTCCTGCGTGTCTGA |
| GAPDH-ACE2 Fw-SEQ ID No. 89 | GCCCAGAACATCATCCCTGC |
| GAPDH-ACE2 Rv-SEQ ID No. 90 | CCGTTCAGCTCTGGGATGACC |

4. K18-hACE2^{IP-THV} permissibility to SARS-CoV-2 replication

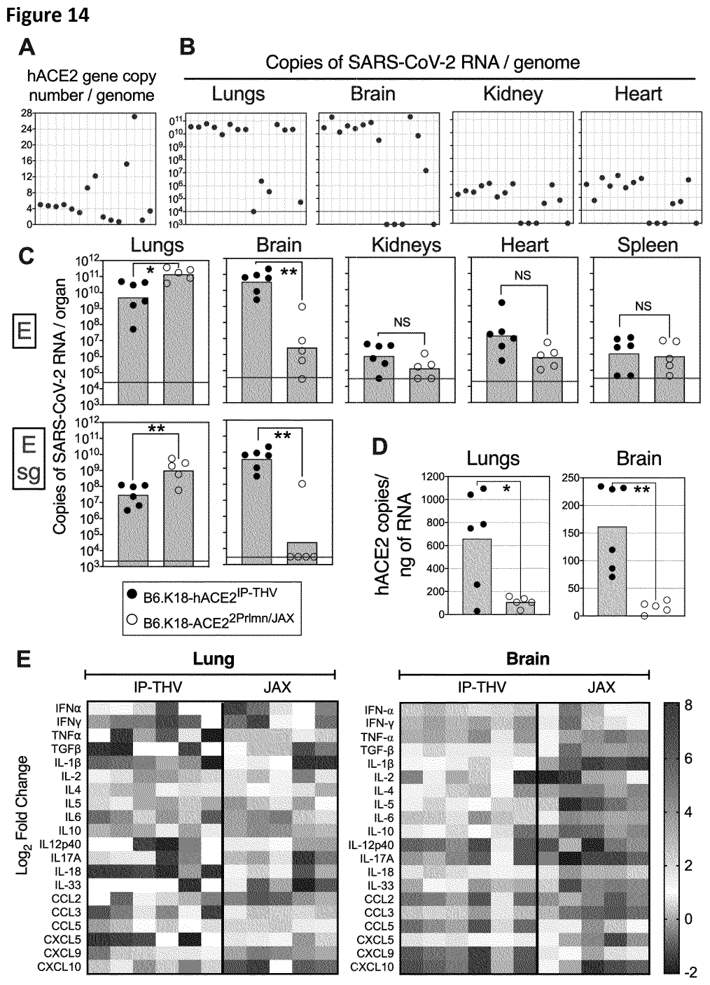

The permissibility of N1 mice to SARS-CoV-2 replication was evaluated in the sampled individuals from the progeny. N1 females with varying number of transgene copies per genome were sampled (FIG. 14A) and evaluated for their permissibility to SARS-CoV-2 replication (FIG. 14B). To do so, the selected mice were inoculated i.n. under general anesthesia with 0.3×10⁵ TCID₅₀ of the BetaCoV/France/IDF0372/2020 SARS-CoV-2 clinical isolate (Lescure et al., 2020), supplied by the National Reference Centre for Respiratory Viruses hosted by Institut Pasteur (Paris, France). The viral inoculum was contained in 20 μl for mice. Animals were then housed in an isolator in BioSafety Level 3 animal facilities of Institut Pasteur.

The organs recovered from the animals infected with live SARS-CoV-2 were manipulated following the approved standard operating procedures of these facilities.

At 3 days post-inoculation (dpi) the Mean±SD of lung viral loads were as high as (3.3±1.6)×10¹⁰ copies of SARS-CoV-2 RNA/mouse in the permissive mice (FIG. 14B). Note that the number of transgene copies per genome (FIG. 14A) was not proportional to the rate of SARS-CoV-2 replication in the lungs (FIG. 14B) and thus did not influence this phenotype. The amounts of lung viral loads were higher than those detected in positive control mice pre-treated i.n. with adenoviral vector serotype 5 encoding hCAE2 (Ad5::hACE2) that we previously described as a suitable model which also allows vaccine efficacy assay. Remarkably, substantial viral loads, i.e., (5.7±7.1)×10¹⁰ copies of SARS-CoV-2 RNA/mouse were also detected in the brain of the permissive mice (FIG. 14B). Virus dissemination was also observed, although to a lesser extent, in the heart and kidneys at this time point post virus inoculation.

5. Comparison of B6.K18-ACE2^{2PrImn/JAX} and K18-hACE2^{IP-THV} Strains in Terms of Permissibility to SARS-CoV-2 Replication

We further comparatively evaluated SARS-CoV-2 replication in lungs and brain and dissemination to various organs in B6.K18-hACE2^{IP-THV} and B6.K18-ACE2^{2PrImn/JAX} mice (FIG. 14C). The lung viral loads were lower, i.e., (2.1±2.2)×10¹⁰ copies of SARS-CoV-2 RNA/mouse, in B6.K18-hACE2^{IP-THV} mice, compared to (18.3±13.3)×10¹⁰ copies in B6.K18-ACE2^{2PrImn/JAX} mice. However, viral replication in the brain of B6.K18-hACE2^{IP-THV} mice, i.e. (7.4±7.9)×10¹⁰ copies of SARS-CoV-2 RNA/mouse, was substantially higher compared to (1.9±74.3)×10⁸ copies in their B6.K18-ACE2^{2PrImn/JAX} counterparts. Measurement of brain viral loads by qRT-PCR specific to subgenomic E_{CoV-2} mRNA (Esg), detected Mean±SD of (7.55±7.74)×10⁹ copies of SARS-CoV-2 RNA in B6.K18-hACE2^{IP-THV} mice and no viral replication in 4 out of 5 the B6.K18-ACE2^{2PrImn/JAX} mice. Nota that measurement of viral loads by qRT-PCR specific to subgenomic E_{CoV-2} mRNA (Esg), characterizes only the replicative/infectious SARS-CoV-2 viral particles. Therefore, high rate of SARS-CoV-2 replication and high loads of infectious viral particles in the brain are major distinctive phenotypes of the new B6.K18-hACE2^{IP-THV} strain. Comparison of the hACE2 mRNA expression performed by qRT-PCR in the brain showed much higher amounts of the transgene expression in the brain of B6.K18-hACE2^{IP-THV} mice compared to B6.K18-ACE2^{2PrImn/JAX} mice (FIG. 14C). This substantial difference between the cervical SARS-CoV-2 replication in the transgenic strains was corroborated with significantly higher hACE2 mRNA expression in the brain of B6.K18-hACE2^{IP-THV} mice (FIG. 14D). However, hACE2 mRNA expression in the lungs of B6.K18-hACE2IP-THV mice was also higher than in B6.K18-ACE2^{2PrImn/JAX} mice, which cannot explain the lower viral replication in the former. A trend towards higher viral loads was also observed in the kidneys and heart of B6.K18-hACE2^{IP-ThV} compared to B6.K18-ACE2^{2PrImn/JAX} mice, even though the differences did not reach statistical significance (FIG. 14C). A trend towards higher viral loads was also observed in the kidneys and heart of B6.K18-hACE2^{IP-ThV}, even though the differences did not reach statistical significance.

Correlative with the brain viral loads, much higher inflammation was detected by qRT-PCR in the brain of B6.K18-hACE2^{IP-THV} mice compared to B6.K18-ACE2^{2PrImn/JAX} mice, at 3 dpi, showing an immunological/inflammatory symptom in the central nervous system of the former, but not in the latter (FIG. 14C). In concordance with the lung viral loads, as evaluated by qRT-PCR applied to total lung homogenates, B6.K18-hACE2^{IP-THV} mice displayed less pulmonary inflammation than B6.K18-ACE2^{2PrImn/JAX} mice (FIG. 14E). Remarkably, this assay applied to total brain homogenates detected substantial degrees of inflammation in B6. K18-hACE2^{IP-THV} but not in B6.K18-ACE2^{2PrImn/JAX} mice (FIG. 14E). In addition, B6.K18-hACE2^{IP-THV} mice reached the humane endpoint between 3 and 4 dpi and therefore display a lethal SARS-CoV-2-mediated disease more rapidly than their B6.K18-ACE2^{2PrImn/JAX} counterparts {Winkler, 2020 #102}.

Therefore, large permissibility to SARS-CoV-2 replication at both lung and CNS, marked brain inflammation and rapid lethal disease are major distinctive features of this new B6.K18-hACE2^{IP-THV} transgenic model.

Ethical Approval of Animal Studies

In all Examples, experimentation on mice and hamsters was realized in accordance with the European and French guidelines (Directive 86/609/CEE and Decree 87-848 of 19 Oct. 1987) subsequent to approval by the Institut Pasteur Safety, Animal Care and Use Committee, protocol agreement delivered by local ethical committee (CETEA #DAP20007, CETEA #DAP200058) and Ministry of High Education and Research APAFIS #24627-2020031117362508 v1.

### Example 3: Full CNS and Lung Prophylaxis Against SARS-CoV-2 by Intranasal Lentivector Vaccination

Here, we generated a new hACE2 transgenic mouse strain with unprecedent permissibility of the brain to SARS-CoV-2 replication. By use of this unique preclinical animal model, we demonstrated the importance of i.n. booster immunization with this LV-based vaccine candidate to reach full protection of not only lungs but also CNS against SARS-CoV-2. Our results indicate that i.n. vaccination step with non-cytopathic and non-inflammatory LV, appears to be a performant and safe strategy to elicit sterilizing immunity in the main anatomical sites affected by COVID-19.

Methods

Construction and production of LV::S_{ΔF2P}

A codon-optimized S_{ΔF2P} sequence (1-1262) (SEQ ID No. 14). was amplified from pMK-RQ_S-2019-nCoV and inserted into pFlap by restriction/ligation between BamHI and XhoI sites, between the native human ieCMV promoter and a mutated Woodchuck Posttranscriptional Regulatory Element (WPRE) sequence. The atg starting codon of WPRE was mutated (mWPRE) to avoid transcription of the downstream truncated “X” protein of Woodchuck Hepatitis Virus for safety concerns (FIG. 17). Plasmids were amplified and used to produce LV as previously described in Example 1.

Mice

Transgenic mice were generated as disclosed in detail in Example 2.

Humoral and T-Cell Immunity, Inflammation

As recently detailed elsewhere (Ku et al., 2021), T-splenocyte responses were quantitated by IFN-g ELISPOT and anti-S IgG or IgA Abs were detected by ELISA by use of recombinant stabilized S_{CoV-2}. NAb quantitation was performed by use of S_{CoV-2} pseudo-typed LV, as recently described (Anna et al., 2020; Sterlin et al., 2020). The qRT-PCR quantification of inflammatory mediators in the lungs and brain of hamsters and mice was performed in total RNA extracted by TRIzol reagent, as detailed in Example 1.

SARS-CoV-2 Inoculation

Hamsters or transgenic B6.K18-hACE2^{IP-THV} or B6.K18-ACE2^{2PrImn/JAX} were anesthetized by i.p. injection of mixture Ketamine and Xylazine, transferred into a biosafety cabinet 3 and inoculated i.n. with 0.3×10⁵ TCID₅₀ of the BetaCoV/France/IDF0372/2020 SARS-CoV-2 clinical isolate (Lescure et al., 2020). This clinical isolate was a gift of the National Reference Centre for Respiratory Viruses hosted by Institut Pasteur (Paris, France), headed by Pr. van der Werf. The human sample from which this strain was isolated has been provided by Dr. Lescure and Pr. Yazdanpanah from the Bichat Hospital, Paris, France. The viral inoculum was contained in 20 μl for mice and in 50 μl for hamsters. Animals were housed in an isolator in BioSafety Level 3 animal facilities of Institut Pasteur. The organs recovered from the infected animals were manipulated according to the approved standard procedures of these facilities.

Determination of Viral Loads in the Organs

Organs from mice or hamsters were removed aseptically and immediately frozen at −80° C. RNA from circulating SARS-CoV-2 was prepared from lungs as recently described (Ku et al). Briefly, lung homogenates were prepared by thawing and homogenizing of the organs using lysing matrix M (MP Biomedical) in 500 μl of ice-cold PBS in an MP Biomedical Fastprep 24 Tissue Homogenizer. RNA was extracted from the supernatants of lung homogenates centrifuged during 10 min at 2000 g. Alternatively, total RNA was prepared from lungs or other organs by addition of lysing matrix D (MP Biomedical) containing 1 mL of TRIzol reagent and homogenization at 30 s at 6.0 m/s twice using MP Biomedical Fastprep 24 Tissue Homogenizer. Total RNA was extracted using TRIzol reagent (ThermoFisher). SARS-CoV-2 E gene (Corman et al., 2020) or E sub-genomic mRNA (sgmRNA) (Wolfel et al., 2020), was quantitated following reverse transcription and real-time quantitative TaqMan® PCR, using SuperScript™ Ill Platinum One-Step qRT-PCR System (Invitrogen) and specific primers and probe (Eurofins) (Table 4). The standard curve of EsgmRNA assay was performed using in vitro transcribed RNA derived from PCR fragment of “T7 SARS-CoV-2 E-sgmRNA”. The in vitro transcribed RNA was synthesized using T7 RiboMAX Express Large Scale RNA production system (Promega) and purified by phenol/chloroform extraction and two successive precipitations with isopropanol and ethanol. Concentration of RNA was determined by optical density measurement, diluted to 10⁹ genome equivalents/μL in RNAse-free water containing 100 μg/mL tRNA carrier, and stored at −80° C. Serial dilutions of this in vitro transcribed RNA were prepared in RNAse-free water containing 10 μg/ml tRNA carrier to build a standard curve for each assay. PCR conditions were: (i) reverse transcription at 55° C. for 10 min, (ii) enzyme inactivation at 95° C. for 3 min, and (iii) 45 cycles of denaturation/amplification at 95° C. for 15 s, 58° C. for 30 s. PCR products were analyzed on an ABI 7500 Fast real-time PCR system (Applied Biosystems).

TABLE 4
Sequences of primers used to quantitate SARS-COV-2 loads by qRT-
PCR

| Primer/Probe | |
|---|---|
| SEQ ID No. | DNA Sequence |
| ″E-Sarbeco″ | 5′-ACAGGTACGTTAATAGTTAATAGCGT-3′ |
| Fw ID No. 91 | |
| ″E-Sarbeco″ | 5′-ATATTGCAGCAGTACGCACACA-3′ |
| Rv ID No. 92 | |
| ″E-Sarbeco″ | 5′-FAM-ACACTAGCCATCCTTACTGCGCTTCG-BHQ-1-3′ |
| ID No. 93 | |
| ″E-sgmRNA″ Fw | 5′-CGATCTCTTGTAGATCTGTTCTC-3′ |
| ID No. 94 | |

Cytometric Analysis of Immune Lung and Brain Cells

Isolation and staining of lung innate immune cells were largely detailed in Example 1. Cervical lymph nodes, olfactory bulb and brain from each group of mice were pooled and treated with 400 U/ml type IV collagenase and DNase I (Roche) for a 30-minute incubation at 37° C. Cervical lymph nodes and olfactory bulbs were then homogenized with glass homogenizer while brains were homogenized by use of GentleMacs (Miltenyi Biotech). Cell suspensions were then filtered through 100 μm-pore filters, washed and centrifuged at 1200 rpm during 8 minutes. Cell suspensions from brain were enriched in immune cells on Percoll gradient after 25 min centrifugation at 1360 g at RT. The recovered cells from lungs were stained as recently described elsewhere (Ku et al., 2021). The recovered cells from brain were stained by appropriate mAb mixture as follows. (i) To detect innate immune cells: Near IR Live/Dead (Invitrogen), FcγII/III receptor blocking anti-CD16/CD32 (BD Biosciences), BV605-anti-CD45 (BD Biosciences), PE-anti-CD11b (eBioscience), PE-Cy7-antiCD11c (eBioscience), (ii) to detect NK, neutrophils, Ly-6C^{+/-} monocytes and macrophages: Near IR DL (Invitrogen), FcγII/III receptor blocking anti-CD16/CD32 (BD Biosciences), BV605-anti-CD45 (BD Biosciences), PE-anti-CD11b (eBioscience), LPE-Cy7-antiCD11c (eBioscience), APC-anti-Ly6G (Miltenyi), BV711-anti-Siglec-F (BD), AF700-anti-NKp46 (BD Biosciences), FITC-anti-Ly6C (Abcam) (iii) To detect adaptive immune cells: Near IR Live/Dead (Invitrogen), FcγII/III receptor blocking anti-CD16/CD32 (BD Biosciences), APC-anti-CD45 (BD), PerCP-Cy5.5-anti-CD3 (eBioscience), FITC-anti-CD4 (BD Pharmingen), BV711-anti-CD8 (BD Horizon), BV605-anti-CD69 (Biolegend), PE-anti-CCR7 (eBioscience) and VioBlue-Anti-B220 (Miltenyi).Cells were incubated with appropriate mixtures for 25 minutes at 4° C., washed in PBS containing 3% FCS and fixed with Paraformaldehyde 4% by an overnight incubation at 4° C. Samples were acquired in an Attune NxT cytometer (Invitrogen) and data analyzed by FlowJo software (Treestar, OR, USA).

Results

New hACE2 Transgenic Mice with Substantial Brain Permissibility to SARS-CoV-2 replication

B6.K18-hACE2^{IP-THV} mice were generated as disclosed in Example 2. The permissibility of these mice to SARS-CoV-2 replication was evaluated and it was determined that large permissibility to SARS-CoV-2 replication at both lung and CNS, marked brain inflammation and rapid lethal disease are major distinctive features of this new B6.K18-hACE2^{IP-THV} transgenic model.

Full Protection of Lungs and Brain in LV::S_{ΔF2P}-Immunized B6.K18-hACE2^{IP-THV} Mice

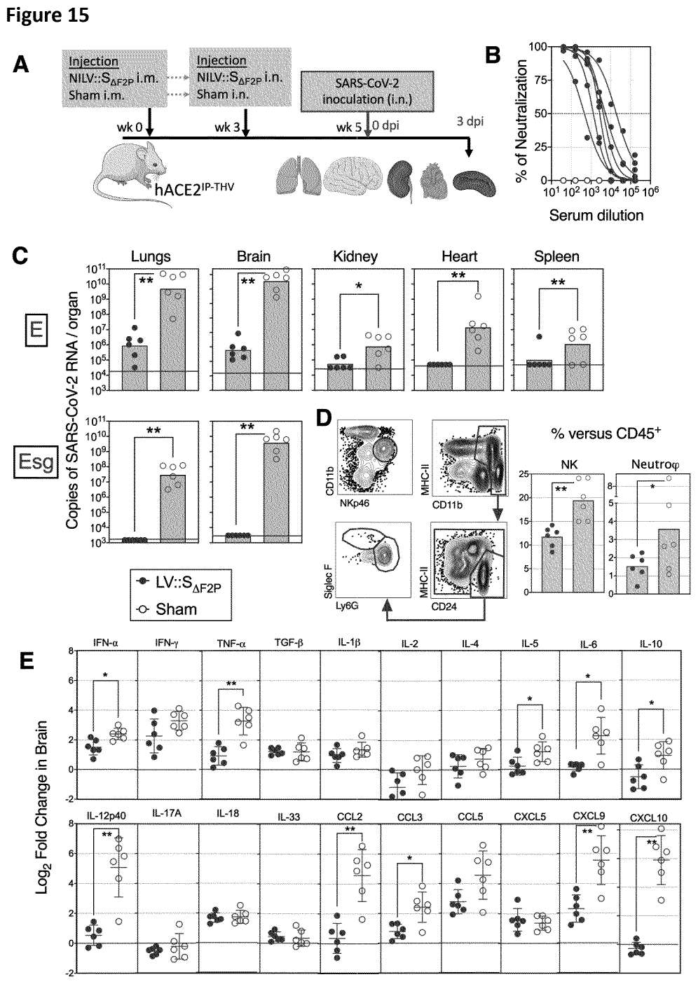

We then evaluated the vaccine efficacy of LV::S_{ΔF2P} in B6.K18-hACE2^{IP-THV} mice. Individuals (n=6/group) where primed i.m. with 1×10⁷ TU/mouse of LV::S_{ΔF2P} or an empty LV (sham) at wk 0 and then boosted i.n. at wk 3 with the same dose of the same vectors (FIG. 15A). Mice were then challenged with SARS-CoV-2 at wk 5. A high serum neutralizing activity, i.e., EC50 mean±SD of 5466±6792, was detected in LV::S_{ΔF2P}-vaccinated mice (FIG. 15B). This vaccination conferred substantial degrees of protection against SARS-CoV-2 replication, not only in the lungs, but also in the brain (FIG. 15C). Notably, quantitation of brain viral loads by Esg qRT-PCR detected no copies of this replication-related SARS-CoV-2 RNA in LV::S_{ΔF2P}-vaccinated mice versus (7.55±7.84)×10⁹ copies in the brain of the sham-vaccinated controls.

At 3 dpi, cytometric investigation of the lung innate immune cell subsets (FIG. 15D,) detected significant decrease in the proportions of NK cells and neutrophils inside the lung CD45+ cells in the LV::S_{ΔF2P}-vaccinated B6.K18-hACE2^{IP-THV} mice, compared to the sham-vaccinated controls (FIG. 15D). At 3 dpi, as evaluated by qRT-PCR applied to brain homogenates, NILV::S_{ΔF2P}-vaccinated B6.K18-hACE2^{IP-THV} mice had significant decreases in the expression levels of IFN-□, TNF-□, IL-5, IL-6, IL-10, IL-12p40, CCL2, CCL3, CXCL9 and CXCL10, compared to the sham group (FIG. 15E). No noticeable changes in the lung inflammation were recorded between the two groups (not shown).

Therefore, an i.m.-i.n. prime-boost with NILV::S_{ΔF2P} prevents SARS-CoV-2 replication in both lung and CNS anatomical areas and inhibits virus-mediated lung pathology and neuro-inflammation.

Requirement of i.n. Boost for Full Protection of Brain in B6.K18-hACE2^{IP-THV} Mice

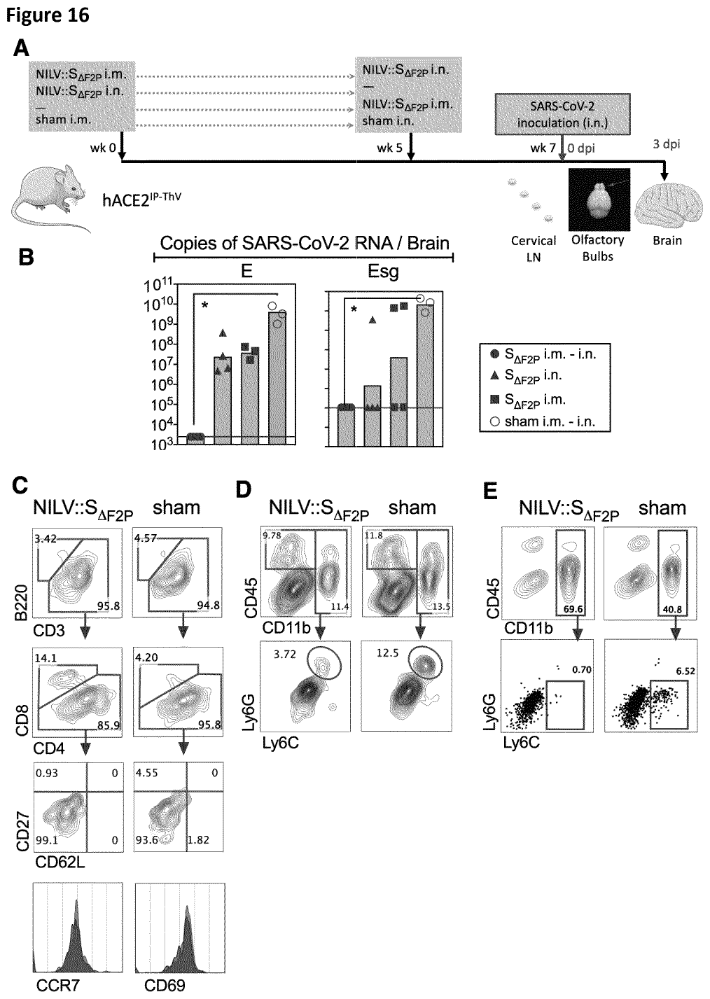

To go further in characterization of the protective properties of LV, in the following experiments in B6.K18-hACE2^{IP-THV} mice, similar to the hamster model, we used the non-integrative version of LV. The observed protection of brain against SARS-CoV-2 may reflect the benefits of i.n. route of LV administration against this respiratory and neurotropic virus. To address this hypothesis, B6.K18-hACE2^{IP-THV} mice were vaccinated with NILV::S_{ΔF2P}: (i) i.m. wk 0 and i.n. wk5, as a positive control, (ii) i.n. wk 0, or (iii) i.m. wk 5. Sham-vaccinated controls received i.n. an empty NILV at wks 0 and 5 (FIG. 16A). Mice were then challenged with SARS-CoV-2 at wk 7 and viral loads were determined in the brain s by E or Esg specific qRT-PCR at 3 dpi (FIG. 16B). In this highly stringent pre-clinical model, even performant, a single i.n. or i.m. injection of NILV::S_{ΔF2P} did not induce full protection in all animals of each group. Only i.m. prime followed by i.n. boost conferred full protection in all animals, showing the requirement of i.n. boost to reach full protection of brain.

As analyzed by cytometry, composition of innate and adaptive immune cells in the cervical lymph nodes were unchanged in NILV::S_{ΔF2P} i.m.-i.n. protected group, sham i.m.-i.n. unprotected group and untreated controls (data not shown). Notably, we detected increased proportion of CD8⁺ T cells in the olfactory bulb of NILV::S_{ΔF2P} i.m.-i.n. protected group compared to unprotected group (FIG. 16C). CD4⁺ T cells in the olfactory bulb had no distinctive activated or migratory phenotype, based on their expression of CD69 or CCR7, respectively. We detected increased amount of neutrophils in the olfactory bulb (FIG. 16D) and of CD11 b⁺ Ly6G⁻ Ly6C⁺ inflammatory monocytes in the brain (FIG. 16E) of unprotected mice, compared to NILV::S_{ΔF2P} i.m.-i.n. protected group, as a biomarker of inflammation and/or correlated with active viral replication.

Collectively, our data generated in the highly stringent B6.K18-hACE2^{IP-THV} mouse model support the advantage of NILV::S_{ΔF2P} i.n. boost in the immune protection of CNS from SARS-CoV-2 replication and the resulting infiltration and neuro-inflammation. The local induction and/or activation of mucosal immune response in nasal cavity and olfactory bulbs, i.e. the entry point for the virus, is a performant strategy.

Discussion

LV-based platform emerges as a powerful vaccination approach against COVID-19, notably when used in systemic prime followed by mucosal i.n. boost, able to induce sterilizing immunity against lung SARS-CoV-2 infection in preclinical animal models. We first demonstrate that a single i.n. administration of an LV encoding the S_{ΔF2P} prefusion form of S_{CoV-2} confers, as efficiently as an i.m.-i.n. prime-boost regimen, full protection of respiratory tracts in the highly susceptible hamster model, as evaluated by virological, immunological and histopathological parameters. The hamster ACE2 ortholog interacts efficaciously with S_{CoV-2}, which readily allows host cell invasion by SARS-CoV-2 and its high replication rate. With rapid weight loss and development of severe lung pathology subsequent to SARS-CoV-2 inoculation, this species provides a sensitive model to evaluate the efficacy of drug or vaccine candidates, for instance compared to Rhesus macaques which develop only a mild COVID-19 pathology (Munoz-Fontela et al., 2020; Sia et al., 2020). The fact that a single i.n. LV administration, either seven or two weeks before SARS-CoV-2 challenge, elicits sterilizing protection in this susceptible model is valuable in setting the upcoming clinical trials with this LV-based vaccine and could provide remarkable socio-economic advantages for mass vaccination.

To further investigate the efficacy of our vaccine candidates, we generated a new transgenic mouse model, by use of an LV-based transgenesis approach (Nakagawa and Hoogenraad, 2011). The ILV used in this strategy encodes for hACE2 controlled by cytokeratin K18 promoter, i.e., the same promoter as previously used by Perlman's team to generate B6.K18-ACE2^{2PrImn/JAX} mice (McCray et al., 2007), with a few adaptations to the lentiviral FLAP transfer plasmid. However, the new B6.K18-hACE2^{IP-THV} mice have certain distinctive features, as they express much higher levels of hACE2 mRNA in the brain and display markedly increased brain permissibility to SARS-CoV-2 replication, in parallel with a substantial brain inflammation and development of a lethal disease in <4 days post infection. These distinct characteristics can result from differential hACE2 expression profile due to: (i) alternative insertion sites of ILV into the chromosome compared to naked DNA, and/or (ii) different effect of the Woodchuck Posttranscriptional Regulatory Element (WPRE) versus the alfalfa virus translational enhancer (McCray et al., 2007), in B6.K18-hACE2^{IP-THV} and B6.K18-ACE2^{2PrImn/JAX} animals, respectively. Other reported hACE2 humanized mice express the transgene under: (i) murine ACE2 promoter, without reported hACE2 mRNA expression in the brain (Yang et al., 2007), (ii) “hepatocyte nuclear factor-3/forkhead homologue 4” (HFH4) promoter, i.e., “HFH4-hACE2” C3B6 mice, in which lung is the principal site of infection and pathology (Jiang et al., 2020; Menachery et al., 2016), and (iii) “CAG” mixed promoter, i.e. “AC70” C3H×C57BL/6 mice, in which hACE2 mRNA is expressed in various organs including lungs and brain (Tseng et al., 2007). Comparison of AC70 and B6.K18-hACE2^{IP-THV} mice may be informative to assess similarities and distinctions of these two models. However, here we report much higher brain permissibility of B6.K18-hACE2^{IP-THV} mice to SARS-CoV-2 replication, compared to B6.K18-ACE2^{2PrImn/JAX} mice. The B6.K18-hACE2^{IP-THV} murine model not only has broad applications in COVID-19 vaccine studies, but also provides a unique rodent model for exploration of COVID-19-derived neuropathology. Based on the substantial permissibility of the brain to SARS-CoV-2 replication and development of a lethal disease by these transgenic mice, this pre-clinical model can be considered as more stringent than the golden hamster model.

In this study, the use of the highly stringent B6.K18-hACE2^{IP-THV} mice demonstrated the importance of i.n. booster immunization for the induction of sterilizing protection of CNS by the LV-based vaccine candidate developed against SARS-CoV-2. Olfactory bulb may control viral CNS infection through the action of local innate and adaptive immunity (Durrant et al., 2016), and we observed increased frequencies of CD8⁺ T cells at this anatomically strategic area in i.m.-i.n. vaccinated and protected mice. Substantial reduction in the inflammation mediators was also demonstrated in the brain of these vaccinated and protected mice, together with decrease in the neutrophils and inflammatory monocytes in the olfactory bulbs and brain, respectively.

The source of neurological manifestations associated with COVID-19 in patients with comorbid conditions can be: (i) direct impact of SARS-CoV-2 on CNS, (ii) infection of brain vascular endothelium and, (iii) uncontrolled anti-viral immune reaction inside CNS. ACE2 is expressed in human neurons, astrocytes and oligodendrocytes, located in middle temporal gyrus and posterior cingulate cortex, which may explain the brain permissibility to SARS-CoV-2 in patients (Song et al., 2020; Hu et al., 2020). Viruses can invade the brain through neural dissemination or hematogenous route (Bohmwald et al., 2018; Desforges et al., 2019, 2014). The olfactory system establishes a direct connection to the CNS via frontal cortex (Mori et al., 2005). Neural transmission of viruses to the CNS can occur as a result of direct neuron invasion through axonal transport in the olfactory mucosa. Subsequent to intraneuronal replication, the virus spreads to synapses and disseminate to anatomical CNS zones receiving olfactory tract projections (Koyuncu et al., 2013; Zubair et al., 2020; Berth, 2009; Koyuncu et al., 2013; Roman et al., 2020). However, the detection of viral RNA in CNS regions without connection with olfactory mucosa suggests existence of another viral entry into the CNS, including migration of SARS-CoV-2-infected immune cells crossing the hemato-encephalic barrier or direct viral entry pathway via CNS vascular endothelium (Meinhardt et al., 2020). Although at steady state, viruses cannot penetrate to the brain through an intact blood-brain barrier (Berth, 2009), inflammation mediators which are massively produced during cytokine/chemokine storm, notably TNF-α and CCL2, can disrupt the integrity of blood-brain barrier or increase its permeability, allowing paracellular blood-to-brain transport of the virus or virus-infected leukocytes {Aghagoli, 2020 #77; Hu, 2011 #15}. Regardless of the mechanism of the SARS-CoV-2 entry to the brain, we provide evidence of the full protection of the CNS against SARS-CoV-2 by i.n. booster immunization with NILV::S_{ΔF2P}.

We reported results in Example 1 demonstrating the strong prophylactic capacity of LV::S_{FL} at inducing sterilizing protection in the lungs against SARS-CoV-2 infection. In the present study, moving toward clinical assay, we used LV encoding stabilized prefusion S_{ΔF2P} forms of S_{CoV-2} as an additional form of the S protein exhibiting vaccinal interest. This choice was based on data indicating that stabilization of viral envelop glycoproteins at their prefusion forms improve the yield of their production as recombinant proteins in industrial manufacturing of subunit vaccines, and the efficacy of nucleic acid-based vaccines by raising availability of the antigen under its optimal immunogenic shape (Hsieh et al., 2020). The prefusion stabilization approach has been so far applied to S protein of several coronaviruses, including HKU1-CoV, SARS-CoV, and MERS-CoV. Stabilized S_{MERS-CoV} has been shown to elicit much higher NAb responses and protection in pre-clinical animal models (Hsieh et al., 2020).

The sterilizing protection of the lungs conferred by a single i.n. administration and the full protection of CNS conferred by i.n. boost is an asset of primary importance. The non-cytopathic and non-inflammatory LV encoding either full length, or stabilized forms of S_{CoV-2}, from either ancestral or emerging variants of SARS-CoV-2 provides a promising COVID-19 vaccine candidate of second generation. Protection of the brain, so far not directly addressed by other vaccine strategies, has to be taken into account, considering the multiple and sometimes severe neuropathological manifestations associated with COVID-19.

### Example 4: Complete Cross-Protection Induced by NILV::S_{CoV-2} Wuhan Against the Genetically Distant P.1 (so Called Manaus, Brazil or γ) Variant

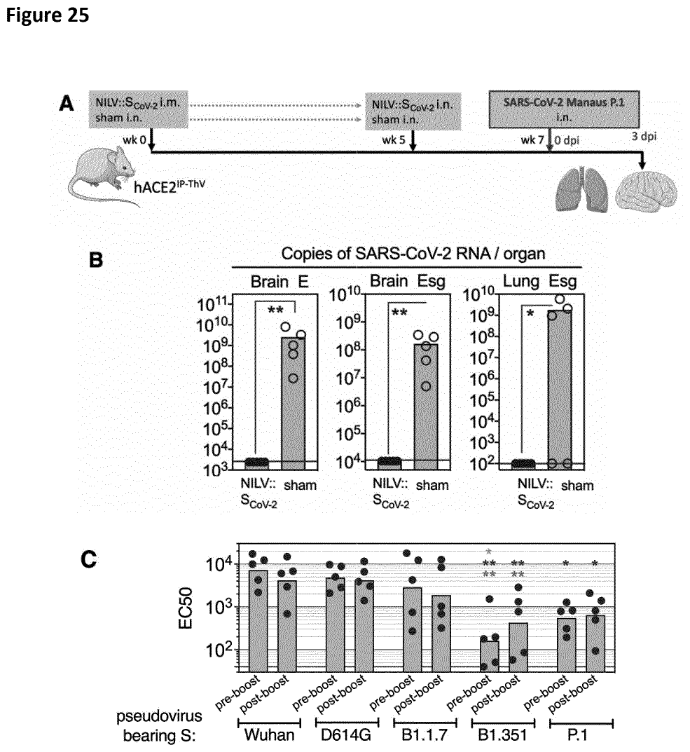

A critical issue regarding the COVID-19 vaccines currently in use is the protective potency against emerging variants. To assess this question with the NILV::S_{CoV-2} Wuhan vaccine candidate, B6.K18-hACE2^{IP-THV} transgenic mice were primed i.m. (wk0) and boosted i.n. (wk5) with NILV::S_{CoV-2} or sham (FIG. 25A). Mice were then challenged i.n. at wk 7 with 0.3×10⁵ TCID₅₀/mouse of P.1 (so called Manaus, Brazil, or γ) SARS-CoV-2, which is the most genetically distant variant, so far described (Buss et al., 2021). Determination of the brain and lung viral loads at 3 dpi demonstrated that i.m.-i.n. prime-boost with NILV::S_{CoV-2} Wuhan induced full cross protection of the brain and lungs against this genetically distant P.1 variant (FIG. 25B). We observed a markedly decreased ability of the sera of the NILV::S_{CoV-2 Wuhan}-vaccinated mice to neutralize S_{B1.351 or} S_{Manaus P.1} pseudo-viruses, compared to S_{Wuhan}, S_{D614G} or S_{B1.117} pseudo-viruses (FIG. 25C).

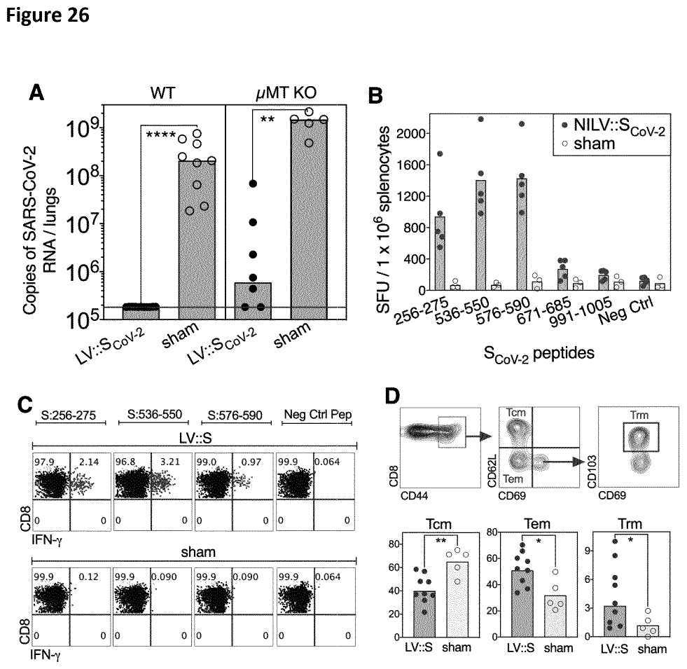

This drastically reduced protective B-cell response despite the remarkable protection, raised the possibility of T-cell involvement in this NILV::S_{CoV-2 Wuhan}-mediated full protection. To evaluate this possibility, we vaccinated following the same protocol (FIG. 25A), C57BL/6 WT or μMT KO mice. The μMT KO mice are deficient in mature B-cell compartment and therefore lack Ig/antibody response (Kitamura et al., 1991). To make these non-transgenic mice permissive to SARS-CoV-2 replication, they were pre-treated 4 days before the SARS-CoV-2 challenge with 3×10⁸ IGU of an adenoviral vector serotype 5 encoding hACE2 (Ad5::hACE2), as we previously described (Ku et al., 2021). Determination of lung viral loads at 3 dpi showed complete protection of the lungs in vaccinated WT mice as well as a highly significant protection in vaccinated μMT KO mice (FIG. 26A). This observation indicates that B-cell independent and antigen-specific cellular immunity, i.e., T-cell response, plays a major role in NILV::S_{CoV-2}-mediated protection against SARS-CoV-2.

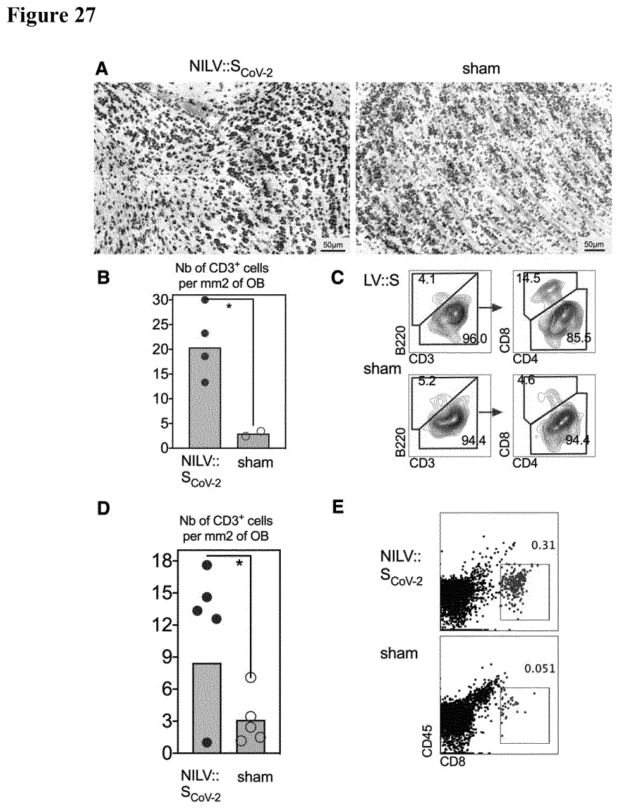

This is consistent with: (i) strong CD8⁺ T-cell responses induced by NILV::S_{CoV-2 Wuhan} at the systemic level (FIG. 26B), (ii) notable proportions of IFN-γ-producing lung CD8⁺ T cells, specific to several S_{CoV-2} epitopes, (FIG. 26C), (iii) high proportions of lung CD8⁺ T cells with effector memory (Tem) and resident memory (Trm) phenotype (FIG. 26D), (iv) recruitment of CD8⁺ T cells in the olfactory bulbs, detectable in mice vaccinated and challenged with SARS-CoV-2 Wuhan (FIG. 27A-C) or SARS-CoV-2 P.1 variant (FIG. 27D, E).

Remarkably, all murine and human CD8⁺ T-cell epitopes identified on S_{CoV-2 Wuhan} sequence are preserved in the mutated S_{CoV-2 Manaus P.1} (Table 5). These observations indicate the strong potential of NILV at inducing full protection of lungs and brain against ancestral and emerging SARS-CoV-2 variants by eliciting marked B and T cell-responses. In contrast to the B-cell epitopes which are targets of NAbs (Hoffmann et al., 2021), the so far identified T-cell epitopes have not been impacted by mutations accumulated in the S_{CoV-2} of the emerging variants.

TABLE 5
S_{CoV-2}-derived murine and human T-cell epitopes

| | | SEQ | a.a substitution/ |
|---|---|---|---|
| Murine | Sequence aa | ID NO: | deletion |
| H-2D^{b} | LDSKVGGNYNYLYRL | 18 | |
| H-2D^{b} | NKCVNFNFNGLTGTG | 16 | |
| H-2D^{b} | VRDPQTLEILDITPC | 17 | |
| H-2D^{b} | CASYQTQTNSPRRAR | 19 | P → Hin B1.1.7 |
| H-2D^{b} | VQIDRLITGRLQSLQ | 20 | |
| | Identified | | |
| Human | (Immundex data base) | | observation |
| A*0101 | LTDEMIAQY | 121 | |
| A*0201 | FLHVTYVPA | 122 | |
| A*0201 | KIYSKHTPI | 123 | |
| A*0201 | KLPDDFTGCV | 124 | |
| A*0201 | LLFNKVTLA | 125 | |
| A*0201 | RLDKVEAEV | 126 | |
| A*0201 | RLITGRLQSL | 127 | |
| A*0201 | RLQSLQTYV | 128 | |
| A*0201 | TLDSKTQSL | 129 | |
| A*0201 | VLNDILSRL | 130 | S → A in B1.1.7 |
| A*0201 | YLQPRTFLL | 131 | |
| A*0201 | RLNEVAKNL | 132 | |
| A*0201 | VVFLHVTYV | 133 | |
| A*0201 | NLNESLIDL | 134 | |
| A*0201 | FIAGLIAIV | 135 | |
| A*0301 | KCYGVSPTK | 136 | |
| A*0301 | GVYFASTEK | 137 | |
| A*1101 | RLFRKSNLK | 138 | |
| A*1101 | GTHWFVTQR | 139 | |
| A*1101 | GVYFASTEK | 137 | |
| A*2402 | KWPWYIWLGF | 140 | |
| A*2402 | QYIKWPWYI | 141 | |
| A*2402 | NYNYLYRLF | 142 | |
| A*2402 | RFDNPVLPF | 143 | D → A in B1.351 |
| B*0702 | SPRRARSVA | 144 | P → H in B1.1.7 |
| B*0702 | APHGVVFL | 145 | |
| B*3501 | QPTESIVRF | 146 | |
| B*3501 | LPFNDGVYF | 147 | |
| B*3501 | IPFAMQMAY | 148 | |
| B*4403 | YEQYIKWPW | 149 | |
| DR | ITRFQTLLALHRSYL | 150 | LAL deletion in |
| | | | B1.351 |
| DR | FNGLTVLPPLLTDEM | 151 | |
| DRB1*0101 | QLIRAAEIRASANLAATK | 152 | A → I in P.1 |
| DRB1*0401 | | | |
| DRB1*0701 | | | |
| DRB1*1501 | | | |

### Example 5: Identification of Spike from SARS-CoV-2 B1.351 (so Called South African or β) Variant as the Most Suitable Antigen for a Broad Protection LV Vaccine

As demonstrated in Example 4, we showed that NI LV::S_{CoV-2 Wuhan} largely protects the strongly susceptible B6. K18-hACE2^{IP-THV} transgenic mice against both the ancestral Wuhan and the most genetically distant Manaus P.1 SARS-CoV-2 variants. For the establishment of a therapeutic, to further improve the antigen, the use of the most suitable Spike variant, which can best consider the dynamics of the virus propagation of the known variants was considered.

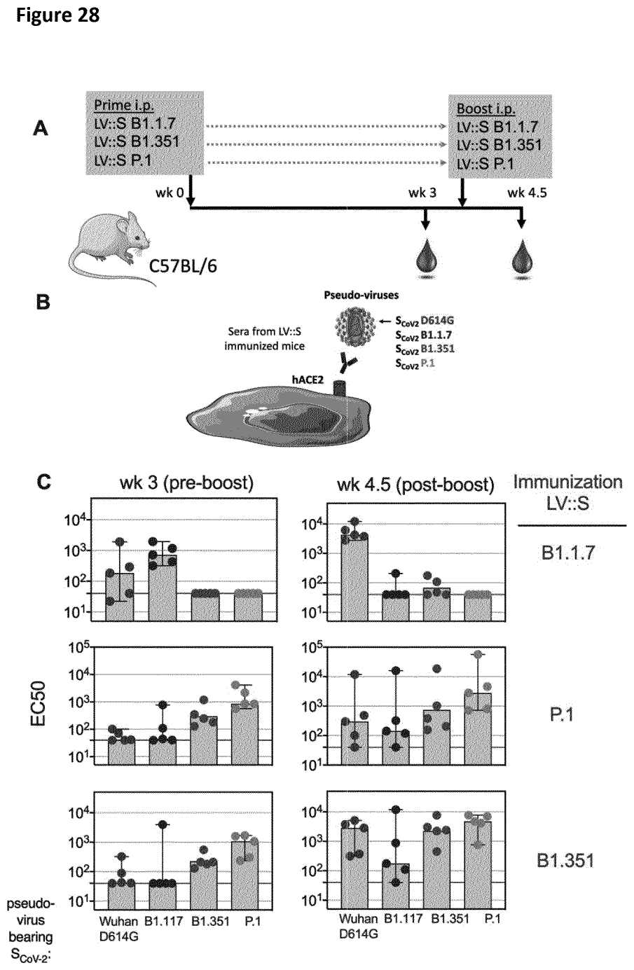

To identify the most cross-protective Spike variant, we primed and boosted C57BL/6 mice with LV encoding each Spike of interest (FIG. 28A), and assessed their cross-sero-neutralization potential by use of pseudo-viruses carrying each Spike (FIG. 28B). As shown in the FIG. 28C, we observed that:
(i) sera from mice immunized with LV::S_{CoV-2 B1.1.7} neutralized at high EC50 pseudo-viruses harboring S_{CoV-2 Wuhan} and LV::S_{CoV-2 B1.1.7}, but poorly pseudo-viruses harboring S_{CoV-2} B1.351 and LV::S_{CoV-2 P.1}.

(ii) sera from mice immunized with LV::S_{CoV-2 P.1} neutralized at high EC50 pseudo-viruses harboring S_{CoV-2 P.1} and LV::S_{CoV-2 B1.351}, but poorly pseudo-viruses harboring S_{CoV-2} Wuhan and LV::S_{CoV-2 B1.1.7}.

(iii) sera from mice immunized with LV::S_{CoV-2 B1.351} not only neutralized at high EC50 pseudo-viruses carrying S_{CoV-2 P.1} and LV::S_{CoV-2 B1.351} but also pseudo-viruses harboring S_{CoV-2} Wuhan and LV::S_{CoV-2 B1.1.7}.

These results designate the Spike sequence from the B1.351 (South African or β) variant as the most cross-reactive immunogen in terms of neutralizing antibodies.

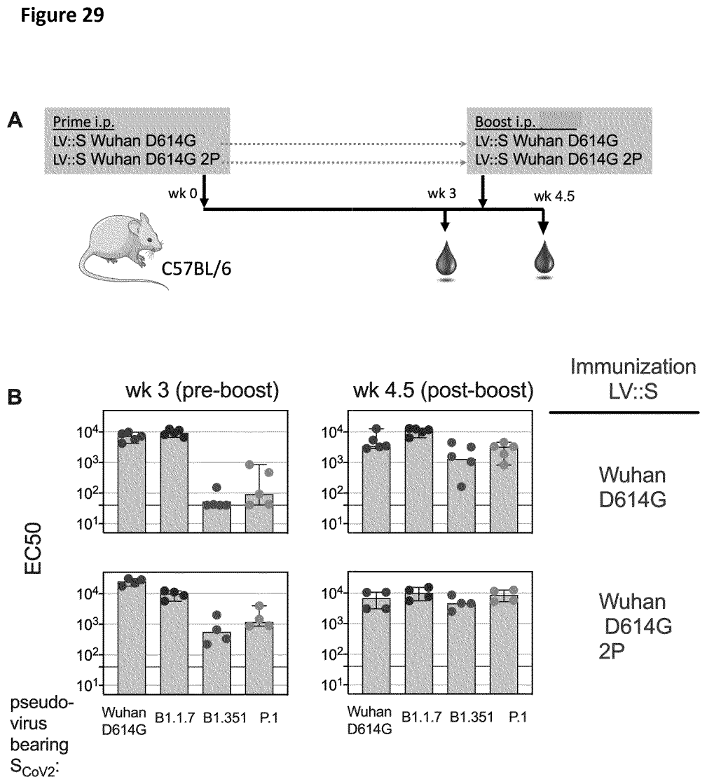

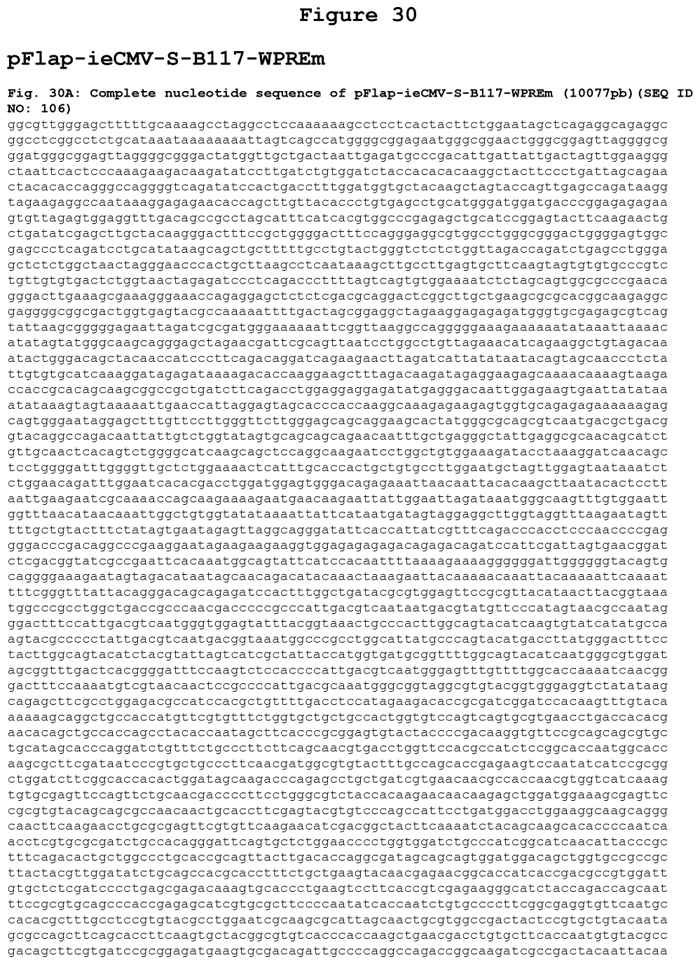

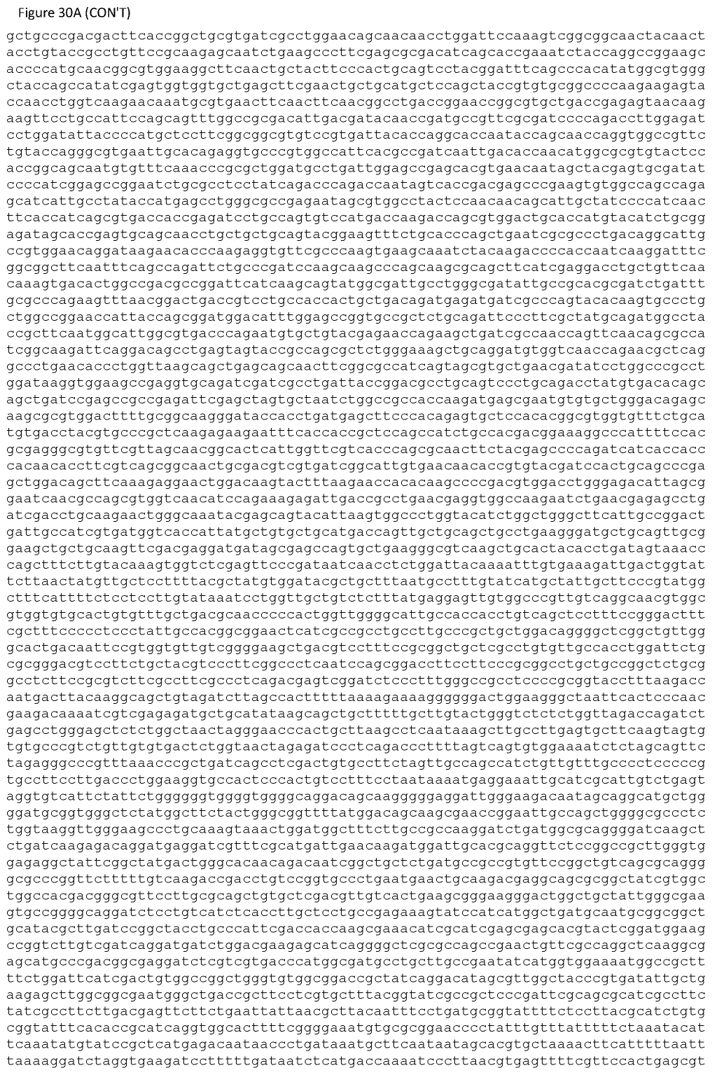

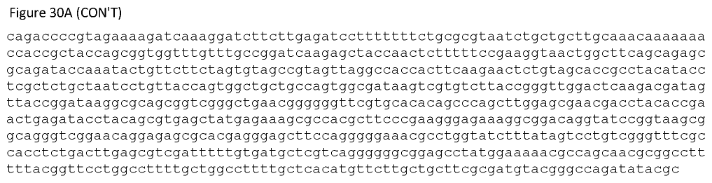

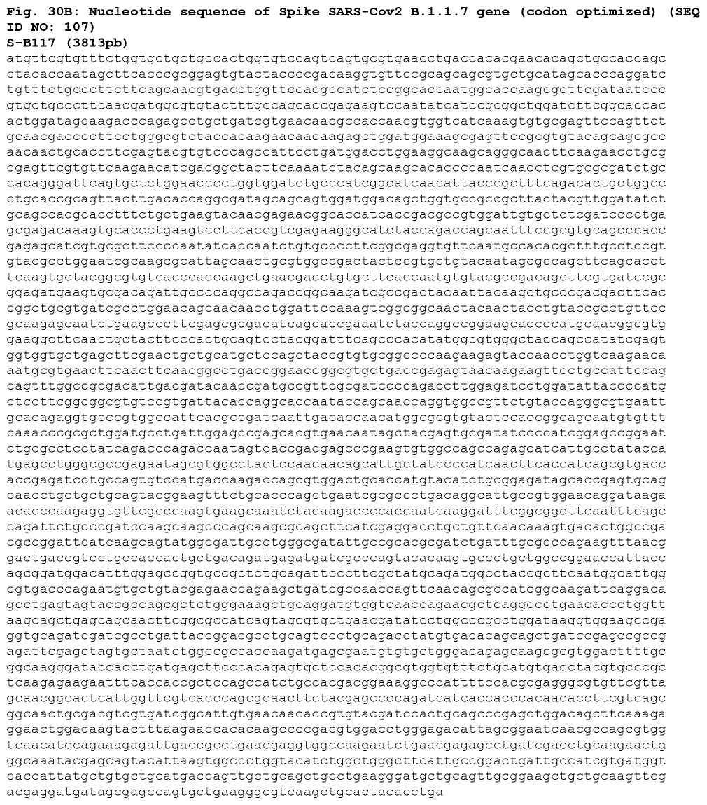

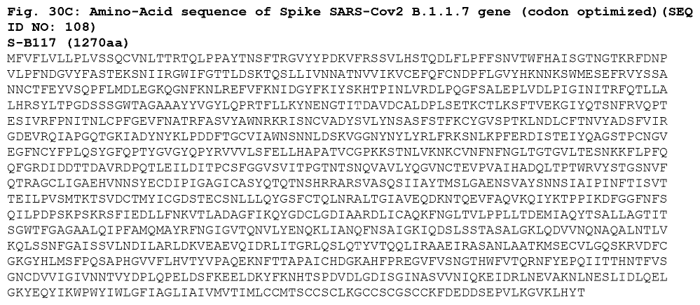

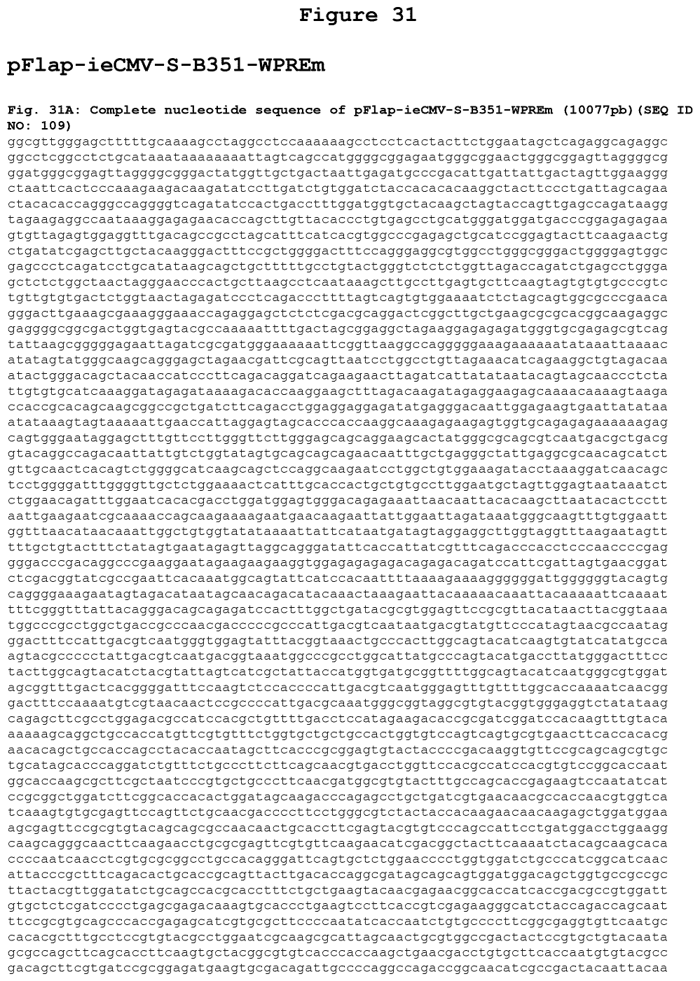

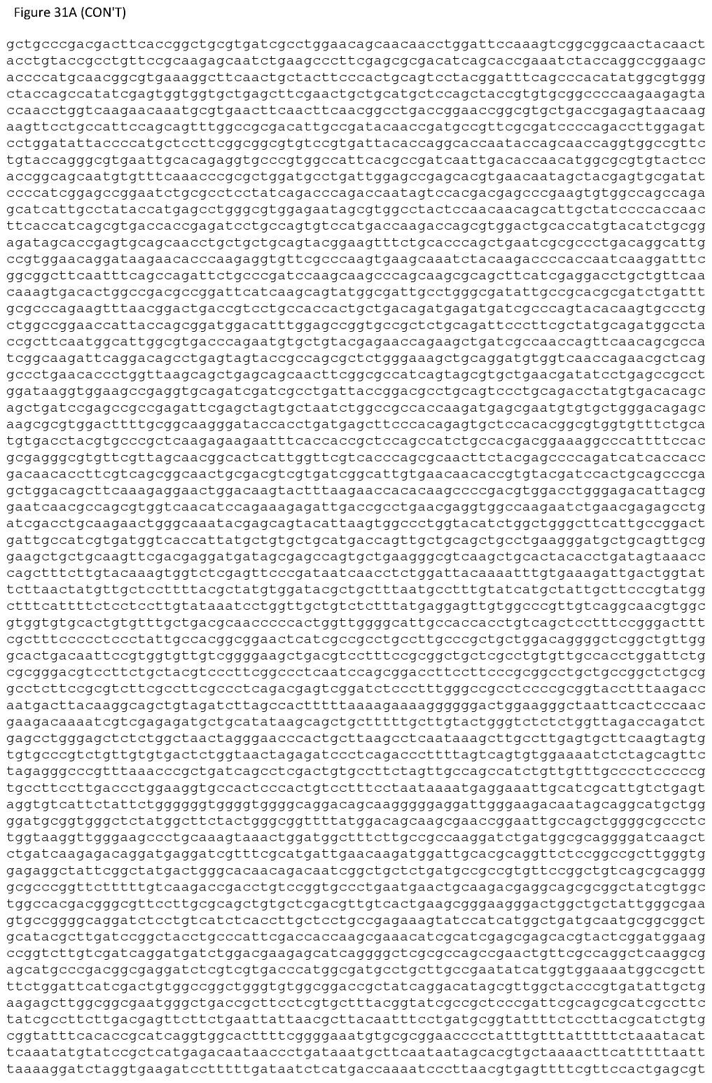

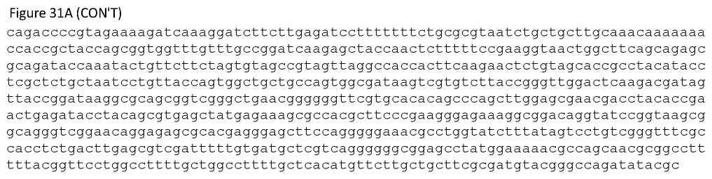

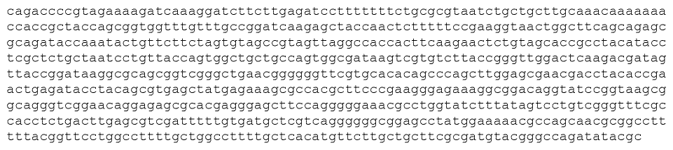

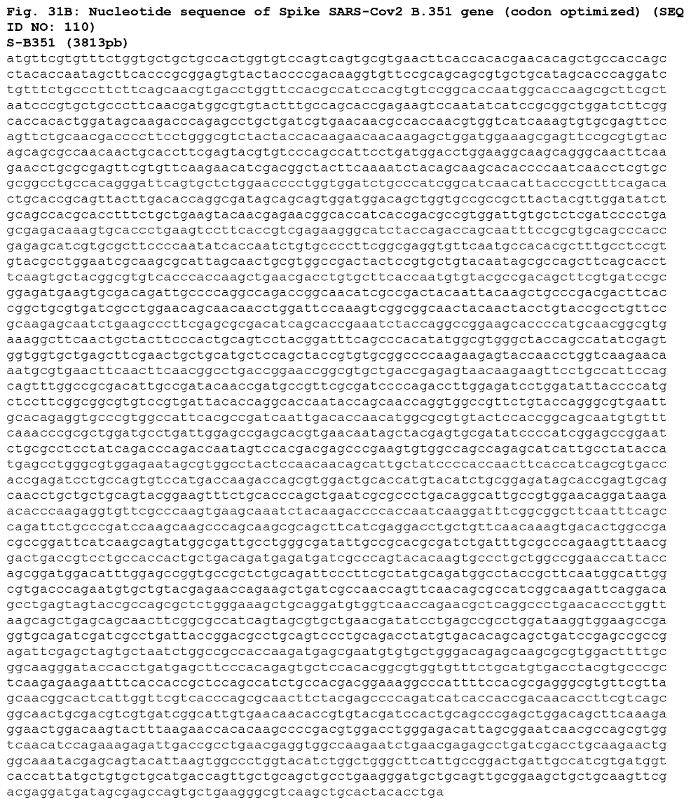

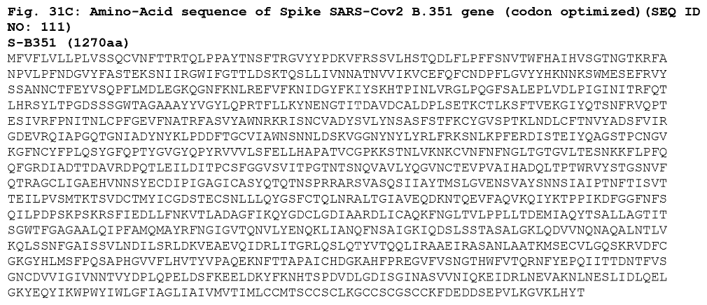

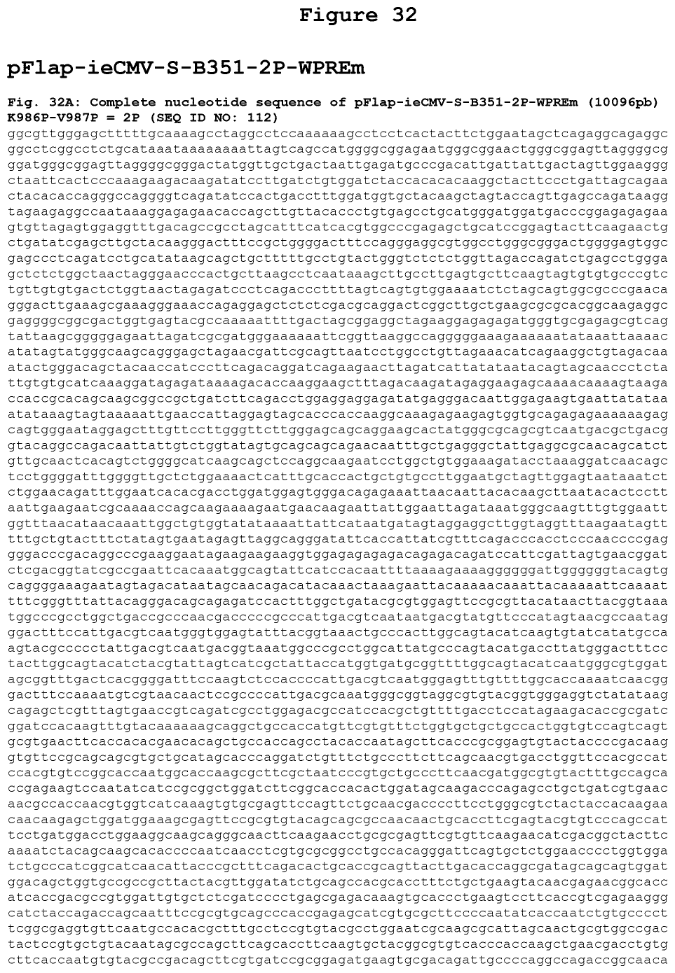

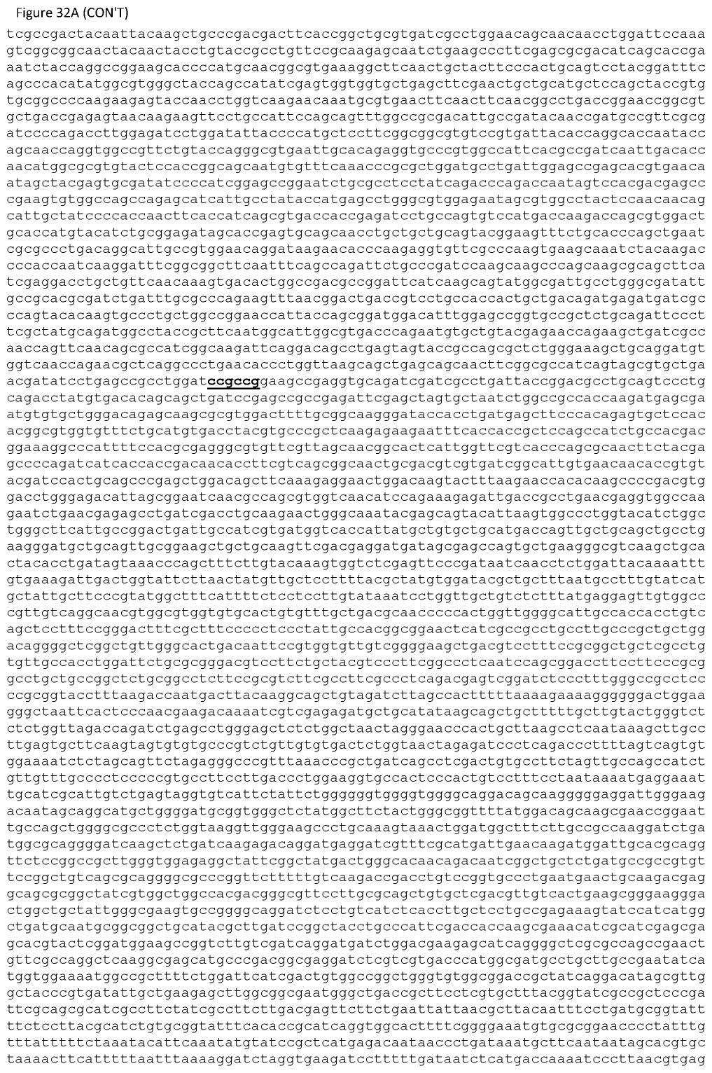

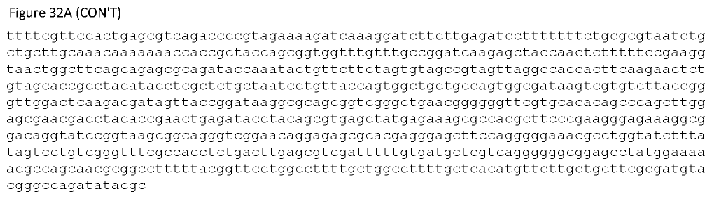

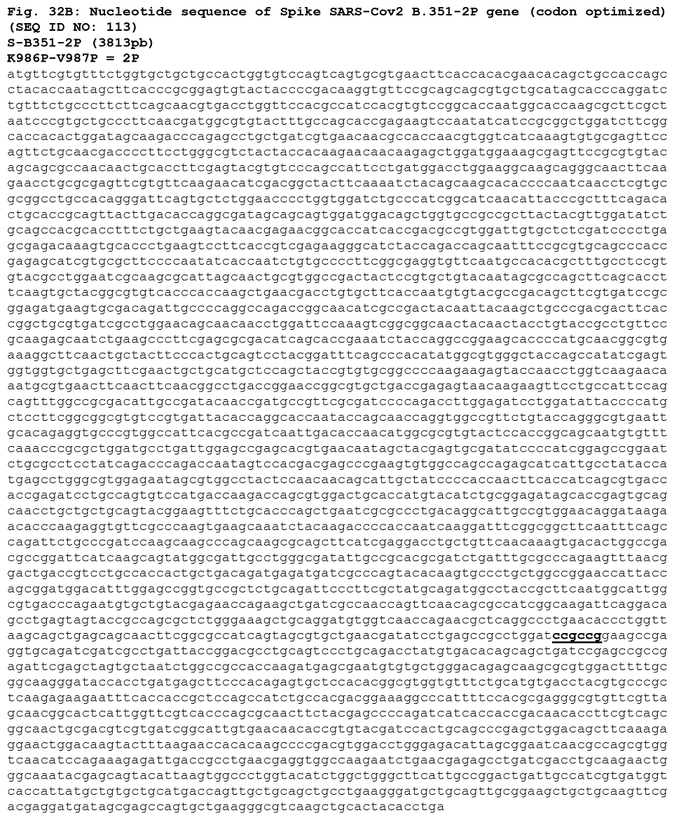

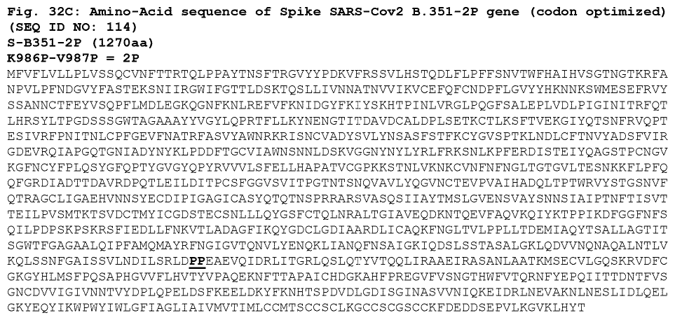

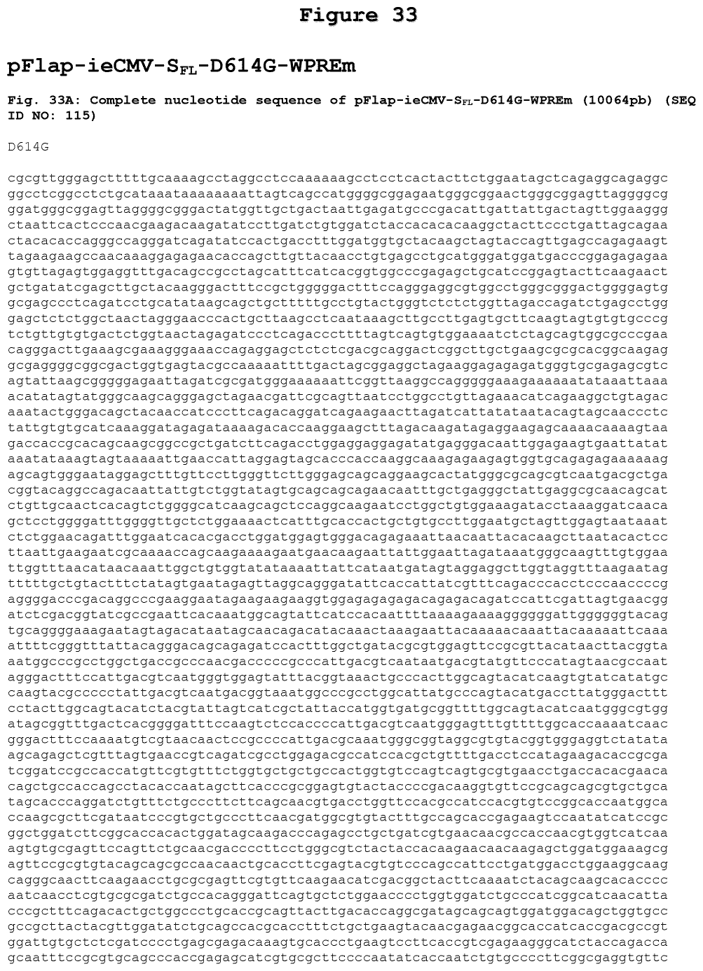

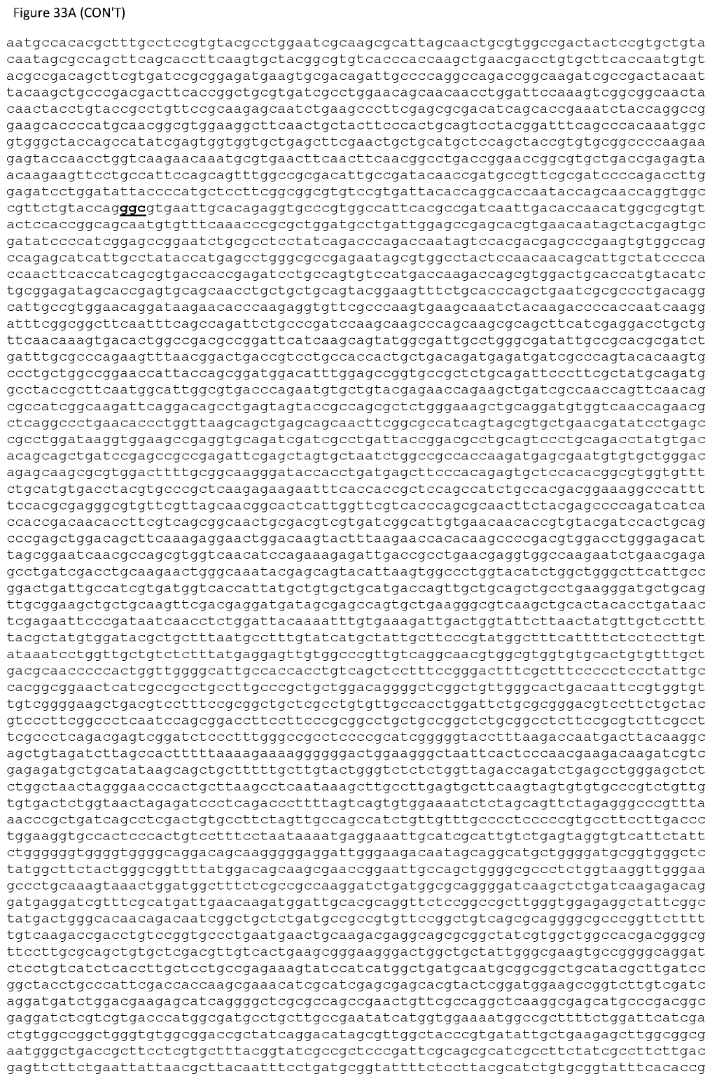

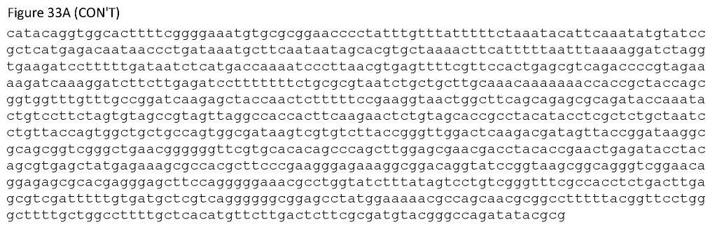

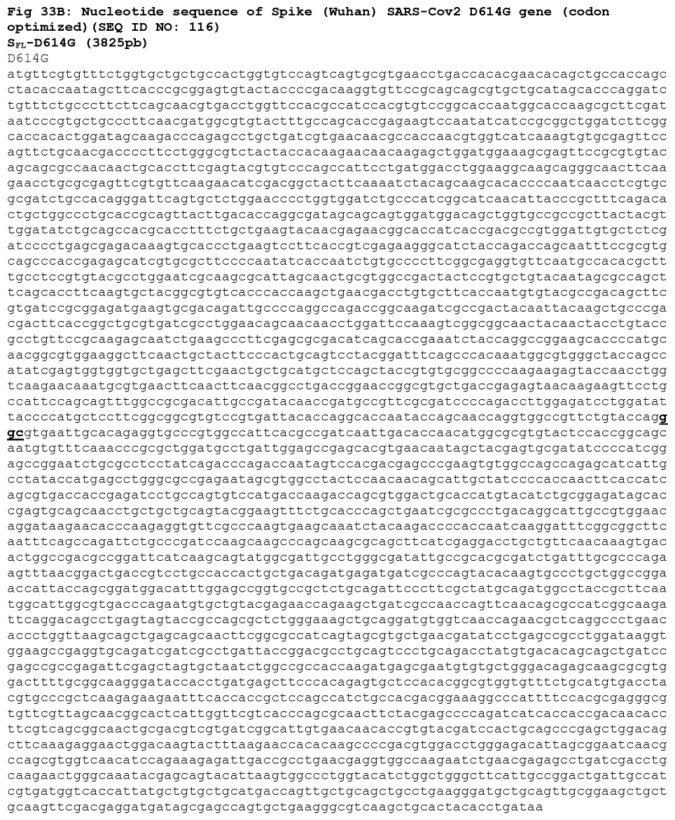

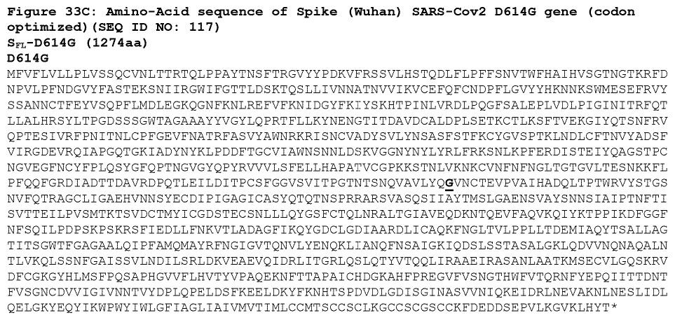

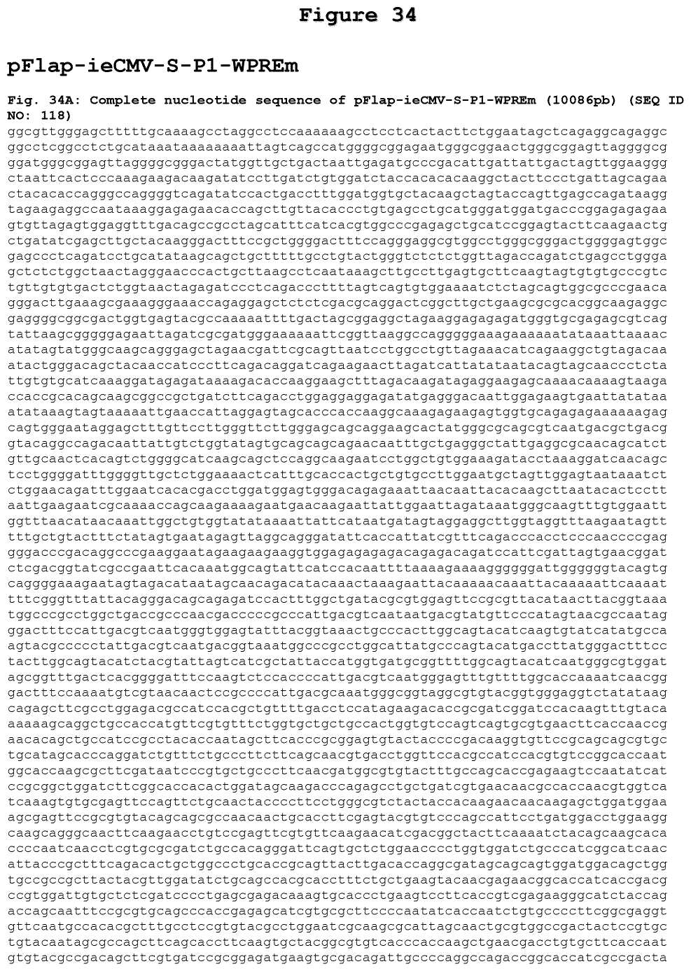

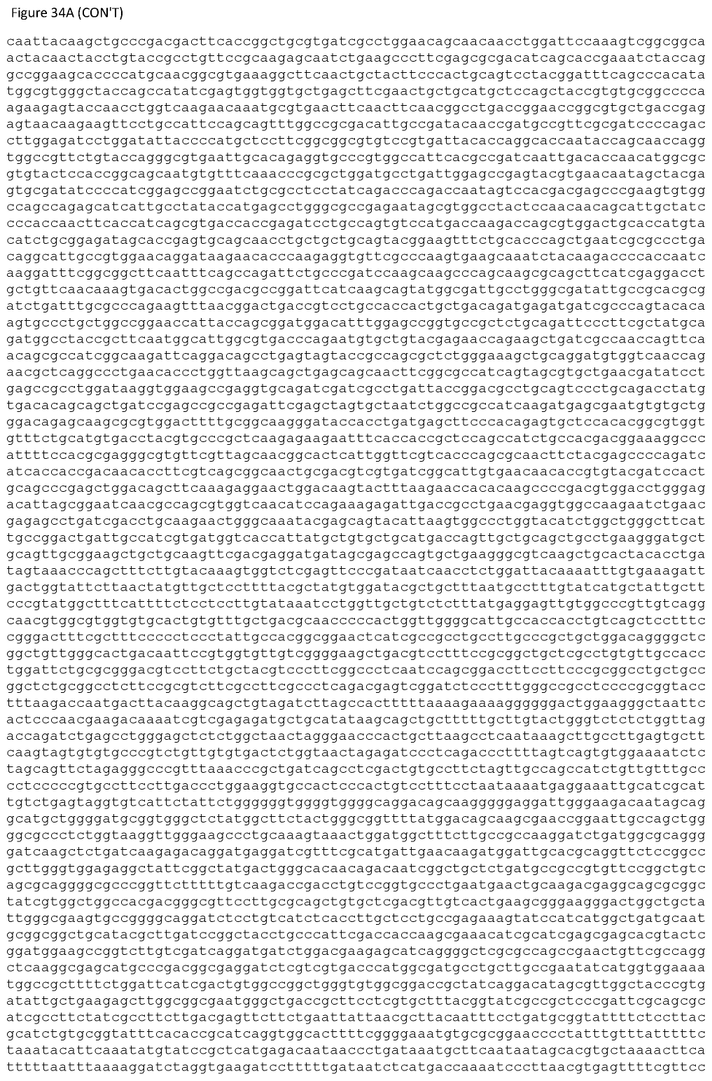

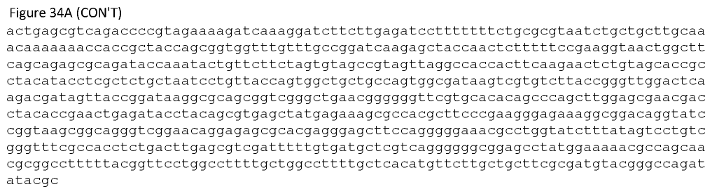

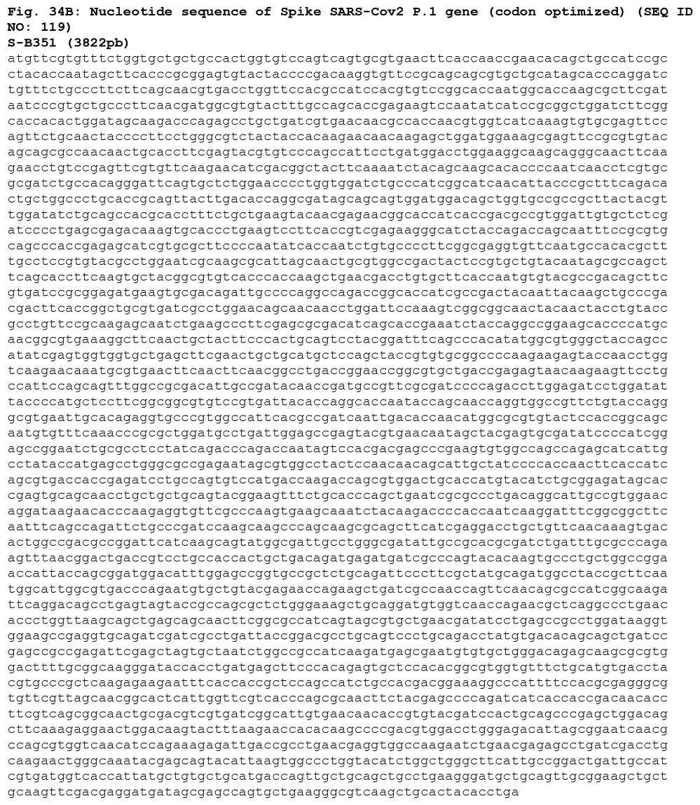

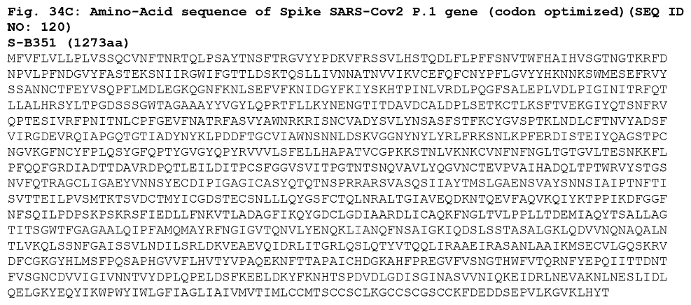

Furthermore, we showed that in the context of LV, Spike stabilization by K⁹⁸⁶P-V⁹⁸⁷P substitutions (2P) considerably improves the (cross) neutralizing antibody activity (FIG. 29A-C).

Therefore, our future lead antigen candidate is the full-length Spike from the B1.351 (South African or β) variant with 2P.

### REFERENCES CITED FOR EXAMPLE 1

Amanat, F., and F. Krammer. 2020. SARS-CoV-2 Vaccines: Status Report. Immunity 52:583-589.
Beignon, A. S., K. Mollier, C. Liard, F. Coutant, S. Munier, J. Riviere, P. Souque, and P. Charneau. 2009. Lentiviral vector-based prime/boost vaccination against AIDS: pilot study shows protection against Simian immunodeficiency virus SIVmac251 challenge in macaques. J Virol 83:10963-10974.
Belouzard, S., V. C. Chu, and G. R. Whittaker. 2009. Activation of the SARS coronavirus spike protein via sequential proteolytic cleavage at two distinct sites. Proc Natl Acad Sci USA 106:5871-5876.
Bourgine, M., S. Crabe, Y. Lobaina, G. Guillen, J. C. Aguilar, and M. L. Michel. 2018. Nasal route favors the induction of CD4(+) T cell responses in the liver of HBV-carrier mice immunized with a recombinant hepatitis B surface- and core-based therapeutic vaccine. Antiviral Res 153:23-32.
Bulla, V., D. R. Negri, P. Leone, M. Borghi, R. Bona, Z. Michelini, D. Compagnoni, C. Sgadari, B. Ensoli, and A. Cara. 2006. Evaluation of a self-inactivating lentiviral vector expressing simian immunodeficiency virus gag for induction of specific immune responses in vitro and in vivo. Viral Immunol 19:690-701.
Chandrashekar, A., J. Liu, A. J. Martinot, K. McMahan, N. B. Mercado, L. Peter, L. H. Tostanoski, J. Yu, Z. Maliga, M. Nekorchuk, K. Busman-Sahay, M. Terry, L. M. Wrijil, S. Ducat, D. R. Martinez, C. Atyeo, S. Fischinger, J. S. Burke, M. D. Slein, L. Pessaint, A. Van Ry, J. Greenhouse, T. Taylor, K. Blade, A. Cook, B. Finneyfrock, R. Brown, E. Teow, J. Velasco, R. Zahn, F. Wegmann, P. Abbink, E. A. Bondzie, G. Dagotto, M. S. Gebre, X. He, C. Jacob-Dolan, N. Kordana, Z. Li, M. A. Lifton, S. H. Mahrokhian, L. F. Maxfield, R. Nityanandam, J. P. Nkolola, A. G. Schmidt, A. D. Miller, R. S. Baric, G. Alter, P. K. Sorger, J. D. Estes, H. Andersen, M. G. Lewis, and D. H. Barouch. 2020. SARS-CoV-2 infection protects against rechallenge in rhesus macaques. Science, May 2020:eabc4776. doi: 10.1126/science.abc4776. Online ahead of print. PMID: 32434946.
Corman, V., T. Bleicker, S. Brünink, and C. Drosten. 2020. Diagnostic detection of 2019-nCoV by real-time RT-PCR. https://www.who.int/docs/default-source/coronaviruse/protocol-v2-1.pdf
Cousin, C., M. Oberkampf, T. Felix, P. Rosenbaum, R. Weil, S. Fabrega, V. Morante, D. Negri, A. Cara, G. Dadaglio, and C. Leclerc. 2019. Persistence of Integrase-Deficient Lentiviral Vectors Correlates with the Induction of STING-Independent CD8(+) T Cell Responses. Cell Rep 26:1242-1257 e1247.
Coutant, F., R. Y. Sanchez David, T. Felix, A. Boulay, L. Caleechurn, P. Souque, C. Thouvenot, C. Bourgouin, A. S. Beignon, and P. Charneau. 2012. A nonintegrative lentiviral vector-based vaccine provides long-term sterile protection against malaria. PLoS One 7:e48644.
Coutard, B., C. Valle, X. de Lamballerie, B. Canard, N. G. Seidah, and E. Decroly. 2020. The spike glycoprotein of the new coronavirus 2019-nCoV contains a furin-like cleavage site absent in CoV of the same clade. Antiviral Res 176:104742.
Di Nunzio, F., T. Felix, N. J. Arhel, S. Nisole, P. Charneau, and A. S. Beignon. 2012. HIV-derived vectors for therapy and vaccination against HIV. Vaccine 30:2499-2509.
Esslinger, C., P. Romero, and H. R. MacDonald. 2002. Efficient transduction of dendritic cells and induction of a T-cell response by third-generation lentivectors. Hum Gene Ther 13:1091-1100.
Gallinaro, A., M. Borghi, R. Bona, F. Grasso, L. Calzoletti, L. Palladino, S. Cecchetti, M. F. Vescio, D. Macchia, V. Morante, A. Canitano, N. Temperton, M. R. Castrucci, M. Salvatore, Z. Michelini, A. Cara, and D. Negri. 2018. Integrase Defective Lentiviral Vector as a Vaccine Platform for Delivering Influenza Antigens. Front Immunol 9:171.
Gao, Q., L. Bao, H. Mao, L. Wang, K. Xu, M. Yang, Y. Li, L. Zhu, N. Wang, Z. Lv, H. Gao, X. Ge, B. Kan, Y. Hu, J. Liu, F. Cai, D. Jiang, Y. Yin, C. Qin, J. Li, X. Gong, X. Lou, W. Shi, D. Wu, H. Zhang, L. Zhu, W. Deng, Y. Li, J. Lu, C. Li, X. Wang, W. Yin, Y. Zhang, and C. Qin. 2020. Rapid development of an inactivated vaccine candidate for SARS-CoV-2. Science 2020 Jul. 3; 369(6499):77-81. doi: 10.1126/science.abc1932. Epub 2020 May 6.PMID: 32376603.
Guo, Y. R., Q. D. Cao, Z. S. Hong, Y. Y. Tan, S. D. Chen, H. J. Jin, K. S. Tan, D. Y. Wang, and Y. Yan. 2020. The origin, transmission and clinical therapies on coronavirus disease 2019 (COVID-19) outbreak—an update on the status. Mil Med Res 7:11.
He, Y., J. Zhang, Z. Mi, P. Robbins, and L. D. Falo, Jr. 2005. Immunization with lentiviral vector-transduced dendritic cells induces strong and long-lasting T cell responses and therapeutic immunity. J Immunol 174:3808-3817.
Hu, B., A. Tai, and P. Wang. 2011. Immunization delivered by lentiviral vectors for cancer and infectious diseases. Immunol Rev 239:45-61.
Iglesias, M. C., M. P. Frenkiel, K. Mollier, P. Souque, P. Despres, and P. Charneau. 2006. A single immunization with a minute dose of a lentiviral vector-based vaccine is highly effective at eliciting protective humoral immunity against West Nile virus. J Gene Med 8:265-274.
Ku, M. W., F. Anna, F. Souque, S. Petres, M. Prot, E. Simon-Loriere, P. Charneau, and M. Bourgine. 2020. A Single Dose of NILV-Based Vaccine Provides Rapid and Durable Protection against Zika Virus. Mol Ther 2020 May 20; 51525-0016(20)30250-1. doi: 10.1016/j.ymthe.2020.05.016.
Ku, M. W., P. Authié, P. Souque, M. Bourgine, M. Romano, P. Charneau, and L. Majlessi. Submitted. High-Quality Memory T Cells by Programmed Antigen Expression in Dendritic Cells Induced by Lentiviral Vector. (In revision)
Lai, A. L., J. K. Millet, S. Daniel, J. H. Freed, and G. R. Whittaker. 2017. The SARS-CoV Fusion Peptide Forms an Extended Bipartite Fusion Platform that Perturbs Membrane Order in a Calcium-Dependent Manner. J Mol Biol 429:3875-3892.
Lorin, V., and H. Mouquet. 2015. Efficient generation of human IgA monoclonal antibodies. J Immunol Methods 422:102-110.
Qiu, H., S. Sun, H. Xiao, J. Feng, Y. Guo, W. Tai, Y. Wang, L. Du, G. Zhao, and Y. Zhou. 2016. Single-dose treatment with a humanized neutralizing antibody affords full protection of a human transgenic mouse model from lethal Middle East respiratory syndrome (MERS)-coronavirus infection. Antiviral Res 132:141-148.
Rosenberg, S. A., Y. Zhai, J. C. Yang, D. J. Schwartzentruber, P. Hwu, F. M. Marincola, S. L. Topalian, N. P. Restifo, C. A. Seipp, J. H. Einhorn, B. Roberts, and D. E. White. 1998. Immunizing patients with metastatic melanoma using recombinant adenoviruses encoding MART-1 or gp100 melanoma antigens. J Natl Cancer Inst 90:1894-1900.
Schirmbeck, R., J. Reimann, S. Kochanek, and F. Kreppel. 2008. The immunogenicity of adenovirus vectors limits the multispecificity of CD8 T-cell responses to vector-encoded transgenic antigens. Mol Ther 16:1609-1616.
Sia, S. F., L. M. Yan, A. W. H. Chin, K. Fung, K. T. Choy, A. Y. L. Wong, P. Kaewpreedee, R. Perera, L. L. M. Poon, J. M. Nicholls, M. Peiris, and H. L. Yen. 2020. Pathogenesis and transmission of SARS-CoV-2 in golden hamsters. Nature 2020 May 14. doi: 10.1038/s41586-020-2342-5. Online ahead of print.PMID: 32408338.
Sterlin, D., A. Mathian, M. Miyara, A. Mohr, F. Anna, L. Claër, P. Quentric, J. Fadlallah, P. Ghillani, C. Gunn, R. Hockett, S. Mudumba, A. Guihot, C. Luyt, J. Mayaux, A. Beurton, S. Fourati, J. Lacorte, H. Yssel, C. Parizot, K. Dorgham, P. Charneau, Z. Amoura, and G. Gorochov. IgA dominates the early neutralizing antibody response to SARS-CoV-2. (in preparation).
Vabret, N., G. J. Britton, C. Gruber, S. Hegde, J. Kim, M. Kuksin, R. Levantovsky, L. Malle, A. Moreira, M. D. Park, L. Pia, E. Risson, M. Saffern, B. Salome, M. Esai Selvan, M. P. Spindler, J. Tan, V. van der Heide, J. K. Gregory, K. Alexandropoulos, N. Bhardwaj, B. D. Brown, B. Greenbaum, Z. H. Gumus, D. Homann, A. Horowitz, A. O. Kamphorst, M. A. Curotto de Lafaille, S. Mehandru, M. Merad, R. M. Samstein, and P. Sinai Immunology Review. 2020. Immunology of COVID-19: Current State of the Science. Immunity 52:910-941.
Walls A. C., Y. J. Park, M. A. Tortorici, A. Wall, A. T. McGuire, and D. Veesler. 2020. Structure, Function, and Antigenicity of the SARS-CoV-2 Spike Glycoprotein. Cell 181:281-292 e286.
Wan, Y., J. Shang, S. Sun, W. Tai, J. Chen, Q. Geng, L. He, Y. Chen, J. Wu, Z. Shi, Y. Zhou, L. Du, and F. Li. 2020. Molecular Mechanism for Antibody-Dependent Enhancement of Coronavirus Entry. J Virol 94:
Wang, Q., Y. Qiu, J. Y. Li, Z. J. Zhou, C. H. Liao, and X. Y. Ge. 2020. A Unique Protease Cleavage Site Predicted in the Spike Protein of the Novel Pneumonia Coronavirus (2019-nCoV) Potentially Related to Viral Transmissibility. Virol Sin 2020 June; 35(3):337-339. doi: 10.1007/s12250-020-00212-7. Epub 2020 Mar. 20.
Yu, J., L. H. Tostanoski, L. Peter, N. B. Mercado, K. McMahan, S. H. Mahrokhian, J. P. Nkolola, J. Liu, Z. Li, A. Chandrashekar, D. R. Martinez, C. Loos, C. Atyeo, S. Fischinger, J. S. Burke, M. D. Slein, Y. Chen, A. Zuiani, N. L. FJ, M. Travers, S. Habibi, L. Pessaint, A. Van Ry, K. Blade, R. Brown, A. Cook, B. Finneyfrock, A. Dodson, E. Teow, J. Velasco, R. Zahn, F. Wegmann, E. A. Bondzie, G. Dagotto, M. S. Gebre, X. He, C. Jacob-Dolan, M. Kirilova, N. Kordana, Z. Lin, L. F. Maxfield, F. Nampanya, R. Nityanandam, J. D. Ventura, H. Wan, Y. Cai, B. Chen, A. G. Schmidt, D. R. Wesemann, R. S. Baric, G. Alter, H. Andersen, M. G. Lewis, and D. H. Barouch. 2020. DNA vaccine protection against SARS-CoV-2 in rhesus macaques. Science 2020 May 20; eabc6284. doi: 10.1126/science.abc6284. PMID: 32434945.
Zennou, V., C. Petit, D. Guetard, U. Nerhbass, L. Montagnier, and P. Charneau. 2000. HIV-1 genome nuclear import is mediated by a central DNA flap. Cell 101:173-185.
Zhao, J., K. Li, C. Wohlford-Lenane, S. S. Agnihothram, C. Fett, J. Zhao, M. J. Gale, Jr., R. S. Baric, L. Enjuanes, T. Gallagher, P. B. McCray, Jr., and S. Perlman. 2014. Rapid generation of a mouse model for Middle East respiratory syndrome. Proc Natl Acad Sci USA 111:4970-4975.
Zhu, F. C., Y. H. Li, X. H. Guan, L. H. Hou, W. J. Wang, J. X. Li, S. P. Wu, B. S. Wang, Z. Wang, L. Wang, S. Y. Jia, H. D. Jiang, L. Wang, T. Jiang, Y. Hu, J. B. Gou, S. B. Xu, J. J. Xu, X. W. Wang, W. Wang, and W. Chen. 2020. Safety, tolerability, and immunogenicity of a recombinant adenovirus type-5 vectored COVID-19 vaccine: a dose-escalation, open-label, non-randomised, first-in-human trial. Lancet. 2020 Jun. 13; 395(10240):1845-1854. doi: 10.1016/S0140-6736(20)31208-3. Epub 2020 May 22. P.

### REFERENCES CITED FOR EXAMPLES 2 AND 3

Aghagoli, G., Gallo Marin, B., Katchur, N. J., Chaves-Sell, F., Asaad, W. F., and Murphy, S. A. (2020). Neurological Involvement in COVID-19 and Potential Mechanisms: A Review. Neurocrit Care.
Bergmann, C. C., T. E. Lane, and S. A. Stohlman. 2006. Coronavirus infection of the central nervous system: host-virus stand-off. Nat Rev Microbiol 4:121-132.
Anna, F., Goyard, S., Lalanne, A. I., Nevo, F., Gransagne, M., Souque, P., Louis, D., Gillon, V., Turbiez, I., Bidard, F. C., et al. (2020). High seroprevalence but short-lived immune response to SARS-CoV-2 infection in Paris. Eur J Immunol.
Bos, R., Rutten, L., van der Lubbe, J. E. M., Bakkers, M. J. G., Hardenberg, G., Wegmann, F., Zuijdgeest, D., de Wilde, A. H., Koornneef, A., Verwilligen, A., et al. (2020). Ad26 vector-based COVID-19 vaccine encoding a prefusion-stabilized SARS-CoV-2 Spike immunogen induces potent humoral and cellular immune responses. NPJ Vaccines 5, 91
Bourgonje, A. R., Abdulle, A. E., Timens, W., Hillebrands, J. L., Navis, G. J., Gordijn, S. J., Bolling, M. C., Dijkstra, G., Voors, A. A., Osterhaus, A. D., et al. (2020). Angiotensin-converting enzyme 2 (ACE2), SARS-CoV-2 and the pathophysiology of coronavirus disease 2019 (COVID-19). J Pathol 251, 228-248.
Chandrashekar, A., Liu, J., Martinot, A. J., McMahan, K., Mercado, N. B., Peter, L., Tostanoski, L. H., Yu, J., Maliga, Z., Nekorchuk, M., et al. (2020). SARS-CoV-2 infection protects against rechallenge in rhesus macaques. Science May 2020:eabc4776 doi: 101126/scienceabc4776 PMID: 32434946.
Chen, R., Wang, K., Yu, J., Howard, D., French, L., Chen, Z., Wen, C., and Xu, Z. (2020). The spatial and cell-type distribution of SARS-CoV-2 receptor ACE2 in human and mouse brain. BioRxiv.
Chow, Y. H., O'Brodovich, H., Plumb, J., Wen, Y., Sohn, K. J., Lu, Z., Zhang, F., Lukacs, G. L., Tanswell, A. K., Hui, C. C., et al. (1997). Development of an epithelium-specific expression cassette with human DNA regulatory elements for transgene expression in lung airways. Proc Natl Acad Sci USA 94, 14695-14700.
Corman, V., Bleicker, T., Brünink, S., and Drosten, C. (2020). Diagnostic detection of 2019-nCoV by real-time RT-PCR. https://wwwwhoint/docs/default-sou rce/coronavi ruse/protocol-v2-1pdf.
Cupovic, J., Onder, L., Gil-Cruz, C., Weiler, E., Caviezel-Firner, S., Perez-Shibayama, C., Rulicke, T., Bechmann, I., and Ludewig, B. (2016). Central Nervous System Stromal Cells Control Local CD8(+) T Cell Responses during Virus-Induced Neuroinflammation. Immunity 44, 622-633.
Desforges, M., Le Coupanec, A., Stodola, J. K., Meessen-Pinard, M., and Talbot, P. J. (2014). Human coronaviruses: viral and cellular factors involved in neuroinvasiveness and neuropathogenesis. Virus Res 194, 145-158.
Di Nunzio, F., Felix, T., Arhel, N. J., Nisole, S., Charneau, P., and Beignon, A. S. (2012). HIV-derived vectors for therapy and vaccination against HIV. Vaccine 30, 2499-2509.
Dogan, R. I., Getoor, L., Wilbur, W. J., and Mount, S. M. (2007). Features generated for computational splice-site prediction correspond to functional elements. BMC Bioinformatics 8, 410.
Firat H. et al. The Journal of Gene Medicine 2002; 4: 38-45
Fotuhi, M., Mian, A., Meysami, S., and Raji, C. A. (2020). Neurobiology of COVID-19. J Alzheimers Dis 76, 3-19.
Glass, W. G., Subbarao, K., Murphy, B., and Murphy, P. M. (2004). Mechanisms of host defense following severe acute respiratory syndrome-coronavirus (SARS-CoV) pulmonary infection of mice. J Immunol 173, 4030-4039.
Guo, Y. R., Cao, Q. D., Hong, Z. S., Tan, Y. Y., Chen, S. D., Jin, H. J., Tan, K. S., Wang, D. Y., and Yan, Y. (2020). The origin, transmission and clinical therapies on coronavirus disease 2019 (COVID-19) outbreak—an update on the status. Mil Med Res 7, 11
Hsieh, C. L., Goldsmith, J. A., Schaub, J. M., DiVenere, A. M., Kuo, H. C., Javanmardi, K., Le, K. C., Wrapp, D., Lee, A. G., Liu, Y., et al. (2020). Structure-based design of prefusion-stabilized SARS-CoV-2 spikes. Science 369, 1501-1505.
Hu, B., Tai, A., and Wang, P. (2011). Immunization delivered by lentiviral vectors for cancer and infectious diseases. Immunol Rev 239, 45-61.
Hu, J., Jolkkonen, J., and Zhao, C. (2020). Neurotropism of SARS-CoV-2 and its neuropathological alterations: Similarities with other coronaviruses. Neurosci Biobehav Rev 119, 184-193.
Hoffmann, M., H. Kleine-Weber, S. Schroeder, N. Kruger, T. Herrler, S. Erichsen, T. S. Schiergens, G. Herrler, N. H. Wu, A. Nitsche, M. A. Muller, C. Drosten, and S. Pohlmann. 2020. SARS-CoV-2 Cell Entry Depends on ACE2 and TMPRSS2 and Is Blocked by a Clinically Proven Protease Inhibitor. Cell 181:271-280 e278
Jiang, R. D., Liu, M. Q., Chen, Y., Shan, C., Zhou, Y. W., Shen, X. R., Li, Q., Zhang, L., Zhu, Y., Si, H. R., et al. (2020). Pathogenesis of SARS-CoV-2 in Transgenic Mice Expressing Human Angiotensin-Converting Enzyme 2. Cell 182, 50-58 e58.
Koehler, D. R., Chow, Y. H., Plumb, J., Wen, Y., Rafii, B., Belcastro, R., Haardt, M., Lukacs, G. L., Post, M., Tanswell, A. K., et al. (2000). A human epithelium-specific vector optimized in rat pneumocytes for lung gene therapy. Pediatr Res 48, 184-190.
Ku, M. W., Anna, F., Souque, F., Petres, S., Prot, M., Simon-Loriere, E., Charneau, P., and Bourgine, M. (2020). A Single Dose of NILV-Based Vaccine Provides Rapid and Durable Protection against Zika Virus. Mol Ther 2020 May 20; 51525-0016(20)30250-1 doi: 101016/jymthe202005016.
Ku, M. W., Bourgine, M., Authié, P., Lopez, J., Nemirov, N., Moncoq, F., Noirat, A., Vesin, B., Nevo, F., Blanc, C., et al. (2021). Intranasal Vaccination with a Lentiviral Vector Protects against SARS-CoV-2 in Preclinical Animal Models
Cell Host and Microbe in press. PMID: 33357418
Lescure, F. X., Bouadma, L., Nguyen, D., Parisey, M., Wicky, P. H., Behillil, S., Gaymard, A., Bouscambert-Duchamp, M., Donati, F., Le Hingrat, Q., et al. (2020). Clinical and virological data of the first cases of COVID-19 in Europe: a case series. Lancet Infect Dis 20, 697-706.
Li, K., Wohlford-Lenane, C., Perlman, S., Zhao, J., Jewell, A. K., Reznikov, L. R., Gibson-Corley, K. N., Meyerholz, D. K., and McCray, P. B., Jr. (2016). Middle East Respiratory Syndrome Coronavirus Causes Multiple Organ Damage and Lethal Disease in Mice Transgenic for Human Dipeptidyl Peptidase 4. J Infect Dis 213, 712-722.
Liu, J., Li, S., Liu, J., Liang, B., Wang, X., Wang, H., Li, W., Tong, Q., Yi, J., Zhao, L., et al. (2020). Longitudinal characteristics of lymphocyte responses and cytokine profiles in the peripheral blood of SARS-CoV-2 infected patients. EBioMedicine 55, 102763.
Lopez, J., Anna, F., Authié, P., Pawlik, A., Ku, M. W., Blanc, C., Souque, P., Moncoq, F., Noirat, A., Sougakoff, W., et al. (in preparation). An Optimized Poly-antigenic Lentiviral Vector Induces Protective CD4+ T-Cell Immunity and Predicts a Booster Vaccine against Mycobacterium tuberculosis.
Mao, L., Jin, H., Wang, M., Hu, Y., Chen, S., He, Q., Chang, J., Hong, C., Zhou, Y., Wang, D., et al. (2020). Neurologic Manifestations of Hospitalized Patients With Coronavirus Disease 2019 in Wuhan, China. JAMA Neurol 77, 683-690.
McCallum, M., Walls, A. C., Bowen, J. E., Corti, D., and Veesler, D. (2020). Structure-guided covalent stabilization of coronavirus spike glycoprotein trimers in the closed conformation. Nat Struct Mol Biol 27, 942-949.
McCray, P. B., Jr., Pewe, L., Wohlford-Lenane, C., Hickey, M., Manzel, L., Shi, L., Netland, J., Jia, H. P., Halabi, C., Sigmund, C. D., et al. (2007). Lethal infection of K18-hACE2 mice infected with severe acute respiratory syndrome coronavirus. J Virol 81, 813-821.
Meinhardt, J., Radke, J., Dittmayer, C., Franz, J., Thomas, C., Mothes, R., Laue, M., Schneider, J., Brunink, S., Greuel, S., et al. (2020). Olfactory transmucosal SARS-CoV-2 invasion as a port of central nervous system entry in individuals with COVID-19. Nat Neurosci.
Menachery, V. D., Yount, B. L., Jr., Sims, A. C., Debbink, K., Agnihothram, S. S., Gralinski, L. E., Graham, R. L., Scobey, T., Plante, J. A., Royal, S. R., et al. (2016). SARS-like WIV1-CoV poised for human emergence. Proc Natl Acad Sci USA 113, 3048-3053.
Munoz-Fontela, C., Dowling, W. E., Funnell, S. G. P., Gsell, P. S., Riveros-Balta, A. X., Albrecht, R. A., Andersen, H., Baric, R. S., Carroll, M. W., Cavaleri, M., et al. (2020). Animal models for COVID-19. Nature 586, 509-515.
Nakagawa, T., and Hoogenraad, C. C. (2011). Lentiviral transgenesis. Methods Mol Biol 693, 117-142.
Netland, J., Meyerholz, D. K., Moore, S., Cassell, M., and Perlman, S. (2008). Severe acute respiratory syndrome coronavirus infection causes neuronal death in the absence of encephalitis in mice transgenic for human ACE2. J Virol 82, 7264-7275.
Park, F. 2007. Lentiviral vectors: are they the future of animal transgenesis? Physiol Genomics 31:159-173
L. S., Salsano, E., and Grimaldi, M. (2020). Magnetic Resonance Imaging Alteration of the Brain in a Patient With Coronavirus Disease 2019 (COVID-19) and Anosmia. JAMA Neurol 77, 1028-1029.
Roman, G. C., Spencer, P. S., Reis, J., Buguet, A., Faris, M. E. A., Katrak, S. M., Lainez, M., Medina, M. T., Meshram, C., Mizusawa, H., et al. (2020). The neurology of COVID-19 revisited: A proposal from the Environmental Neurology Specialty Group of the World Federation of Neurology to implement international neurological registries. J Neurol Sci 414, 116884.
Rosenberg, S. A., Zhai, Y., Yang, J. C., Schwartzentruber, D. J., Hwu, P., Marincola, F. M., Topalian, S. L., Restifo, N. P., Seipp, C. A., Einhorn, J. H., et al. (1998). Immunizing patients with metastatic melanoma using recombinant adenoviruses encoding MART-1 or gp100 melanoma antigens. J Natl Cancer Inst 90, 1894-1900.
Sayes, F., C. Blanc, L. S. Ates, N. Deboosere, M. Orgeur, F. Le Chevalier, M. I. Groschel, W. Frigui, O. R. Song, R. Lo-Man, F. Brossier, W. Sougakoff, D. Bottai, P. Brodin, P. Charneau, R. Brosch, and L. Majlessi. 2018. Multiplexed Quantitation of Intraphagocyte Mycobacterium tuberculosis Secreted Protein Effectors. Cell Rep 23:1072-1084
Schirmbeck, R., Reimann, J., Kochanek, S., and Kreppel, F. (2008). The immunogenicity of adenovirus vectors limits the multispecificity of CD8 T-cell responses to vector-encoded transgenic antigens. Mol Ther 16, 1609-1616.
Sia, S. F., Yan, L. M., Chin, A. W. H., Fung, K., Choy, K. T., Wong, A. Y. L., Kaewpreedee, P., Perera, R., Poon, L. L. M., Nicholls, J. M., et al. (2020). Pathogenesis and transmission of SARS-CoV-2 in golden hamsters. Nature 2020 May 14 doi: 101038/s41586-020-2342-5 Online ahead of printPMID: 32408338.
Song, E., Zhang, C., lsraelow, B., Lu-Culligan, A., Prado, A. V., Skriabine, S., Lu, P., Weizman, O. E., Liu, F., Dai, Y., et al. (2020). Neuroinvasion of SARS-CoV-2 in human and mouse brain. bioRxiv.
Sterlin, D., Mathian, A., Miyara, M., Mohr, A., Anna, F., Claer, L., Quentric, P., Fadlallah, J., Devilliers, H., Ghillani, P., et al. (2020). IgA dominates the early neutralizing antibody response to SARS-CoV-2. Sci Transl Med.
Sternberg, A., and Naujokat, C. (2020). Structural features of coronavirus SARS-CoV-2 spike protein: Targets for vaccination. Life Sci 257, 118056.
Tostanoski, L. H., Wegmann, F., Martinot, A. J., Loos, C., McMahan, K., Mercado, N. B., Yu, J., Chan, C. N., Bondoc, S., Starke, C. E., et al. (2020). Ad26 vaccine protects against SARS-CoV-2 severe clinical disease in hamsters. Nat Med 26, 1694-1700.
Tseng, C. T., Huang, C., Newman, P., Wang, N., Narayanan, K., Watts, D. M., Makino, S., Packard, M. M., Zaki, S. R., Chan, T. S., et al. (2007). Severe acute respiratory syndrome coronavirus infection of mice transgenic for the human Angiotensin-converting enzyme 2 virus receptor. J Virol 81, 1162-1173. VandenDriessche T. et al. Blood, 1 Aug. 2002—vol. 100, no 3, p. 813-822
von Weyhern, C. H., Kaufmann, I., Neff, F., and Kremer, M. (2020). Early evidence of pronounced brain involvement in fatal COVID-19 outcomes. Lancet 395, e109.
Walls A. C., Park, Y. J., Tortorici, M. A., Wall, A., McGuire, A. T., and Veesler, D. (2020). Structure, Function, and Antigenicity of the SARS-CoV-2 Spike Glycoprotein. Cell 181, 281-292 e286.
Whittaker, A., Anson, M., and Harky, A. (2020). Neurological Manifestations of COVID-19: A systematic review and current update. Acta Neurol Scand 142, 14-22.
Wolfel, R., Corman, V. M., Guggemos, W., Seilmaier, M., Zange, S., Muller, M. A., Niemeyer, D., Jones, T. C., Vollmar, P., Rothe, C., et al. (2020). Virological assessment of hospitalized patients with COVID-2019. Nature 581, 465-469.
Xu, J., and Lazartigues, E. (2020). Expression of ACE2 in Human Neurons Supports the Neuro-Invasive Potential of COVID-19 Virus. Cell Mol Neurobiol.
Yang, X. H., Deng, W., Tong, Z., Liu, Y. X., Zhang, L. F., Zhu, H., Gao, H., Huang, L., Liu, Y. L., Ma, C. M., et al. (2007). Mice transgenic for human angiotensin-converting enzyme 2 provide a model for SARS coronavirus infection. Comp Med 57, 450-459.
Zennou, V., Petit, C., Guetard, D., Nerhbass, U., Montagnier, L., and Charneau, P. (2000). HIV-1 genome nuclear import is mediated by a central DNA flap. Cell 101, 173-185.

### REFERENCES CITED FOR EXAMPLES 4 AND 5

MBuss, L. F., Prete, C. A., Jr., Abrahim, C. M. M., Mendrone, A., Jr., Salomon, T., de Almeida-Neto, C., Franca, R. F. O., Belotti, M. C., Carvalho, M., Costa, A. G., et al. (2021). Three-quarters attack rate of SARS-CoV-2 in the Brazilian Amazon during a largely unmitigated epidemic. Science 371, 288-292.
Hoffmann, M., Arora, P., Gross, R., Seidel, A., Hornich, B. F., Hahn, A. S., Kruger, N., Graichen, L., Hofmann-Winkler, H., Kempf, A., et al. (2021). SARS-CoV-2 variants B.1.351 and P.1 escape from neutralizing antibodies. Cell.
Kitamura, D., Roes, J., Kuhn, R., and Rajewsky, K. (1991). A B cell-deficient mouse by targeted disruption of the membrane exon of the immunoglobulin mu chain gene. Nature 350, 423-426.
Ku, M. W., Bourgine, M., Authie, P., Lopez, J., Nemirov, K., Moncoq, F., Noirat, A., Vesin, B., Nevo, F., Blanc, C., et al. (2021). Intranasal vaccination with a lentiviral vector protects against SARS-CoV-2 in preclinical animal models. Cell Host Microbe 29, 236-249 e236.

1. A method of inducing and/or activating a protective immune response against Severe Acute Respiratory Syndrome beta-coronavirus 2 (SARS-CoV-2) in a subject, comprising administering to the upper respiratory tract of the subject an effective amount of an agent that induces a protective immune response against SARS-CoV-2.

2. The method of claim 1, wherein the agent that induces a protective immune response against SARS-CoV-2 is a pseudotyped lentiviral vector particle encoding a Severe Acute Respiratory Syndrome beta-coronavirus 2 (SARS-CoV-2) spike (S) protein or a derivative or fragment thereof.

3. The method of claim 1 or 2, wherein the agent is administered by aerosol inhalation.

4. The method of claim 2, wherein the agent is administered by nasal instillation.

5. The method of claim 2, wherein the agent is administered by nasal insufflation.

6. The method of any one of claims 1 to 5, wherein the treatment course consists of a single administration to the upper respiratory tract or wherein the treatment course comprises more than one administration, in particular two administrations, to the upper respiratory tract.

7. The method of any one of claims 1 to 5, wherein the treatment course comprises at least one priming administration outside of the respiratory tract, such as intramuscular, intradermal or subcutaneous routes, followed by at least one boosting administration to the upper respiratory tract.

8. The method of any one of claims 1 to 7, wherein the protective immune response comprises production of SARS-CoV-2 neutralizing antibodies in the subject.

9. The method of claim 8, wherein the neutralizing antibodies comprise IgG antibodies.

10. The method of any one of claims 1 to 9, wherein the protective immune response comprises production of SARS-CoV-2 S-specific T cells in the subject.

11. The method of claim 10, wherein the SARS-CoV-2 S-specific T cells comprise CD4⁺ T cells, CD8⁺ T cells, or both CD4⁺ and CD8⁺ T cells.

12. The method of claim 10 or 11, wherein the SARS-CoV-2 S-specific T cells comprise lung CD8⁺ T cells.

13. The method of any one of claims 10 to 12, wherein the SARS-CoV-2 S-specific T cells comprise IFN-γ-producing T-cells.

14. The method of any one of claims 10 to 13, wherein the CD8⁺ T cells comprise T cells with an effector memory (Tₑₘ) and/or resident memory (Tᵣₘ) phenotype.

15. The method of any one of claims 10 to 14, wherein the SARS-CoV-2 S-specific T cells are recruited to the olfactory bulb.

16. The method of any one of claims 1 to 15 wherein the protective immune response provides a reduced likelihood of developing SARS-CoV-2 infection-related inflammation in the subject.

17. The method of any one of claims 2 to 16, wherein the SARS-CoV-2 S protein has an amino acid sequence identical to SEQ ID NO: 1 and the SARS-CoV-2 S protein derivative has an amino acid sequence at least 95% identical to SEQ ID NO: 1.

18. The method of any one of claims 2 to 17, wherein the SARS-CoV-2 S protein is expressed from a coding sequence having a nucleotide sequence identical to SEQ ID NO: 2 and the SARS-CoV-2 S protein derivative is expressed from a coding sequence having a nucleotide sequence at least 80% identical to SEQ ID NO: 2.

19. The method of any one of claims 2 to 18, wherein the SARS-CoV-2 S protein derivative or fragment thereof comprises a peptide selected from peptide 61-75 (NVTWFHAIHVSGTNG (SEQ ID NO: 15)), peptide 536-550 (NKCVNFNFNGLTGTG (SEQ ID NO: 16)) and peptide 576-590 (VRDPQTLEILDITPC (SEQ ID NO: 17)).

20. The method of any one of claims 2 to 18, wherein the SARS-CoV-2 S derivative or fragment thereof comprises an amino acid modification relative to SEQ ID NO: 1, the modification selected from:
(i) 986^{K→P} and 987^{V→P},
(ii) 681^{PRRARS}686 (SEQ ID NO: 22)→681^{PGSAGS}686 (SEQ ID NO: 23), and
(iii) 986^{K→}P, 987V^{→P}, and 675^{QTQTNSPRRAR}685 (SEQ ID NO: 24) deletion.

21. The method of any one of claims 2 to 20, wherein the Severe Acute Respiratory Syndrome beta-coronavirus 2 (SARS-CoV-2) spike (S) protein or a derivative or fragment thereof comprises or consists of an amino acid sequence selected from SEQ ID NOS: 1, 5, 8, 11, 14, 108, 111, 114, 117, and 120.

22. The method of any one of claims 2 to 21, wherein the administered lentiviral vector particle is integrative.

23. The method of any one of claims 2 to 21, wherein the administered lentiviral vector particle is nonintegrative.

24. The method of claim 23, wherein the administered nonintegrative lentiviral particle comprises a D64V mutation in an integrase coding sequence.

25. The method of any one of claims 2 to 24, wherein the administered lentiviral vector particle is pseudotyped with Vesicular Stomatitis Virus envelop Glycoprotein (VSV-G).

26. The method of any one of claims 2 to 25, wherein lentiviral vector particle is administered as a vaccine formulation comprising the lentiviral vector particle and a pharmaceutically acceptable carrier.

27. A dosage form for administration to the upper respiratory tract of a subject of a pseudotyped lentiviral vector particle encoding a Severe Acute Respiratory Syndrome beta-coronavirus 2 (SARS-CoV-2) spike (S) protein or a derivative or fragment thereof.

28. The dosage form of claim 27, wherein the dosage form is for administration by aerosol inhalation.

29. The dosage form of claim 27, wherein the dosage form is for administration by nasal instillation.

30. The dosage form of claim 27, wherein the dosage form is for administration by nasal insufflation.

31. The dosage form of any one of claims 27 to 30, wherein the SARS-CoV-2 S protein has an amino acid sequence identical to SEQ ID NO: 1 and the SARS-CoV-2 S protein derivative has an amino acid sequence at least 95% identical to SEQ ID NO: 1.

32. The dosage form of any one of claims 27 to 30, wherein the SARS-CoV-2 S protein is expressed from a coding sequence having a nucleotide sequence identical to SEQ ID NO: 2 and the SARS-CoV-2 S protein derivative is expressed from a coding sequence having a nucleotide sequence at least 80% identical to SEQ ID NO: 2.

33. The dosage form of any one of claims 27 to 32, wherein the SARS-CoV-2 S protein derivative or fragment thereof comprises a peptide selected from peptide 61-75 (NVTWFHAIHVSGTNG (SEQ ID NO: 15)), peptide 536-550 (NKCVNFNFNGLTGTG (SEQ ID NO: 16)) and peptide 576-590 (VRDPQTLEILDITPC (SEQ ID NO: 17)).

34. The dosage form of any one of claims 27 to 33, wherein the SARS-CoV-2 S derivative or fragment thereof comprises an amino acid modification relative to SEQ ID NO: 1, the modification selected from:
(i) 986^{K→P} and 987^{V→P},
(ii) 681^{PRRARS}686 (SEQ ID NO: 22)→681^{PGSAGS}686 (SEQ ID NO: 23), and
(iii) 986^{K→P}, 987^{V→P}, and 675^{QTQTNSPRRAR}685 (SEQ ID NO: 24) deletion.

35. The dosage form of any one of claims 27 to 34, wherein the Severe Acute Respiratory Syndrome beta-coronavirus 2 (SARS-CoV-2) spike (S) protein or a derivative or fragment thereof comprises or consists of an amino acid sequence selected from SEQ ID NOS: 1, 5, 8, 11, 14, 108, 111, 114, 117, and 120

36. The dosage form of any one of claims 27 to 35, wherein the administered lentiviral vector particle is integrative.

37. The dosage form of any one of claims 27 to 35, wherein the administered lentiviral vector particle is nonintegrative.

38. The dosage form of claim 37, wherein the nonintegrative lentiviral particle comprises a D64V mutation in an integrase coding sequence.

39. The dosage form of any one of claims 27 to 38, wherein the lentiviral vector particle is pseudotyped with Vesicular Stomatitis Virus envelop Glycoprotein (VSV-G).

40. A kit comprising the dosage form of the pseudotyped lentiviral vector particle encoding a SARS-CoV-2 S protein or a derivative or fragment thereof according to any one of claims 27 to 39 and an applicator for administration to the upper respiratory tract.

41. The kit of claim 40, wherein the applicator for administration to the upper respiratory tract is an applicator for aerosol inhalation.

42. The kit of claim 40, wherein the applicator for administration to the upper respiratory tract is an applicator for nasal instillation.

43. The kit of claim 470, wherein the applicator for administration to the upper respiratory tract is an applicator for nasal insufflation.

44. A vector selected from:
pFlap-ieCMV-S2PdeltaF-WPREm (CNCM I-5537),
pFlap-ieCMV-S2P3F-WPREm (CNCM I-5538),
pFlap-ieCMV-S2P-WPREm (CNCM I-5539),
pFlap-ieCMV-SFL-WPREm (CNCM I-5540),
pFlap-ieCMV-S-B1.1.7-WPREm (CNCM I-5708),
pFlap-ieCMV-S-B351-WPREm (CNCM I-5709),
pFlap-ieCMV-S-B351-2P-WPREm (CNCM I-5710),
pFlap-ieCMV-SFL-D614G-WPREm (CNCM I-5711), and
pFlap-ieCMV-S-P1-WPREm (CNCM I-5712).

45. A host cell comprising a vector of claim 38.

46. A pseudotyped lentiviral vector particle encoding a Severe Acute Respiratory Syndrome beta-coronavirus 2 (SARS-CoV-2) spike (S) protein or a derivative or fragment thereof.

47. A pseudotyped lentiviral vector particle according to claim 46 wherein the encoded SARS-CoV-2 spike protein derivative or fragment thereof is as defined in any one of claim 31, 32, 33, 34 or 35.

48. A pseudotyped lentiviral vector particle according to claim 46 or 47 wherein the SARS-CoV-2 spike protein is selected from the SARS-CoV-2 spike protein that has the amino acid sequence of SEQ ID No. 1; the SARS-CoV-2 S protein derivative that has an amino acid sequence at least 95% identical or at least 99% identical to SEQ ID NO:1; the SARS-CoV-2 spike protein derivative that has the amino acid sequence of SEQ ID NO: 8, SEQ ID No. 11, SEQ ID No. 108, SEQ ID No. 111, SEQ ID No. 114, SEQ ID No. 117, or SEQ ID No. 120; the SARS-CoV-2 S protein derivative that has an amino acid sequence at least 95% identical or at least 99% identical to SEQ ID NO: 8, SEQ ID No. 11, SEQ ID No. 108, SEQ ID No. 111, SEQ ID No. 114, SEQ ID No. 117, or SEQ ID No. 120; and the SARS-CoV-2 spike protein fragment that has the amino acid sequence of SEQ ID No. 14 or the SARS-CoV-2 S protein derivative that has an amino acid sequence at least 95% identical or at least 99% identical to SEQ ID NO: 14.

49. A pseudotyped lentiviral vector particle according to any one of claims 46 to 48 wherein the pseudotyped lentiviral vector particle is as defined in any one of claim 36, 37, 38, or 39.

50. A pseudotyped lentiviral vector particle encoding a Severe Acute Respiratory Syndrome beta-coronavirus 2 (SARS-CoV-2) spike (S) protein or a derivative or fragment thereof, wherein the pseudotyped lentiviral vector particle is made by a method comprising co-transfection of a host cell with a vector selected from:
pFlap-ieCMV-S2PdeltaF-WPREm (CNCM I-5537),
pFlap-ieCMV-S2P3F-WPREm (CNCM I-5538),
pFlap-ieCMV-S2P-WPREm (CNCM I-5539),
pFlap-ieCMV-SFL-WPREm (CNCM I-5540),
pFlap-ieCMV-S-B1.1.7-WPREm (CNCM I-5708),
pFlap-ieCMV-S-B351-WPREm (CNCM I-5709),
pFlap-ieCMV-S-B351-2P-WPREm (CNCM I-5710),
pFlap-ieCMV-SFL-D614G-WPREm (CNCM I-5711), and
pFlap-ieCMV-S-P1-WPREm (CNCM I-5712).

51. A pseudotyped lentiviral vector particle according to any one of claims 46 to 49, wherein the genome of the vector particle comprises a polynucleotide selected from:
a polynucleotide encoding S2PΔF (S2PdeltaF) of SEQ ID No. 13 or a coding sequence having a nucleotide sequence at least 80% identical to SEQ ID No.13, in particular a coding sequence having a mutation, in particular a deletion, in the RBD,
a polynucleotide encoding S2P3F of SEQ ID No. 10 or a coding sequence having a nucleotide sequence at least 80% identical to SEQ ID No.10 having a mutation in the RBD, in particular wherein the coding sequence having a nucleotide sequence at least 80% identical to SEQ ID No.10 comprises mutations 986^{K→P} and 987^{V→P}.
a polynucleotide encoding S2P of SEQ ID No. 7 or a coding sequence having a nucleotide sequence at least 80% identical to SEQ ID No.7 having a mutation in the RBD,
a polynucleotide encoding SFL of SEQ ID No. 2 or a coding sequence having a nucleotide sequence at least 80% identical to SEQ ID No. 2 having a mutation in the RBD,
a polynucleotide encoding S-B1.1.7 of SEQ ID No. 107 or a coding sequence having a nucleotide sequence at least 80% identical to SEQ ID No. 107 having a mutation in the RBD,
a polynucleotide encoding S-B351 of SEQ ID No. 110 or a coding sequence having a nucleotide sequence at least 80% identical to SEQ ID No. 110 having a mutation in the RBD,
a polynucleotide encoding S-B1.1.7 S-B351-2P of SEQ ID No. 113 or a coding sequence having a nucleotide sequence at least 80% identical to SEQ ID No. 113 having a mutation in the RBD,
a polynucleotide encoding SFL-D614G of SEQ ID No. 116 or a coding sequence having a nucleotide sequence at least 80% identical to SEQ ID No. 116 having a mutation in the RBD, and
a polynucleotide encoding S-P1 of SEQ ID No. 119 or a coding sequence having a nucleotide sequence at least 80% identical to SEQ ID No. 119 having a mutation in the RBD.

52. An immunogenic composition that comprises a dosage form according to any one of claims 27 to 39 or a pseudotyped lentiviral particle according to any one of claims 46 to 51.

53. An immunogenic composition according to claim 52 for use in inducing and/or activating a protective immune response against Severe Acute Respiratory Syndrome beta-coronavirus 2 (SARS-CoV-2) in a subject, wherein said use comprises a prime administration outside of the upper respiratory tract, in particular systemic, especially intramuscular administration and a boost or target administration to the upper respiratory tract.

54. The immunogenic composition according to claim 52 for use according to claim 53 wherein the administered doses or LV particles are identical in the prime and boost/target administration steps, or wherein the administered doses or LV particles are different in the prime and boost/target administration steps, in particular may be higher for the administration to the upper respiratory tract.

55. The immunogenic composition according to claim 52 for use according to claim 53 or 54 wherein the lentiviral vector particles are LV::SFL, in particular NILV::SFL and the administration regimen consists in a systemic, especially i.m. prime and a boost to the upper respiratory tract, in particular by i.n. boost.

56. The immunogenic composition according to claim 52 for use according to claim 53 or 54 wherein the lentiviral vector particles are LV::S_{prefusion}, in particular NILV::S_{prefusion}, such as LV::S2PΔF (LV::S2deltaF) or NILV::S2PΔF (NILV::S2deltaF), or LV::S2P3F or NILV::S2P3F and the administration regimen consists in a systemic, especially i.m. prime and a boost to the upper respiratory tract, in particular by i.n. boost.

57. The immunogenic composition according to claim 52 for use to induce a protective immune response against SARS-CoV-2 in the upper respiratory tract of a subject and/or in the brain against SARS-CoV-2.

58. The immunogenic composition according to claim 52 for use to induce a cross protective immune response of lungs and brain against ancestral including SARS-CoV-2 selected from the group of SARS-CoV-2 Wuhan strain, SARS-CoV-2 D614G strain and SARS-CoV-2 B1.117 strain and against emerging SARS-CoV-2 variants such as SARS-CoV-2 P.1 variant, by eliciting B and T cell-responses.

59. The immunogenic composition according to claim 52 for use according to claims 53 to 58 wherein the dosage form or the pseudotyped lentiviral particle comprises pseudotyped lentiviral particles according to any one of claims 46 to 51 wherein the pseudotyped lentiviral particles are non-integrative.

60. The immunogenic composition according to claim 52 for use according to claim 53 or 58 to elicit a protective immune response against SARS-CoV-2 wherein the response elicits SARS-CoV-2 S-specific T cells, in particular SARS-CoV-2 S-specific T cells that comprise lung CD8+ T cells and/or IFN-γ-producing T-cells.

61. The immunogenic composition according to claim 52 for use according to any one of claims 53 to 60 to elicit a protective immune response against SARS-CoV-2 wherein the response elicits CD8+ T cells that comprise T cells with an effector memory (Tₑₘ) and/or resident memory (Tᵣₘ) phenotype.

62. The immunogenic composition according to claim 52 for use according to any one of claims 53 to 61, the SARS-CoV-2 S-specific T cells are recruited to the olfactory bulb.

63. The immunogenic composition according to claim 52 for use according to claims 53 to 62 wherein the Severe Acute Respiratory Syndrome beta-coronavirus 2 (SARS-CoV-2) spike (S) protein or a derivative or fragment thereof comprises or consists of an amino acid sequence selected from SEQ ID NOS: 1, 5, 8, 11, 14, 108, 111, 114, 117, and 120.

64. The immunogenic composition according to claim 52 for use according to any one of claims 53 to 63 to prevent or to alleviate SARS-CoV-2 infection-related inflammation in the subject.


SEQUENCE LISTING (152 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 10079 bp, Artificial Sequence
  source: /note=“Description of Artificial Sequence: Synthetic polynucleotide”
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatcccgtacggccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataacgagcgcgcctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 4 - DNA, 3825 bp, Severe acute respiratory syndrome coronavirus 2
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 5 - protein, 1273 aa, Severe acute respiratory syndrome coronavirus 2; identical to SEQ ID NO: 1

SEQ ID NO: 6 - DNA, 10079 bp, Artificial Sequence
  source: /note=“Description of Artificial Sequence: Synthetic polynucleotide”
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatctcgtacggccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataacgagcgcgcctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 7 - DNA, 3825 bp, Artificial Sequence
  source: /note=“Description of Artificial Sequence: Synthetic polynucleotide”
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 8 - protein, 1273 aa, Artificial Sequence
  source: /note=“Description of Artificial Sequence: Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - DNA, 10079 bp, Artificial Sequence
  source: /note=“Description of Artificial Sequence: Synthetic polynucleotide”
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatctcgtacggccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccaggatccgccggaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataacgagcgcgcctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 10 - DNA, 3825 bp, Artificial Sequence
  source: /note=“Description of Artificial Sequence: Synthetic polynucleotide”
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccaggatccgccggaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 11 - protein, 1273 aa, Artificial Sequence
  source: /note=“Description of Artificial Sequence: Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - DNA, 10046 bp, Artificial Sequence
  source: /note=“Description of Artificial Sequence: Synthetic polynucleotide”
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatctcgtacggccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcatcctatagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataacgagcgcgcctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 13 - DNA, 3792 bp, Artificial Sequence
  source: /note=“Description of Artificial Sequence: Synthetic polynucleotide”
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcatcctatagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 14 - protein, 1262 aa, Artificial Sequence
  source: /note=“Description of Artificial Sequence: Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 15 aa, Severe acute respiratory syndrome coronavirus 2
NVTWFHAIHVSGTNG

SEQ ID NO: 16 - protein, 15 aa, Severe acute respiratory syndrome coronavirus 2
NKCVNFNFNGLTGTG

SEQ ID NO: 17 - protein, 15 aa, Severe acute respiratory syndrome coronavirus 2
VRDPQTLEILDITPC

SEQ ID NO: 18 - protein, 15 aa, Severe acute respiratory syndrome coronavirus 2
LDSKVGGNYNYLYRL

SEQ ID NO: 19 - protein, 15 aa, Severe acute respiratory syndrome coronavirus 2
CASYQTQTNSPRRAR

SEQ ID NO: 20 - protein, 15 aa, Severe acute respiratory syndrome coronavirus 2
VQIDRLITGRLQSLQ

SEQ ID NO: 21 - protein, 20 aa, Severe acute respiratory syndrome coronavirus 2
SGWTAGAAAYYVGYLQPRTF

SEQ ID NO: 22 - protein, 6 aa, Severe acute respiratory syndrome coronavirus 2
PRRARS

SEQ ID NO: 23 - protein, 6 aa, Artificial Sequence
  source: /note=“Description of Artificial Sequence: Synthetic peptide”
PGSAGS

SEQ ID NO: 24 - protein, 11 aa, Severe acute respiratory syndrome coronavirus 2
QTQTNSPRRAR

SEQ ID NO: 25 - DNA, 11342 bp, Artificial Sequence
  source: /note=“Description of Artificial Sequence: Synthetic polynucleotide”
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtatccagtgggggaatataaaggtgaaagcaggagagacccctctgactggaacctcttacctcccagaagccttgtatgcaaaaccagtgggcattcatttgtatgttattttgcatcccgtttgcctcccagccttcagcaggccccgaccctcccctggccagcttccaccctgactgccccctggctggctcccattgagcactgtgggctctccccaccattaggtgacagatcaggaacaatccaggctcaggctctttatctgtgctctgcctcccacctggcaggtccactggccaggcttttccagggtcccttctctcccaggtctgccctactatttgtcctccccttccccctcagctggtagctcgataagaatcaataggtccactccagagcaaagaacacagccaaatgtgtcataccaggccctgccagaaaaacgagctgctggagctgacaaacttgaaggccaaacacctaaggttccccccaacacttcattcagcagggatggtcattcagcttcagggggcaggcagcatgaaagcctccctacctccatccttctcacacagaggctggggagagcatcttggaggatgcagtcccctggggccaggcttctaatccagacagcccttacaaggggggacaggggaaggactggcttggagaaaagtcctagaaaagaggggaggggcactggccaccagggctgggtcgctgctatgatggtcctaggagtgcctgcctgtcctctcaggccccatgcgatgtaggacacattacttttatttatttatttatttattttgagtcagagtttcgctctggttgcccaggctggagcgcgacggcacgatcttggctcactgcaacctctgcctcctgggttcaagcgattctcctgcctcagcctcctgagtagctgggattacaggcacacactgtgctggttaatttttgtatttttagtagagaaggggtgtcaccatgttggtcaggctggtctcaaatttttttttttttttttttttttttttgagacagagtcttgctctgttgtctaggctggagtgcagtggcatcgaactcttgacctcaagtgatccacccgcctcggcctcccaaagtgcttggattacaggcatgagccactgtgcccggcgatgtgggacacattatcatctctgtgagagatttttggtctcttttgtcaccgcccttctctcccagctcctagaactgggcctggctcacagtaggtgctgaatgcatactggttgaattgtaaatgctcaggatttgtttaattaaggatgcaggaaaggtgatataccggtgtgcagaagtcaggatgcattccctgtccaaatcacagtgttccactgaggcaaggcccttgggagtgaggtcgggagaggggagggtggtggagggggctcagagactgggtttgttttggggagtctgcacctatttgctgagtgaatgtatgtgtgtgtgcatttgagagcacacctctgtatgattcgggtgtgagtgtgtgtgaggaaacgtgggcaggcgaggagtgtttgggagccaggtgcagctggggtgtgagtgtgtaagcaagcagctatgaggctgggcattgcttctcctcctcttctccagctcccagcctttcttccccgggactcctggggctccaggatgcccccaagatcccctccacaagtggataatttgggctgcaggttaaggacagctagagggactcacaggccattccacccgcacaccaccagacccccaaatttcttttttcttttttttttgagacagagtctcactctgtcgccaggctgcagtggcgcgatctcggctcactgcaacctccgcctcccaggttcaagcgattccccttcctcagcctcccaagtagctgagactacaggcgtgcaccatcacgtccggctaattttttgtattttagtagagaggggtttcaccatgttggctaggatggtctcgatctcctgacctcgtgatccgcccacctaggcctcccaaagtgctgagattacaggcgtgagccactgcgcccggtcaagactcccaaatttcaaactcgccagcacctcctccacctgggggagaagagcataataacgtcatttcctgccctgaaagcagcctcgagggccaacaacacctgctgtccgtgtccatgcccggttggccaccccgtttctggggggtgagcggggcttggcagggctgcgcggagggcgcgggggtggggcccggggcggagcggcccggggcggagggcgcgggctccgagccgtccacctgtggctccggcttccgaagcggctccggggcgggggcggggcctcactctgcgatataactcgggtcgcgcggctcgcgcaggccgccaccgtcgtccgcaaagcctgagtcctgtcctttctctctccccggacagcaatcgatagggctcaggtaagtggtaggagggacctcaactcccagccttgtctgaccctccaattatacactcctttgcctctttccgtcattccataaccaccccaacccctactccaccgggagggggttgggcatacctggatttccatccgcgcacctagccacagggtccctaagagcagcagcagctaggcatgggagggctctttcccaggagagagggggaaggggacagggttgagagctttacagaggaagtggacagcatggagggaggtaaggaaaggcctgtaaagaggaggagacactggctctggcggaatggggactattggagggttaagcggatgtggctaaggctgagtcatctaggagtaaacaagaggccttcctttgggaggagccaatccagggtgtagggggcccagagtgaccaggtgcactagggaaaaaatgccaggagagggccaggaagaggacttgttagtagcgactcacttctgggcaggcaggccagccagctagccagcctgctgaggcttcccaagaggggcagagtgctgggatctgggaatccaggaaaggagggaatggggtggggctagatgaaaagggataggtgtccagggagagcctctggctattcctgggaccaggaagttttcactaggatacataacactttttacacactcaccccacccatccctggctttctattcatggaacaacctctctcctttttccttccaggtggatccgccaccatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttaactcgagctcaagcttcgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattctgcagtcgacggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 26 - DNA, 3249 bp, Artificial Sequence
  source: /note=“Description of Artificial Sequence: Synthetic polynucleotide”
atccagtgggggaatataaaggtgaaagcaggagagacccctctgactggaacctcttacctcccagaagccttgtatgcaaaaccagtgggcattcatttgtatgttattttgcatcccgtttgcctcccagccttcagcaggccccgaccctcccctggccagcttccaccctgactgccccctggctggctcccattgagcactgtgggctctccccaccattaggtgacagatcaggaacaatccaggctcaggctctttatctgtgctctgcctcccacctggcaggtccactggccaggcttttccagggtcccttctctcccaggtctgccctactatttgtcctccccttccccctcagctggtagctcgataagaatcaataggtccactccagagcaaagaacacagccaaatgtgtcataccaggccctgccagaaaaacgagctgctggagctgacaaacttgaaggccaaacacctaaggttccccccaacacttcattcagcagggatggtcattcagcttcagggggcaggcagcatgaaagcctccctacctccatccttctcacacagaggctggggagagcatcttggaggatgcagtcccctggggccaggcttctaatccagacagcccttacaaggggggacaggggaaggactggcttggagaaaagtcctagaaaagaggggaggggcactggccaccagggctgggtcgctgctatgatggtcctaggagtgcctgcctgtcctctcaggccccatgcgatgtaggacacattacttttatttatttatttatttattttgagtcagagtttcgctctggttgcccaggctggagcgcgacggcacgatcttggctcactgcaacctctgcctcctgggttcaagcgattctcctgcctcagcctcctgagtagctgggattacaggcacacactgtgctggttaatttttgtatttttagtagagaaggggtgtcaccatgttggtcaggctggtctcaaatttttttttttttttttttttttttttgagacagagtcttgctctgttgtctaggctggagtgcagtggcatcgaactcttgacctcaagtgatccacccgcctcggcctcccaaagtgcttggattacaggcatgagccactgtgcccggcgatgtgggacacattatcatctctgtgagagatttttggtctcttttgtcaccgcccttctctcccagctcctagaactgggcctggctcacagtaggtgctgaatgcatactggttgaattgtaaatgctcaggatttgtttaattaaggatgcaggaaaggtgatataccggtgtgcagaagtcaggatgcattccctgtccaaatcacagtgttccactgaggcaaggcccttgggagtgaggtcgggagaggggagggtggtggagggggctcagagactgggtttgttttggggagtctgcacctatttgctgagtgaatgtatgtgtgtgtgcatttgagagcacacctctgtatgattcgggtgtgagtgtgtgtgaggaaacgtgggcaggcgaggagtgtttgggagccaggtgcagctggggtgtgagtgtgtaagcaagcagctatgaggctgggcattgcttctcctcctcttctccagctcccagcctttcttccccgggactcctggggctccaggatgcccccaagatcccctccacaagtggataatttgggctgcaggttaaggacagctagagggactcacaggccattccacccgcacaccaccagacccccaaatttcttttttcttttttttttgagacagagtctcactctgtcgccaggctgcagtggcgcgatctcggctcactgcaacctccgcctcccaggttcaagcgattccccttcctcagcctcccaagtagctgagactacaggcgtgcaccatcacgtccggctaattttttgtattttagtagagaggggtttcaccatgttggctaggatggtctcgatctcctgacctcgtgatccgcccacctaggcctcccaaagtgctgagattacaggcgtgagccactgcgcccggtcaagactcccaaatttcaaactcgccagcacctcctccacctgggggagaagagcataataacgtcatttcctgccctgaaagcagcctcgagggccaacaacacctgctgtccgtgtccatgcccggttggccaccccgtttctggggggtgagcggggcttggcagggctgcgcggagggcgcgggggtggggcccggggcggagcggcccggggcggagggcgcgggctccgagccgtccacctgtggctccggcttccgaagcggctccggggcgggggcggggcctcactctgcgatataactcgggtcgcgcggctcgcgcaggccgccaccgtcgtccgcaaagcctgagtcctgtcctttctctctccccggacagcaatcgatagggctcaggtaagtggtaggagggacctcaactcccagccttgtctgaccctccaattatacactcctttgcctctttccgtcattccataaccaccccaacccctactccaccgggagggggttgggcatacctggatttccatccgcgcacctagccacagggtccctaagagcagcagcagctaggcatgggagggctctttcccaggagagagggggaaggggacagggttgagagctttacagaggaagtggacagcatggagggaggtaaggaaaggcctgtaaagaggaggagacactggctctggcggaatggggactattggagggttaagcggatgtggctaaggctgagtcatctaggagtaaacaagaggccttcctttgggaggagccaatccagggtgtagggggcccagagtgaccaggtgcactagggaaaaaatgccaggagagggccaggaagaggacttgttagtagcgactcacttctgggcaggcaggccagccagctagccagcctgctgaggcttcccaagaggggcagagtgctgggatctgggaatccaggaaaggagggaatggggtggggctagatgaaaagggataggtgtccagggagagcctctggctattcctgggaccaggaagttttcactaggatacataacactttttacacactcaccccacccatccctggctttctattcatggaacaacctctctcctttttccttccaggt

SEQ ID NO: 27 - DNA, 9 bp, Artificial Sequence
  source: /note=“Description of Artificial Sequence: Synthetic oligonucleotide”
aagtggtag

SEQ ID NO: 28 - DNA, 16 bp, Artificial Sequence
  source: /note=“Description of Artificial Sequence: Synthetic oligonucleotide”
ctttttccttccaggt

SEQ ID NO: 29 - DNA, 2418 bp, Homo sapiens
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttaa

SEQ ID NO: 30 - protein, 859 aa, Homo sapiens
MKRDRCPGRASGYSWDQEVFTRIHNTFYTLTPPIPGFLFMEQPLSFFLPGGSATMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 31 - DNA, 605 bp, Artificial Sequence
  source: /note=“Description of Artificial Sequence: Synthetic polynucleotide”
aattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggg

SEQ ID NO: 32 - protein, 20 aa, Severe acute respiratory syndrome coronavirus 2
SGWTAGAAAYYVGYLQPRTF

SEQ ID NO: 33 - protein, 263 aa, Artificial Sequence
  source: /note=“Description of Artificial Sequence: Synthetic polypeptide”
IEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 34 - DNA, 26 bp, Artificial Sequence
  source: /note=“Description of Artificial Sequence: Synthetic primer”
acaggtacgttaatagttaatagcgt

SEQ ID NO: 35 - DNA, 22 bp, Artificial Sequence
  source: /note=“Description of Artificial Sequence: Synthetic primer”
atattgcagcagtacgcacaca

SEQ ID NO: 36 - DNA, 26 bp, Artificial Sequence
  source: /note=“Description of Artificial Sequence: Synthetic probe”
acactagccatccttactgcgcttcg

SEQ ID NO: 37 - DNA, 20 bp, Artificial Sequence
  source: /note=“Description of Artificial Sequence: Synthetic primer”
atgggaagccgaacatactg

SEQ ID NO: 38 - DNA, 20 bp, Artificial Sequence
  source: /note=“Description of Artificial Sequence: Synthetic primer”
cagtctcagtgggggtgaat

SEQ ID NO: 39 - DNA, 20 bp, Artificial Sequence
  source: /note=“Description of Artificial Sequence: Synthetic primer”
ttcaccaccatggagaaggc

SEQ ID NO: 40 - DNA, 20 bp, Artificial Sequence
  source: /note=“Description of Artificial Sequence: Synthetic primer”
ggcatggactgtggtcatga

SEQ ID NO: 41 - DNA, 22 bp, Artificial Sequence
  source: /note=“Description of Artificial Sequence: Synthetic primer”
ggatgtgaccttcctcagactc

SEQ ID NO: 42 - DNA, 22 bp, Artificial Sequence
  source: /note=“Description of Artificial Sequence: Synthetic primer”
accttctcctgcgggaatccaa

SEQ ID NO: 43 - DNA, 24 bp, Artificial Sequence
  source: /note=“Description of Artificial Sequence: Synthetic primer”
tcaagtggcatagatgtggaagaa

SEQ ID NO: 44 - DNA, 21 bp, Artificial Sequence
  source: /note=“Description of Artificial Sequence: Synthetic primer”
tggctctgcaggattttcatg

SEQ ID NO: 45 - DNA, 25 bp, Artificial Sequence
  source: /note=“Description of Artificial Sequence: Synthetic primer”
catcttctcaaaattcgagtgacaa

SEQ ID NO: 46 - DNA, 23 bp, Artificial Sequence
  source: /note=“Description of Artificial Sequence: Synthetic primer”
tgggagtagacaaggtacaaccc

SEQ ID NO: 47 - DNA, 22 bp, Artificial Sequence
  source: /note=“Description of Artificial Sequence: Synthetic primer”
tgacgtcactggagttgtacgg

SEQ ID NO: 48 - DNA, 21 bp, Artificial Sequence
  source: /note=“Description of Artificial Sequence: Synthetic primer”
ggttcatgtcatggatggtgc

SEQ ID NO: 49 - DNA, 22 bp, Artificial Sequence
  source: /note=“Description of Artificial Sequence: Synthetic primer”
tggaccttccaggatgaggaca

SEQ ID NO: 50 - DNA, 22 bp, Artificial Sequence
  source: /note=“Description of Artificial Sequence: Synthetic primer”
gttcatctcggagcctgtagtg

SEQ ID NO: 51 - DNA, 23 bp, Artificial Sequence
  source: /note=“Description of Artificial Sequence: Synthetic primer”
cctgagcaggatggagaattaca

SEQ ID NO: 52 - DNA, 19 bp, Artificial Sequence
  source: /note=“Description of Artificial Sequence: Synthetic primer”
tccagaacatgccgcagag

SEQ ID NO: 53 - DNA, 20 bp, Artificial Sequence
  source: /note=“Description of Artificial Sequence: Synthetic primer”
cgaggtcacaggagaaggga

SEQ ID NO: 54 - DNA, 22 bp, Artificial Sequence
  source: /note=“Description of Artificial Sequence: Synthetic primer”
aagccctacagacgagctcact

SEQ ID NO: 55 - DNA, 22 bp, Artificial Sequence
  source: /note=“Description of Artificial Sequence: Synthetic primer”
gatgaggcttcctgtccctact

SEQ ID NO: 56 - DNA, 25 bp, Artificial Sequence
  source: /note=“Description of Artificial Sequence: Synthetic primer”
tgacaggttttggaatagcatttcc

SEQ ID NO: 57 - DNA, 23 bp, Artificial Sequence
  source: /note=“Description of Artificial Sequence: Synthetic primer”
ctgcaagtgcatcatcgttgttc

SEQ ID NO: 58 - DNA, 22 bp, Artificial Sequence
  source: /note=“Description of Artificial Sequence: Synthetic primer”
taccacttcacaagtcggaggc

SEQ ID NO: 59 - DNA, 20 bp, Artificial Sequence
  source: /note=“Description of Artificial Sequence: Synthetic primer”
ggttgccaagccttatcgga

SEQ ID NO: 60 - DNA, 20 bp, Artificial Sequence
  source: /note=“Description of Artificial Sequence: Synthetic primer”
acctgctccactgccttgct

SEQ ID NO: 61 - DNA, 20 bp, Artificial Sequence
  source: /note=“Description of Artificial Sequence: Synthetic primer”
ggaagcacggcagcagaata

SEQ ID NO: 62 - DNA, 24 bp, Artificial Sequence
  source: /note=“Description of Artificial Sequence: Synthetic primer”
aacttgagggagaagtaggaatgg

SEQ ID NO: 63 - DNA, 17 bp, Artificial Sequence
  source: /note=“Description of Artificial Sequence: Synthetic primer”
gaagctcagtgccgcca

SEQ ID NO: 64 - DNA, 21 bp, Artificial Sequence
  source: /note=“Description of Artificial Sequence: Synthetic primer”
ttcatgtggtggtccagcttt

SEQ ID NO: 65 - DNA, 23 bp, Artificial Sequence
  source: /note=“Description of Artificial Sequence: Synthetic primer”
gacagcctgtgttcgaggatatg

SEQ ID NO: 66 - DNA, 23 bp, Artificial Sequence
  source: /note=“Description of Artificial Sequence: Synthetic primer”
tgttcttacaggagagggtagac

SEQ ID NO: 67 - DNA, 23 bp, Artificial Sequence
  source: /note=“Description of Artificial Sequence: Synthetic primer”
ctactgcatgagactccgttctg

SEQ ID NO: 68 - DNA, 22 bp, Artificial Sequence
  source: /note=“Description of Artificial Sequence: Synthetic primer”
agaatcccgtggataggcagag

SEQ ID NO: 69 - DNA, 20 bp, Artificial Sequence
  source: /note=“Description of Artificial Sequence: Synthetic primer”
aggtccctgtcatgcttctg

SEQ ID NO: 70 - DNA, 20 bp, Artificial Sequence
  source: /note=“Description of Artificial Sequence: Synthetic primer”
tctggacccattccttcttg

SEQ ID NO: 71 - DNA, 20 bp, Artificial Sequence
  source: /note=“Description of Artificial Sequence: Synthetic primer”
cctctgtcacctgctcaaca

SEQ ID NO: 72 - DNA, 20 bp, Artificial Sequence
  source: /note=“Description of Artificial Sequence: Synthetic primer”
gatgaattggcgtggaatct

SEQ ID NO: 73 - DNA, 20 bp, Artificial Sequence
  source: /note=“Description of Artificial Sequence: Synthetic primer”
gtgcccacgtcaaggagtat

SEQ ID NO: 74 - DNA, 20 bp, Artificial Sequence
  source: /note=“Description of Artificial Sequence: Synthetic primer”
gggaagcgtatacagggtca

SEQ ID NO: 75 - DNA, 24 bp, Artificial Sequence
  source: /note=“Description of Artificial Sequence: Synthetic primer”
gcatttctgttgctgttcacgctg

SEQ ID NO: 76 - DNA, 25 bp, Artificial Sequence
  source: /note=“Description of Artificial Sequence: Synthetic primer”
cctccttctggtttttcagtttagc

SEQ ID NO: 77 - DNA, 20 bp, Artificial Sequence
  source: /note=“Description of Artificial Sequence: Synthetic primer”
aaaatttcatcacgcccttg

SEQ ID NO: 78 - DNA, 20 bp, Artificial Sequence
  source: /note=“Description of Artificial Sequence: Synthetic primer”
tctccagcttggtgaggtct

SEQ ID NO: 79 - DNA, 20 bp, Artificial Sequence
  source: /note=“Description of Artificial Sequence: Synthetic primer”
ggatggctgtcctagctctg

SEQ ID NO: 80 - DNA, 20 bp, Artificial Sequence
  source: /note=“Description of Artificial Sequence: Synthetic primer”
ataaccccttgggaagatgg

SEQ ID NO: 81 - skipped

SEQ ID NO: 82 - DNA, 11 bp, Homo sapiens
tacaatccctc

SEQ ID NO: 83 - DNA, 11 bp, Artificial Sequence
  source: /note=“Description of Artificial Sequence: Synthetic oligonucleotide”
ttttttttttt

SEQ ID NO: 84 - DNA, 5 bp, Artificial Sequence
  source: /note=“Description of Artificial Sequence: Synthetic oligonucleotide”
caggt

SEQ ID NO: 85 - DNA, 19 bp, Artificial Sequence
  source: /note=“Description of Artificial Sequence: Synthetic primer”
tcctaaccagccccctgtt

SEQ ID NO: 86 - DNA, 23 bp, Artificial Sequence
  source: /note=“Description of Artificial Sequence: Synthetic primer”
tgacaatgccaaccactatcact

SEQ ID NO: 87 - DNA, 19 bp, Artificial Sequence
  source: /note=“Description of Artificial Sequence: Synthetic primer”
ggctgctgagcgtctggta

SEQ ID NO: 88 - DNA, 19 bp, Artificial Sequence
  source: /note=“Description of Artificial Sequence: Synthetic primer”
ccaggtcctgcgtgtctga

SEQ ID NO: 89 - DNA, 20 bp, Artificial Sequence
  source: /note=“Description of Artificial Sequence: Synthetic primer”
gcccagaacatcatccctgc

SEQ ID NO: 90 - DNA, 21 bp, Artificial Sequence
  source: /note=“Description of Artificial Sequence: Synthetic primer”
ccgttcagctctgggatgacc

SEQ ID NO: 91 - DNA, 26 bp, Artificial Sequence
  source: /note=“Description of Artificial Sequence: Synthetic primer”
acaggtacgttaatagttaatagcgt

SEQ ID NO: 92 - DNA, 22 bp, Artificial Sequence
  source: /note=“Description of Artificial Sequence: Synthetic primer”
atattgcagcagtacgcacaca

SEQ ID NO: 93 - DNA, 26 bp, Artificial Sequence
  source: /note=“Description of Artificial Sequence: Synthetic probe”
acactagccatccttactgcgcttcg

SEQ ID NO: 94 - DNA, 23 bp, Artificial Sequence
  source: /note=“Description of Artificial Sequence: Synthetic primer”
cgatctcttgtagatctgttctc

SEQ ID NO: 95 - DNA, 6 bp, Artificial Sequence
  source: /note=“Description of Artificial Sequence: Synthetic oligonucleotide”
aagtgg

SEQ ID NO: 96 - DNA, 12 bp, Artificial Sequence
  source: /note=“Description of Artificial Sequence: Synthetic oligonucleotide”
ctttttccttcc

SEQ ID NO: 97 - DNA, 6 bp, Artificial Sequence
  source: /note=“Description of Artificial Sequence: Synthetic oligonucleotide”
aagggg

SEQ ID NO: 98 - DNA, 605 bp, Artificial Sequence
  source: /note=“Description of Artificial Sequence: Synthetic polynucleotide”
aattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggg

SEQ ID NO: 99 - protein, 4 aa, Severe acute respiratory syndrome coronavirus 2
RRAR

SEQ ID NO: 100 - DNA, 20 bp, Artificial Sequence
  source: /note=“Description of Artificial Sequence: Synthetic primer”
tggaggaggagatatgaggg

SEQ ID NO: 101 - DNA, 20 bp, Artificial Sequence
  source: /note=“Description of Artificial Sequence: Synthetic primer”
ctgctgcactataccagaca

SEQ ID NO: 102 - DNA, 20 bp, Artificial Sequence
  source: /note=“Description of Artificial Sequence: Synthetic primer”
tctcctctgacttcaacagc

SEQ ID NO: 103 - DNA, 20 bp, Artificial Sequence
  source: /note=“Description of Artificial Sequence: Synthetic primer”
ccctgcactttttaagagcc

SEQ ID NO: 104 - protein, 8 aa, Artificial Sequence
  source: /note=“Description of Artificial Sequence: Synthetic 8xHis tag”
HHHHHHHH

SEQ ID NO: 105 - DNA, 18 bp, Artificial Sequence
  source: /note=“Description of Artificial Sequence: Synthetic primer”
tttttttttttttttttt

SEQ ID NO: 106 - DNA, 10077 bp, Artificial Sequence
  source: /note=“Description of Artificial Sequence: Synthetic polynucleotide”
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttcgcctggagacgccatccacgctgttttgacctccatagaagacaccgcgatcggatccacaagtttgtacaaaaaagcaggctgccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatctccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgacgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtcaccgacgagcccgaagtgtggccagccagagcatcattgcctataccatgagcctgggcgccgagaatagcgtggcctactccaacaacagcattgctatccccatcaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctggcccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccacccacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgatagtaaacccagctttcttgtacaaagtggtctcgagttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 107 - DNA, 3813 bp, Artificial Sequence
  source: /note=“Description of Artificial Sequence: Synthetic polynucleotide”
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatctccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgacgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtcaccgacgagcccgaagtgtggccagccagagcatcattgcctataccatgagcctgggcgccgagaatagcgtggcctactccaacaacagcattgctatccccatcaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctggcccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccacccacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 108 - protein, 1270 aa, Artificial Sequence
  source: /note=“Description of Artificial Sequence: Synthetic polypeptide”
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 109 - DNA, 10077 bp, Artificial Sequence
  source: /note=“Description of Artificial Sequence: Synthetic polynucleotide”
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttcgcctggagacgccatccacgctgttttgacctccatagaagacaccgcgatcggatccacaagtttgtacaaaaaagcaggctgccaccatgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgatagtaaacccagctttcttgtacaaagtggtctcgagttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 110 - DNA, 3813 bp, Artificial Sequence
  source: /note=“Description of Artificial Sequence: Synthetic polynucleotide”
atgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 111 - protein, 1270 aa, Artificial Sequence
  source: /note=“Description of Artificial Sequence: Synthetic polypeptide”
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 112 - DNA, 10096 bp, Artificial Sequence
  source: /note=“Description of Artificial Sequence: Synthetic polynucleotide”
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatccacaagtttgtacaaaaaagcaggctgccaccatgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgatagtaaacccagctttcttgtacaaagtggtctcgagttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 113 - DNA, 3813 bp, Artificial Sequence
  source: /note=“Description of Artificial Sequence: Synthetic polynucleotide”
atgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 114 - protein, 1270 aa, Artificial Sequence
  source: /note=“Description of Artificial Sequence: Synthetic polypeptide”
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 115 - DNA, 10064 bp, Artificial Sequence
  source: /note=“Description of Artificial Sequence: Synthetic polynucleotide”
cgcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatccgccaccatgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcg

SEQ ID NO: 116 - DNA, 3825 bp, Artificial Sequence
  source: /note=“Description of Artificial Sequence: Synthetic polynucleotide”
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 117 - protein, 1273 aa, Artificial Sequence
  source: /note=“Description of Artificial Sequence: Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 118 - DNA, 10086 bp, Artificial Sequence
  source: /note=“Description of Artificial Sequence: Synthetic polynucleotide”
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttcgcctggagacgccatccacgctgttttgacctccatagaagacaccgcgatcggatccacaagtttgtacaaaaaagcaggctgccaccatgttcgtgtttctggtgctgctgccactggtgtccagtcagtgcgtgaacttcaccaaccgaacacagctgccatccgcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaactaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgtccgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaccatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagtacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccatcaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgatagtaaacccagctttcttgtacaaagtggtctcgagttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 119 - DNA, 3822 bp, Artificial Sequence
  source: /note=“Description of Artificial Sequence: Synthetic polynucleotide”
atgttcgtgtttctggtgctgctgccactggtgtccagtcagtgcgtgaacttcaccaaccgaacacagctgccatccgcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaactaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgtccgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaccatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagtacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccatcaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 120 - protein, 1273 aa, Artificial Sequence
  source: /note=“Description of Artificial Sequence: Synthetic polypeptide”
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 121 - protein, 9 aa, Severe acute respiratory syndrome coronavirus 2
LTDEMIAQY

SEQ ID NO: 122 - protein, 9 aa, Severe acute respiratory syndrome coronavirus 2
FLHVTYVPA

SEQ ID NO: 123 - protein, 9 aa, Severe acute respiratory syndrome coronavirus 2
KIYSKHTPI

SEQ ID NO: 124 - protein, 10 aa, Severe acute respiratory syndrome coronavirus 2
KLPDDFTGCV

SEQ ID NO: 125 - protein, 9 aa, Severe acute respiratory syndrome coronavirus 2
LLFNKVTLA

SEQ ID NO: 126 - protein, 9 aa, Severe acute respiratory syndrome coronavirus 2
RLDKVEAEV

SEQ ID NO: 127 - protein, 10 aa, Severe acute respiratory syndrome coronavirus 2
RLITGRLQSL

SEQ ID NO: 128 - protein, 9 aa, Severe acute respiratory syndrome coronavirus 2
RLQSLQTYV

SEQ ID NO: 129 - protein, 9 aa, Severe acute respiratory syndrome coronavirus 2
TLDSKTQSL

SEQ ID NO: 130 - protein, 9 aa, Severe acute respiratory syndrome coronavirus 2
VLNDILSRL

SEQ ID NO: 131 - protein, 9 aa, Severe acute respiratory syndrome coronavirus 2
YLQPRTFLL

SEQ ID NO: 132 - protein, 9 aa, Severe acute respiratory syndrome coronavirus 2
RLNEVAKNL

SEQ ID NO: 133 - protein, 9 aa, Severe acute respiratory syndrome coronavirus 2
VVFLHVTYV

SEQ ID NO: 134 - protein, 9 aa, Severe acute respiratory syndrome coronavirus 2
NLNESLIDL

SEQ ID NO: 135 - protein, 9 aa, Severe acute respiratory syndrome coronavirus 2
FIAGLIAIV

SEQ ID NO: 136 - protein, 9 aa, Severe acute respiratory syndrome coronavirus 2
KCYGVSPTK

SEQ ID NO: 137 - protein, 9 aa, Severe acute respiratory syndrome coronavirus 2
GVYFASTEK

SEQ ID NO: 138 - protein, 9 aa, Severe acute respiratory syndrome coronavirus 2
RLFRKSNLK

SEQ ID NO: 139 - protein, 9 aa, Severe acute respiratory syndrome coronavirus 2
GTHWFVTQR

SEQ ID NO: 140 - protein, 10 aa, Severe acute respiratory syndrome coronavirus 2
KWPWYIWLGF

SEQ ID NO: 141 - protein, 9 aa, Severe acute respiratory syndrome coronavirus 2
QYIKWPWYI

SEQ ID NO: 142 - protein, 9 aa, Severe acute respiratory syndrome coronavirus 2
NYNYLYRLF

SEQ ID NO: 143 - protein, 9 aa, Severe acute respiratory syndrome coronavirus 2
RFDNPVLPF

SEQ ID NO: 144 - protein, 9 aa, Severe acute respiratory syndrome coronavirus 2
SPRRARSVA

SEQ ID NO: 145 - protein, 8 aa, Severe acute respiratory syndrome coronavirus 2
APHGVVFL

SEQ ID NO: 146 - protein, 9 aa, Severe acute respiratory syndrome coronavirus 2
QPTESIVRF

SEQ ID NO: 147 - protein, 9 aa, Severe acute respiratory syndrome coronavirus 2
LPFNDGVYF

SEQ ID NO: 148 - protein, 9 aa, Severe acute respiratory syndrome coronavirus 2
IPFAMQMAY

SEQ ID NO: 149 - protein, 9 aa, Severe acute respiratory syndrome coronavirus 2
YEQYIKWPW

SEQ ID NO: 150 - protein, 15 aa, Severe acute respiratory syndrome coronavirus 2
ITRFQTLLALHRSYL

SEQ ID NO: 151 - protein, 15 aa, Severe acute respiratory syndrome coronavirus 2
FNGLTVLPPLLTDEM

SEQ ID NO: 152 - protein, 18 aa, Severe acute respiratory syndrome coronavirus 2
QLIRAAEIRASANLAATK